(19) United States
(12) Patent Application Publication
(10) Pub. No.: US 2002/0197265 A1
(43) Pub. Date: Dec. 26, 2002

(54) Methods and compounds for the treatment of immunologically - mediated diseases of the respiratory system using mycobacterium vaccae

(76) Inventors: James D. Watson, Auckland (NZ); Paul L.J. Tan, Auckland (NZ)

Correspondence Address: Janet Sleath, SPECKMAN LAW GROUP, Suite 100, 1501 Western Avenue, Seattle, WA, 98101, US

(21) Appl. No.: 10/051,643

(22) Filed: Jan. 18, 2002

Related U.S. Application Data
(63) Continuation of application No. 09/156,181, filed on Sep. 17, 1998.
Application No. 09/156,181 is a continuation-in-part of application No. 08/996,624, filed on Dec. 23, 1997, now abandoned.

(51) Int. Cl. A61K 39/00; A61K 39/38

(52) U.S. Cl. 424/184.1

(57) ABSTRACT

Methods for the prevention and treatment by immunotherapy of lung immune disorders, including infection with mycobacteria such as M. tuberculosis or M. avium, sarcoidosis, asthma, allergic rhinitis and lung cancers are provided, such methods comprising administering a composition having antigenic and/or adjuvant properties. Compositions which may be usefully employed in the inventive methods include inactivated M. vaccae cells, delipidated and deglycolipidated M. vaccae cells, M. vaccae culture filtrate and compounds present in or derived therefrom, together with combinations of such components.

### REFERENCE TO RELATED APPLICATIONS

This application is a continuation of U.S. patent application Ser. No. 09/156,181, filed Sep. 17, 1998, which is a continuation-in-part of U.S. patent application Ser. No. 08/996,624, filed Dec. 23, 1997.

### TECHNICAL FIELD

The present invention relates generally to methods for treatment of diseases of the respiratory system which result from immune disorders. In particular, the invention is related to the use of compositions comprising inactivated Mycobacterium vaccae (M. vaccae), and/or compounds prepared from M. vaccae for the treatment and prevention of respiratory and/or lung disorders including mycobacterial infections, such as Mycobacterium tuberculosis and Mycobacterium avium, and for the treatment of disorders, such as sarcoidosis, asthma and lung cancers.

### BACKGROUND OF THE INVENTION

Tuberculosis is a chronic, infectious disease, that is caused by infection with Mycobacterium tuberculosis (M. tuberculosis). It is a major disease in developing countries, as well as an increasing problem in developed areas of the world, with about 8 million new cases and 3 million deaths each year. Although the infection may be asymptomatic for a considerable period of time, the disease is most commonly manifested as a chronic inflammation of the lungs, resulting in fever and respiratory symptoms. If left untreated, significant morbidity and death may result.

Although tuberculosis can generally be controlled using extended antibiotic therapy, such treatment is not sufficient to prevent the spread of the disease. Infected individuals may be asymptomatic, but contagious, for some time. In addition, although compliance with the treatment regimen is critical, patient behavior is difficult to monitor. Some patients do not complete the course of treatment, which can lead to ineffective treatment and the development of drug resistant mycobacteria.

Inhibiting the spread of tuberculosis requires effective vaccination and accurate, early diagnosis of the disease. Currently, vaccination by subcutaneous or intradermal injection with live bacteria is the most efficient method for inducing protective immunity. The most common mycobacterium employed for this purpose is Bacillus Calmette-Guerin (BCG), an avirulent strain of Mycobacterium bovis (M. bovis). However, the safety and efficacy of BCG is a source of controversy and some countries, such as the United States, do not vaccinate the general public. Diagnosis of M. tuberculosis infection is commonly achieved using a skin test, which involves intradermal exposure to tuberculin PPD (protein-purified derivative). Antigen-specific T cell responses result in measurable induration at the injection site by 48-72 hours after injection, thereby indicating exposure to mycobacterial antigens. Sensitivity and specificity have, however, been a problem with this test, and individuals vaccinated with BCG cannot be distinguished from infected individuals.

A less well-known mycobacterium that has been used for immunotherapy for tuberculosis and also leprosy, by subcutaneous or intradermal injection, is Mycobacterium vaccae (M. vaccae), which is non-pathogenic in humans. However, there is less information on the efficacy of M. vaccae compared with BCG, and it has not been used widely to vaccinate the general public. M. bovis BCG and M. vaccae are believed to contain antigenic compounds that are recognized by the immune system of individuals exposed to infection with M. tuberculosis.

Several patents and other publications disclose treatment of various conditions by administering mycobacteria, including M. vaccae, or certain mycobacterial fractions. U.S. Pat. No. 4,716,038 discloses diagnosis of, vaccination against and treatment of autoimmune diseases of various types, including arthritic diseases, by administering mycobacteria, including M. vaccae. U.S. Pat. No. 4,724,144 discloses an immunotherapeutic agent comprising antigenic material derived from M. vaccae for treatment of mycobacterial diseases, especially tuberculosis and leprosy, and as an adjuvant to chemotherapy. International Patent Publication WO 91/01751 discloses the use of antigenic and/or immunoregulatory material from M. vaccae as an immunoprophylactic to delay and/or prevent the onset of AIDS. International Patent Publication WO 94/06466 discloses the use of antigenic and/or immunoregulatory material derived from M. vaccae for therapy of HIV infection, with or without AIDS and with or without associated tuberculosis.

U.S. Pat. No. 5,599,545 discloses the use of mycobacteria, especially whole, inactivated M. vaccae, as an adjuvant for administration with antigens which are not endogenous to M. vaccae. This publication theorises that the beneficial effect as an adjuvant may be due to heat shock protein 65 (hsp 65). International Patent Publication WO 92/08484 discloses the use of antigenic and/or immunoregulatory material derived from M. vaccae for the treatment of uveitis. International Patent Publication WO 93/16727 discloses the use of antigenic and/or immunoregulatory material derived from M. vaccae for the treatment of mental diseases associated with an autoimmune reaction initiated by an infection. International Patent Publication WO 95/26742 discloses the use of antigenic and/or immunoregulatory material derived from M. vaccae for delaying or preventing the growth or spread of tumours. International Patent Publication WO 91/02542 discloses the use of autoclaved M. vaccae in the treatment of chronic inflammatory disorders in which a patient demonstrates an abnormally high release of IL-6 and/or TNF or in which the patient's IgG shows an abnormally high proportion of agalactosyl IgG. Among the disorders mentioned in this publication are psoriasis, rheumatoid arthritis, mycobacterial disease, Crohn's disease, primary biliary cirrhosis, sarcoidosis, ulcerative colitis, systemic lupus erythematosus, multiple sclerosis, Guillain-Barre syndrome, primary diabetes mellitus, and some aspects of graft rejection.

M. vaccae is apparently unique among known mycobacterial species in that heat-killed preparations retain vaccine and immunotherapeutic properties. For example, M. tuberculosis BCG vaccines, used for vaccination against tuberculosis, employ live strains. Heat-killed M. bovis BCG and M. tuberculosis have no protective properties when employed in vaccines. A number of compounds have been isolated from a range of mycobacterial species which have adjuvant properties. The effect of such adjuvants is essentially to stimulate a particular immune response mechanism against an antigen from another species.

There are two general classes of compounds which have been isolated from mycobacterial species that exhibit adjuvant properties. The first are water soluble wax D fractions (R. G. White, I. Bernstock, R. G. S. Johns and E. Lederer, Immunology, 1:54, 1958; U.S. Pat. No. 4,036,953). The second are muramyl dipeptide-based substances (N-acetyl glucosamine and N-glycolymuramic acid in approximately equimolar amounts) as described in U.S. Pat. Nos. 3,956,481 and 4,036,953. These compounds differ from the delipidated and deglycolipidated M. vaccae (DD-M. vaccae) of the present invention in the following aspects of their composition:

1. They are water-soluble agents, whereas DD-M. vaccae is insoluble in aqueous solutions.

2. They consist of a range of small oligomers of the mycobacterial cell wall unit, either extracted from bacteria by various solvents, or digested from the cell wall by an enzyme. In contrast, DD-M. vaccae contains highly polymerised cell wall.

3. All protein has been removed from their preparations by digestion with proteolytic enzymes. The only constituents of their preparations are the components of the cell wall peptidoglycan structure, namely alanine, glutamic acid, diaminopimelic acid, N-acetyl glucosamine, and N-glycolylmuramic acid. In contrast, DD-M. vaccae contains 50% w/w protein, comprising a number of distinct protein species.

The delivery of vaccines by nasal aerosols to reach lung tissue, or by oral delivery to the gastrointestinal tract has been generally limited to attenuated strains of virus. For example, vaccination against poliovirus has employed oral delivery of attenuated strains of this virus since the development of the Sabin vaccine. Aviron Incorporated and the National Institute of Allergy and Infectious Diseases in the United States have recently reported the successful use of an influenza vaccine administered in a nasal spray. In this case, a live attenuated influenza strain provided 93% protection against influenza in young children. Vaccines consisting of killed viruses or bacteria, or of recombinant proteins have not been delivered by nasal aerosol or oral delivery. There are several reasons for this. There are few reports of successful immunization resulting in T cell immunity or antibody synthesis employing these agents administered nasally. Further, oral delivery of proteins and killed organisms often results in the development of tolerance, which is exactly the reverse outcome sought in successful immunization.

Sarcoidosis is a disease of unknown cause characterized by granulomatous inflammation affecting many organs of the body and especially the lungs, lymph nodes and liver. Sarcoid granulomata are composed of mononuclear phagocytes, with epithelioid and giant cells in their center, and T lymphocytes. CD4 T lymphocytes are closely associated with the epithelioid cells while both CD4 and CD8 T lymphocytes accumulate at the periphery. The characteristic immunological abnormalities in sarcoidosis include peripheral blood and bronchoalveolar lavage hyper-globulinaemia and depression of ‘delayed type’ hypersensitivity reactions in the skin to tuberculin and other similar antigens, such as Candida and mumps. Peripheral blood lymphocyte numbers are reduced and CD4: CD8 ratios in peripheral blood are depressed to approximately 1-1.5:1. These are not manifestations of a generalized immune defect, but rather the consequence of heightened immunological activity which is ‘compartmentalized’ to sites of disease activity. In patients with pulmonary sarcoidosis, the total number of cells recovered by bronchoalveolar lavage is increased five- to ten-fold and the proportion of lymphocytes increased from the normal of less than 10-14% to between 15% and 50%. More than 90% of the lymphocytes recovered are T lymphocytes and the CD4:CD8 ratio has been reported to be increased from the value of 1.8:1 in normal controls to 10.5:1. The T lymphocytes are predominantly of the Th1 class, producing IFN-γ and IL-2 cytokines, rather than of the Th2 class. Following treatment, the increase in Th1 lymphocytes in sarcoid lungs is corrected.

Sarcoidosis involves the lungs in nearly all cases. Even when lesions are predominantly seen in other organs, subclinical lung involvement is usually present. While some cases of sarcoidosis resolve spontaneously, approximately 50% of patients have at least a mild degree of permanent organ dysfunction. In severe cases, lung fibrosis develops and progresses to pulmonary failure requiring lung transplantation. The mainstay of treatment for sarcoidosis is corticosteroids. Patients initially responding to corticosteroids often relapse and require treatment with other immunosuppressive drugs such as methotrexate or cyclosporine.

Asthma is a common disease, with a high prevalence in the developed world. Asthma is characterized by increased responsiveness of the tracheobronchial tree to a variety of stimuli, the primary physiological disturbance being reversible airflow limitation, which may be spontaneous or drug-related, and the pathological hallmark being inflammation of the airways. Clinically, asthma can be subdivided into extrinsic and intrinsic variants.

Extrinsic asthma has an identifiable precipitant, and can be thought of as being atopic, occupational and drug-induced. Atopic asthma is associated with the enhancement of a Th2-type of immune response with the production of specific immunoglobulin E (IgE), positive skin tests to common aeroallergens and/or atopic symptoms. It can be divided further into seasonal and perennial forms according to the seasonal timing of symptoms. The airflow obstruction in extrinsic asthma is due to nonspecific bronchial hyperesponsiveness caused by inflammation of the airways. This inflammation is mediated by chemicals released by a variety of inflammatory cells including mast cells, eosinophils and lymphocytes. The actions of these mediators result in vascular permeability, mucus secretion and bronchial smooth muscle constriction. In atopic asthma, the immune response producing airway inflammation is brought about by the Th2 class of T cells which secrete IL-4, IL-5 and IL-10. It has been shown that lymphocytes from the lungs of atopic asthmatics produce IL-4 and IL-5 when activated. Both IL-4 and IL-5 are cytokines of the Th2 class and are required for the production of IgE and involvement of eosinophils in asthma. Occupational asthma may be related to the development of IgE to a protein hapten, such as acid anhydrides in plastic workers and plicatic acid in some western red cedar-induced asthma, or to non-IgE related mechanisms, such as that seen in toluene diisocyanate-induced asthma. Drug-induced asthma can be seen after the administration of aspirin or other non-steroidal anti-inflammatory drugs, most often in a certain subset of patients who may display other features such as nasal polyposis and sinusitis. Intrinsic or cryptogenic asthma is reported to develop after upper respiratory tract infections, but can arise de novo in middle-aged or older people, in whom it is more difficult to treat than extrinsic asthma.

Asthma is ideally prevented by the avoidance of triggering allergens but this is not always possible nor are triggering allergens always easily identified. The medical therapy of asthma is based on the use of corticosteroids and bronchodilator drugs to reduce inflammation and reverse airway obstruction. In chronic asthma, treatment with corticosteroids leads to unacceptable adverse side effects.

Another disorder with a similar immune abnormality to asthma is allergic rhinitis. Allergic rhinitis is a common disorder and is estimated to affect at least 10% of the population. Allergic rhinitis may be seasonal (hay fever) caused by allergy to pollen. Non-seasonal or perennial rhinitis is caused by allergy to antigens such as those from house dust mite or animal dander.

The abnormal immune response in allergic rhinitis is characterised by the excess production of IgE antibodies specific against the allergen. The inflammatory response occurs in the nasal mucosa rather than further down the airways as in asthma. Like asthma, local eosinophilia in the affected tissues is a major feature of allergic rhinitis. As a result of this inflammation, patients develop sneezing, nasal discharge and congestion. In more severe cases, the inflammation extends to the eyes (conjunctivitis), palate and the external ear. While it is not life threatening, allergic rhinitis may be very disabling, prevent normal activities, and interfere with a person's ability to work. Current treatment involves the use of antihistamines, nasal decongestants and, as for asthma, sodium cromoglycate and corticosteroids.

Lung cancer is the leading cause of death from cancer. The incidence of lung cancer continues to rise and the World Health Organization estimates that by 2000 AD there will be 2 million new cases annually. Lung cancers may be broadly classified into two categories: small cell lung cancer (SCLC) which represents 20-25% of all lung cancers, and non-small cell lung cancer (NSCLC) which accounts for the remaining 75%. The majority of SCLC is caused by tobacco smoke. SCLC tend to spread early and 90% of patients present at diagnosis with involvement of the mediastinal lymph nodes in the chest. SCLC is treated by chemotherapy, or a combination of chemotherapy and radiotherapy. Complete response rates vary from 10% to 50%. For the rare patient without lymph node involvement, surgery followed by chemotherapy may result in cure rates exceeding 60%. The prognosis for NSCLC is more dismal, as most patients have advanced disease by the time of diagnosis. Surgical removal of the tumor is possible in a very small number of patients and the five year survival rate for NSCLC is only 5-10%.

The factors leading to the development of lung cancer are complex and multiple. Environmental and genetic factors interact and cause sequential and incremental abnormalities which lead to uncontrolled cell proliferation, invasion of adjacent tissues and spread to distant sites.

Both cell-mediated and humoral immunity have been shown to be impaired in patients with lung cancer. Radiotherapy and chemotherapy further impair the immune function of patients. Attempts have been made to immunize patients with inactivated tumour cells or tumour antigens to enhance host anti-tumor response. Bacillus Calmette-Guerin (BCG) has been administered into the chest cavity following lung cancer surgery to augment non-specific immunity. Attempts have been made to enhance anti-tumor immunity by giving patients lymphocytes treated ex vivo with interleukin-2. These lymphokine-activated lymphocytes acquire the ability to kill tumor cells. The current immunotherapies for lung cancer are still at a developmental stage and their efficacies yet to be established for the standard management of lung cancer.

There thus remains a need in the art for effective compositions and methods for the prevention and treatment of immune disorders of the respiratory system.

### SUMMARY OF THE INVENTION

A Briefly stated, the present invention provides methods for the prevention and treatment by immunotherapy of immune disorders of the respiratory system, including infection with mycobacteria such as M. tuberculosis or M. avium, sarcoidosis, asthma, allergic rhinitis and lung cancers. The inventive methods comprise administering a composition having antigenic and/or adjuvant properties. In one aspect of the present invention, the compositions are administered to the airways leading to or located within the lungs, preferably by inhalation through the nose or mouth, and are preferably administered in aerosol forms. The compositions may also be administered by intradermal or subcutaneous routes.

In one embodiment, compositions which may be usefully employed in the inventive methods comprise a component selected from the group consisting of inactivated M. vaccae cells, M. vaccae culture filtrate, delipidated and deglycolipidated M. vaccae cells, and combinations thereof.

In a first aspect, the inventive methods comprise administering one or more doses of a composition including a component selected from the group consisting of inactivated M. vaccae cells, delipidated and deglycolipidated M. vaccae cells, and components that are present in or derived from either M. vaccae cells or M. vaccae culture filtrate. Specific examples of components present in or derived from either M. vaccae cells or M. vaccae culture filtrate include isolated polypeptides that comprise a sequence selected from the group consisting of SEQ ID NO: 1-4, 9-16, 18-21, 23, 25, 26, 28, 29, 44, 45, 47, 52-55, 63, 64, 70, 75, 89, 94, 98, 100-105, 109, 110, 112, 121, 124, 125, 134, 135, 140, 141, 143, 145, 147, 152, 154, 156, 158, 160, 165, 166, 170, 172, 174, 177, 178, 181, 182, 184, 186, 187, 192, 194, 201, 203, 205 and 207, and variants thereof.

In a second aspect, the inventive methods comprise administering a first dose of a composition at a first point in time and administering a second dose of the composition at a second, subsequent, point in time. Preferably, the multiple doses are administered at intervals of about 2-4 weeks. In one embodiment, compositions which may be usefully employed in such methods comprise a component selected from the group consisting of inactivated M. vaccae cells, M. vaccae culture filtrate, delipidated and deglycolipidated M. vaccae cells, and constituents and combinations thereof.

Additional compositions which may be usefully employed in the inventive methods comprise a DNA molecule encoding one or more of the above polypeptides. Compositions comprising a fusion protein, wherein the fusion protein includes at least one of the above polypeptides, together with DNA molecules encoding such fusion proteins, may also be usefully employed in the methods of the present invention.

The compositions employed in the present invention may additionally include a non-specific immune response enhancer, or adjuvant. Such adjuvants may include M. vaccae culture filtrate, delipidated and deglycolipidated M. vaccae cells, or an isolated polypeptide comprising a sequence provided in SEQ ID NO: 89, 117, 160, 162 or 201, or a variant thereof.

In another aspect, the present invention provides methods for the treatment of a disorder of the respiratory system in a patient by the administration of one or more of the above compositions, wherein the disorder is characterized by the presence of eosinophilia in the tissues of the respiratory system. Examples of such diseases include asthma and allergic rhinitis. In a related aspect, the present invention provides methods for the reduction of eosinophilia, in a patient, such methods comprising administering at least one of the compositions disclosed herein. Typically, the reduction in eosinophilia will vary between about 20% and about 80%. The percentage of reduction in lung eosinophilia can be determined by measuring the number of eosinophils in bronchoalveolar lavage fluid before and after treatment as described below in Example 2.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 compares the stimulation of IL-12 production in macrophages by different concentrations of heat-killed M. vaccae, lyophilized M. vaccae, delipidated and deglycolipidated M. vaccae and M. vaccae glycolipids.

FIG. 2 compares the stimulation of interferon-gamma production in spleen cells from SCID mice by different concentrations of heat-killed M. vaccae, lyophilized M. vaccae, delipidated and deglycolipidated M. vaccae and M. vaccae glycolipids.

FIGS. 3A(i)-(iv) illustrate the non-specific immune amplifying effects of 10 μg, 100 μg and 1 mg autoclaved M. vaccae and 75 μg unfractionated culture filtrates of M. vaccae, respectively. FIGS. 3B(i) and (ii) illustrate the non-specific immune amplifying effects of autoclaved M. vaccae, and delipidated and deglycolipidated M. vaccae, respectively. FIG. 3C(i) illustrates the non-specific immune amplifying effects of whole autoclaved M. vaccae. FIG. 3C(ii) illustrates the non-specific immune amplifying effects of soluble M. vaccae proteins extracted with SDS from delipidated and deglycolipidated M vaccae. FIG. 3C(iii) illustrates that the adjuvant effect of the preparation of FIG. 3C(ii) is destroyed by treatment with the proteolytic enzyme pronase. FIG. 3D illustrates the non-specific immune amplifying effects of heat-killed M. vaccae (FIG. 3D(i)), whereas heat-killed preparations of M. tuberculosis (FIG. 3D(ii)), M. bovis BCG (FIG. 3D(iii)), M. phlei (FIG. 3D(iv)) and M. smegmatis (FIG. 3D(v)) did not demonstrate non-specific immune amplifying effects.

FIGS. 4A and B show the percentage of eosinophils in mice immunized intranasally with either 10 or 1000 μg of heat-killed M. vaccae or 200-100 μg of DD-M. vaccae, respectively, 4 weeks prior to challenge with ovalbumin, as compared to control mice. FIGS. 4C and D show the percentage of eosinophils in mice immunized intranasally with either 100 μg of heat-killed M. vaccae or 200 μg of DD-M. vaccae, respectively, as late as one week prior to challenge with ovalbumin. FIG. 4E shows the percentage of eosinophils in mice immunized either intranasally (i.n.) or subcutaneously (s.c.) with either BCG of the Pasteur strain (BCG-P), BCG of the Connought strain (BCG-C), 1 mg of heat-killed M. vaccae, or 200 μg of DD-M. vaccae prior to challenge with ovalbumin.

FIG. 5 illustrates the non-specific immune amplifying property of each of the recombinant proteins GV27, 27A, 27B, 23 and 45 in the generation of cytotoxic T cells to a structurally unrelated protein, ovalbumin.

### DETAILED DESCRIPTION OF THE INVENTION

As detailed below, the inventors have successfully induced T cell immune responses and protective immunity against M. tuberculosis in rodents and non-human primates following immunization with heat-killed M. vaccae and various M. vaccae derivatives through the lung. The inventors have additionally demonstrated that both heat-killed M. vaccae and M. vaccae derivatives are able to inhibit the development of an allergic immune response in the lungs when administered either intranasally or subcutaneously in a rodent model of asthma.

Effective vaccines that provide protection against infectious microorganisms contain at least two functionally different components. The first is a polypeptide antigen, which is processed by macrophages and other antigen-presenting cells and displayed for CD4⁺ T cells or for CD8⁺ T cells. This antigenic component forms the “specific” target of an immune response. The second component of a vaccine is a non-specific immune response amplifier, termed an adjuvant, with which the antigen is mixed or is incorporated into. An adjuvant amplifies immune responses to a structurally unrelated compound or antigen. Several known adjuvants are prepared from microbes such as Bordetella pertussis, M. tuberculosis and M. bovis BCG. Adjuvants may also contain components designed to protect polypeptide antigens from degradation, such as aluminum hydroxide or mineral oil. While the antigenic component of a vaccine contains polypeptides that direct the immune attack against a specific pathogen, such as M. tuberculosis, the adjuvant is often capable of broad use in many different vaccine formulations. Some known proteins, such as bacterial enterotoxins, can function both as an antigen to elicit a specific immune response and as an immune response amplifier to enhance immune responses to other antigens.

Certain pathogens, such as M. tuberculosis, as well as certain cancers, are effectively contained by an immune attack directed by CD4⁺ T cells, known as cell-mediated immunity. Other pathogens, such as poliovirus, also require antibodies, produced by B cells, for containment. These different classes of immune attack (T cell or B cell) are controlled by different subpopulations of CD4⁺ T cells, commonly referred to as Th1 and Th2 cells.

The two types of Th cell subsets have been well characterized in a murine model and are defined by the cytokines they release upon activation. The Th1 subset secretes IL-2, IFN-γ and tumor necrosis factor, and mediates macrophage activation and delayed-type hypersensitivity response. The Th2 subset releases IL-4, IL-5, IL-6 and IL-10, which stimulate B cell activation. The Th1 and Th2 subsets are mutually inhibiting, so that IL-4 inhibits Th1-type responses, and IFN-γ inhibits Th2-type responses. Similar Th1 and Th2 subsets have been found in humans, with release of the identical cytokines observed in the murine model. Amplification of Th1-type immune responses is central to a reversal of disease state in many disorders, including disorders of the respiratory system such as tuberculosis, sarcoidosis, asthma, allergic rhinitis and lung cancers.

Inactivated M. vaccae and compounds derived from M. vaccae have both antigenic and adjuvant properties. The methods of the present invention employ compounds from M. vaccae and/or its culture filtrates that have T cell enhancing immune activities. Mixtures of such compounds are particularly useful in redirecting immune activities of T cells in patients. While it is well known that all mycobacteria contain many cross-reacting antigens, it is not known whether they contain adjuvant compounds in common. As shown below, inactivated M. vaccae cells and a modified (delipidated and deglycolipidated) form of M. vaccae have been found to have adjuvant properties which are not shared by a number of other mycobacterial species. Furthermore, it has been found that M. vaccae produces compounds in its own culture filtrate which amplify a Th1-type immune response to M. vaccae antigens also found in culture filtrate, as well as to antigens from other sources.

The present invention provides methods for the immunotherapy of respiratory and/or lung disorders, including tuberculosis, sarcoidosis, asthma, allergic rhinitis and lung cancers, in a patient to enhance Th1-type immune responses. In one embodiment, the compositions are delivered directly to the mucosal surfaces of airways leading to and/or within the lungs. However, the compositions may also be administered via intradermal or subcutaneous routes. Compositions which may be usefully employed in the inventive methods comprise at least one of the following components: inactivated M. vaccae cells; M. vaccae culture filtrate; delipidated and deglycolipidated M. vaccae cells (DD-M. vaccae); and compounds present in or derived from M. vaccae and/or its culture filtrate. As illustrated below, administration of such compositions, results in specific T cell immune responses and enhanced protection against M. tuberculosis infection. Administration of such compositions is also effective in the treatment of asthma. While the precise mode of action of these compositions in the treatment of diseases such as asthma is unknown, they are believed to suppress an asthma-inducing Th2 immune response.

Inactivated M. vaccae are M. vaccae that have either been killed by means of heat, as detailed below, or subjected to radiation, such as ⁶⁰cobalt at a dose of 2.5 megarads. As detailed in Example 3, the inventors have shown that removal of the glycolipid constituents from M. vaccae results in the removal of molecular components that stimulate interferon-gamma production in natural killer (NK) cells, thereby significantly reducing the non-specific production of a cytokine that has numerous harmful side-effects.

As used herein the term “respiratory system” refers to the lungs, nasal passageways, trachea and bronchial passageways.

As used herein the term “airways leading to or located in the lung” includes the nasal passageways, mouth, tonsil tissue, trachea and bronchial passageways.

As used herein, a “patient” refers to any warm-blooded animal, preferably a human. Such a patient may be afflicted with disease or may be free of detectable disease. In other words, the inventive methods may be employed to induce protective immunity for the prevention or treatment of disease.

As used herein the term “inactivated M. vaccae” refers to M. vaccae cells that have either been killed by means of heat, as detailed below in Examples 1 and 2, or subjected to radiation, such as ⁶⁰Cobalt at a dose of 2.5 megarads. As used herein, the term “modified M. vaccae” includes delipidated M. vaccae cells, deglycolipidated M. vaccae cells and M. vaccae cells that have been both delipidated and deglycolipidated.

Delipidated and deglycolipidated M. vaccae may be prepared as described below in Example 1. As detailed below, the inventors have shown that removal of the glycolipid constituents from M. vaccae results in the removal of molecular components that stimulate interferon-gamma production in natural killer (NK) cells, thereby significantly reducing the non-specific production of a cytokine that has numerous harmful side-effects.

Compounds present in or derived from M. vaccae cells and/or from M. vaccae culture filtrate that may be usefully employed in the inventive methods include M. vaccae polypeptides, or variants thereof. Such polypeptides possess antigenic and/or adjuvant properties. In specific embodiments, such polypeptides comprise a sequence selected from the group consisting of SEQ ID NO: 1-4, 9-16, 18-21, 23, 25, 26, 28, 29, 44, 45, 47, 52-55, 63, 64, 70, 75, 89, 94, 98, 100-105, 109, 110, 112, 121, 124, 125, 134, 135, 140, 141, 143, 145, 147, 152, 154, 156, 158, 160, 165, 166, 170, 172, 174, 177, 178, 181, 182, 184, 186, 187, 192, 194, 201, 203, 205 and 207.

As used herein, the term “polypeptide” encompasses amino acid chains of any length, including full length proteins (i.e. antigens), wherein the amino acid residues are linked by covalent peptide bonds. A polypeptide may comprise an immunogenic portion of an antigen. Such polypeptides may consist entirely of the immunogenic portion, or may contain additional sequences. The additional sequences may be derived from the native M. vaccae antigen or may be heterologous, and such sequences may (but need not) be immunogenic. As detailed below, polypeptides of the present invention may be isolated from M. vaccae cells or culture filtrate, or may be prepared by synthetic or recombinant means.

“Immunogenic”, as used herein, refers to the ability of a polypeptide to elicit an immune response in a patient, such as a human, or in a biological sample. In particular, immunogenic antigens are capable of stimulating cell proliferation, interleukin-12 production or interferon-γ production in biological samples comprising one or more cells selected from the group of T cells, NK cells, B cells and macrophages, where the cells are derived from an individual previously exposed to tuberculosis. Exposure to an immunogenic antigen usually results in the generation of immune memory such that upon re-exposure to that antigen, an enhanced and more rapid response occurs.

Immunogenic portions of the antigens described herein may be prepared and identified using well known techniques, such as those summarised in Paul, Fundamental Immunology, 3d ed., Raven Press, 1993, pp. 243-247. Such techniques include screening polypeptide portions of the native antigen or protein for immunogenic properties. The representative proliferation and cytokine production assays described herein may be employed in these screens. An immunogenic portion of an antigen is a portion that, within such representative assays, generates an immune response (e.g., cell proliferation, interferon-γ production or interleukin-12 production) that is substantially similar to that generated by the full-length antigen. In other words, an immunogenic portion of an antigen may generate at least about 20%, preferably about 65%, and most preferably about 100% of the proliferation induced by the full-length antigen in the model proliferation assay described herein. An immunogenic portion may also, or alternatively, stimulate the production of at least about 20%, preferably about 65% and most preferably about 100%, of the interferon-γ and/or interleukin-12 induced by the full length antigen in the model assay described herein.

A M. vaccae adjuvant is a compound found in M. vaccae cells or M. vaccae culture filtrates which non-specifically stimulates immune responses. Adjuvants enhance the immune response to immunogenic antigens and the process of memory formation. In the case of M. vaccae proteins, these memory responses favor Th1-type immunity. Adjuvants are also capable of stimulating interleukin-12 production or interferon-γ production in biological samples comprising one or more cells selected from the group of T cells, NK cells, B cells and macrophages, where the cells are derived from healthy individuals. Adjuvants may or may not stimulate cell proliferation. Such M. vaccae adjuvants include, for example, polypeptides comprising a sequence recited in SEQ ID NO: 89, 117, 160, 162 or 201.

The compositions for use in the inventive methods also encompass variants of the above polypeptides. Such variants include, but are not limited to, naturally occurring allelic variants. As used herein, the term “variant” covers any sequence which exhibits at least about 50%, more preferably at least about 70% and more preferably yet, at least about 90% overall identity to a sequence of the present invention. In one embodiment, a “variant” is any sequence which has at least about a 99% probability of being the same as the inventive sequence. The probability and/or identity for DNA sequences is measured using the computer algorithm BLASTN and that for protein sequences is measured using the computer algorithm BLASTP (Altschul, S. F. et al. Nucleic Acids Res. 25:3389-3402, 1997). The term “variants” thus encompasses sequences wherein the probability of finding a match by chance (smallest sum probability), is less than about 1% as measured by any of the above tests. Both BLASTN and BLASTP are available on the NCBI anonymous FTP server under/blast/executables/. For BLASTP the following running parameters are preferred: blastall -p blastp -d swissprotdb -e 10 -G 1 -E 1 -v 50 -b 50 -i

queryseq -o results

-p Program Name [String]

-d Database [String]

-e Expectation value (E) [Real]

-G Cost to open a gap (zero invokes default behavior) [Integer]

-E Cost to extend a gap (zero invokes default behavior) [Integer]

-v Number of one-line descriptions (v) [Integer]

-b Number of alignments to show (b) [Integer]

-I Query File [File In]

-o BLAST report Output File [File Out] Optional For BLASTN the following running parameters are preferred: blastall -p blastn d embldb -e 10 -G 1 -E 1 -r 2 -v 50 -b 50 -i queryseq -o results

-p Program Name [String]

-d Database [String]

-e Expectation value (E) [Real]

-G Cost to open a gap (zero invokes default behavior) [Integer]

-E Cost to extend a gap (zero invokes default behavior) [Integer]

-r Reward for a nucleotide match (blastn only) [Integer]

-v Number of one-line descriptions (v) [Integer]

-b Number of alignments to show (b) [Integer]

-I Query File [File In]

-o BLAST report Output File [File Out] Optional

Variant nucleotide sequences will generally hybridize to the recited nucleotide sequence under stringent conditions. As used herein, “stringent conditions” refers to prewashing in a solution of 6× SSC, 0.2% SDS; hybridizing at 65° C., 6× SSC, 0.2% SDS overnight; followed by two washes of 30 minutes each in 1× SSC, 0.1% SDS at 65° C. and two washes of 30 minutes each in 0.2× SSC, 0.1% SDS at 65° C.

Portions and other variants of M. vaccae polypeptides may be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J Am. Chem. Soc. 85:2149-2146, 1963. Equipment for automated synthesis of polypeptides is commercially available from suppliers such as Perkin Elmer/Applied BioSystems, Inc. (Foster City, Calif.), and may be operated according to the manufacturer's instructions. Variants of a native antigen or adjuvant may be prepared using standard mutagenesis techniques, such as oligonucleotide-directed site specific mutagenesis. Sections of the DNA sequence may also be removed using standard techniques to permit preparation of truncated polypeptides.

A polypeptide of the present invention may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

In general, M. vaccae polypeptides, and DNA sequences encoding such polypeptides, may be prepared using any of a variety of procedures. For example, soluble polypeptides may be isolated from M. vaccae culture filtrate as described below. Polypeptides may also be produced recombinantly by inserting a DNA sequence that encodes the polypeptide into an expression vector and expressing the polypeptides in an appropriate host. Any of a variety of expression vectors known to those of ordinary skill in the art may be employed. Expression may be achieved in any appropriate host cell that has been transformed or transfected with an expression vector containing a DNA molecule that encodes recombinant polypeptide. Suitable host cells include prokaryotes, yeast and higher eukaryotic cells. Preferably, the host cells employed are E. coli, yeast or a mammalian cell line such as COS or CHO. The DNA sequences expressed in this manner may encode naturally occurring polypeptides, portions of naturally occurring polypeptides, or other variants thereof.

DNA sequences encoding M. vaccae polypeptides may be obtained by screening an appropriate M. vaccae cDNA or genomic DNA library for DNA sequences that hybridize to degenerate oligonucleotides derived from partial amino acid sequences of isolated soluble polypeptides. Suitable degenerate oligonucleotides may be designed and synthesized, and the screen may be performed as described, for example, in Maniatis et al., Molecular Cloning—A Laboratory Manual, Cold Spring Harbor Laboratories, Cold Spring Harbor, N.Y., 1989. As described below, polymerase chain reaction (PCR) may be employed to isolate a nucleic acid probe from genomic DNA, or a cDNA or genomic DNA library. The library screen may then be performed using the isolated probe.

DNA molecules encoding M. vaccae polypeptides may also be isolated by screening an appropriate M. vaccae cDNA or genomic DNA expression library with anti-sera (e.g., rabbit or monkey) raised specifically against M. vaccae polypeptides, as detailed below.

Regardless of the method of preparation, the polypeptides described herein have the ability to induce and/or enhance an immunogenic response. More specifically, the polypeptides have the ability to induce and/or enhance cell proliferation and/or cytokine production (for example, interferon-γ and/or interleukin-12 production) in T cells, NK cells, B cells or macrophages derived from an M. tuberculosis-immune individual. A M. tuberculosis-immune individual is one who is considered to be resistant to the development of tuberculosis by virtue of having mounted an effective T cell response to M. tuberculosis. Such individuals may be identified based on a strongly positive (i.e., greater than about 10 mm diameter induration) intradermal skin test response to tuberculosis proteins (PPD), and an absence of any symptoms of tuberculosis infection.

Assays for cell proliferation or cytokine production in T cells, NK cells, B cell macrophages may be performed, for example, using the procedures described below. The selection of cell type for use in evaluating an immune response to an antigen will depend on the desired response. For example, interleukin-12 or interferon-γ production is most readily evaluated using preparations containing T cells, NK cells, B cells and macrophages derived from individuals using methods well known in the art. For example, a preparation of peripheral blood mononuclear cells (PBMCs) may be employed without further separation of component cells.

PBMCs may be prepared, for example, using density centrifugation through FiColl™ (Winthrop Laboratories, NY). T cells for use in the assays described herein may be purified directly from PBMCs.

In general, regardless of the method of preparation, the polypeptides employed in the inventive methods are prepared in an isolated, substantially pure, form. Preferably, the polypeptides are at least about 80% pure, more preferably at least about 90% pure and most preferably at least about 99% pure. In certain preferred embodiments, described in detail below, the isolated polypeptides are incorporated into pharmaceutical compositions or vaccines for use in one or more of the methods disclosed herein.

Fusion proteins comprising a first and a second inventive polypeptide disclosed herein or, alternatively, a polypeptide disclosed herein and a known M. tuberculosis antigen, such as the 38 kDa antigen described in Andersen and Hansen, Infect. Immun. 57:2481-2488, 1989, together with variants of such fusion proteins, may also be employed in the inventive methods. Such fusion proteins may include a linker peptide between the first and second polypeptides. A DNA sequence encoding such a fusion protein is constructed using known recombinant DNA techniques to assemble separate DNA sequences encoding the first and second polypeptides into an appropriate expression vector. The end of a DNA sequence encoding the first polypeptide is ligated, with or without a peptide linker, to the 5′ end of a DNA sequence encoding the second polypeptide so that the reading frames of the sequences are in phase to permit mRNA translation of the two DNA sequences into a single fusion protein that retains the biological activity of both the first and the second polypeptides.

A peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad Sci. USA 83:8258-8262, 1986; U.S. Pat. No. 4,935,233 and U.S. Pat. No. 4,751,180. The linker sequence may be from 1 to about 50 amino acids in length. Peptide linker sequences are not required when the first and second polypeptides have non-essential N-terminal amino acid regions that can be used to separate the functional domains and prevent steric interference. The ligated DNA sequences encoding the fusion proteins are cloned into suitable expression systems using techniques known to those of ordinary skill in the art.

For use in the inventive methods, the inactivated M. vaccae cell, M. vaccae culture filtrate, delipidated and deglycolipidated M. vaccae cells, or compounds present in or derived from M. vaccae and/or its culture filtrate are generally present within a pharmaceutical composition or a vaccine. Pharmaceutical compositions may comprise one or more polypeptides, each of which may contain one or more of the above sequences (or variants thereof), and a physiologically acceptable carrier. Vaccines may comprise one or more components selected from the group consisting of inactivated M. vaccae cells, M. vaccae culture filtrate, delipidated and deglycolipidated M. vaccae cells, and compounds present in or derived from M. vaccae and/or its culture filtrate, together with a non-specific immune response amplifier. Such pharmaceutical compositions and vaccines may also contain other mycobacterial polypeptides, either, as discussed above, incorporated into a fusion protein or present within a separate polypeptide.

Alternatively, a vaccine or pharmaceutical composition for use in the methods of the present invention may contain DNA encoding one or more polypeptides as described above, such that the polypeptide is generated in situ. In such vaccines, the DNA may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminator signal). Bacterial delivery systems involve the administration of a bacterium (such as Bacillus-Calmette-Geurin) that expresses an immunogenic portion of the polypeptide on its cell surface. In a preferred embodiment, the DNA may be introduced using a viral expression system (e.g., vaccinia or other pox virus, retrovirus, or adenovirus), which may involve the use of a non-pathogenic, or defective, replication competent virus. Techniques for incorporating DNA into such expression systems are well known in the art. The DNA may also be “naked,” as described, for example, in Ulmer et al., Science 2.59:1745-1749, 1993 and reviewed by Cohen, Science 259:1691-1692. 1993. The uptake of naked DNA may be increased by coating the DNA onto biodegradable beads, which are efficiently transported into the cells.

In one embodiment, the pharmaceutical composition or vaccine is in a form suitable for delivery to the mucosal surfaces of the airways leading to or within the lungs. For example, the pharmaceutical composition or vaccine may be suspended in a liquid formulation for delivery to a patient in an aerosol form or by means of a nebulizer device similar to those currently employed in the treatment of asthma. In other embodiments, the pharmaceutical composition or vaccine is in a form suitable for administration by injection (intracutaneous, intramuscular, intravenous or subcutaneous) or orally. While any suitable carrier known to those of ordinary skill in the art may be employed in the pharmaceutical compositions of this invention, the type of carrier will depend on the suitability for the chosen route of administration. Examples of carriers which may be usefully employed in the inventive methods include mannitol, lactose, starch, magnesium stearate, sodium saccharine, cellulose, glucose, sucrose, and magnesium carbonate. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. Any of a variety of adjuvants may be employed in the vaccines of this invention to non-specifically enhance the immune response.

The preferred frequency of administration and effective dosage will vary from individual to individual. In general, the amount of polypeptide present in a dose (or produced in situ by the DNA in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 mL to about 5 mL. In the case of inactivated M. vaccae cells, the amount present in a dose preferably ranges from about 10 to about 1000 mg, and is more preferably about 500 mg. For DD-M. vaccae, the amount present in a dose preferably ranges from about 10 μg to about 1000 μg, more preferably from about 50 μg to about 200 μg. For both inactivated M. vaccae and DD-M. vaccae, the number of doses may range from 1 to about 10 administered over a period of up to 12 months.

The word “about,” when used in this application with reference to the amount of active component in a dose, contemplates a variance of up to 5% from the stated amount. The word “about,” when used with reference to a percentage reduction of eosinophils, contemplates a variance of up to 10% from the stated percentage.

The following examples are offered by way of illustration and are not limiting.

### EXAMPLE 1

### Effect of Intradermal and Intra-lung Routes of Immunization with M. vaccae on Tuberculosis in Cynomolgous Monkeys

This example illustrates the effect of immunization with heat-killed M. vaccae or M. vaccae culture filtrate through intradermal and intralung routes in cynomolgous monkeys prior to challenge with live M. tuberculosis.

M. vaccae (ATCC Number 15483) was cultured in sterile Medium 90 (yeast extract, 2.5 g/l; tryptone, 5g/l; glucose, 1 g/l) at 37° C. The cells were harvested by centrifugation, and transferred into sterile Middlebrook 7H9 medium (Difco Laboratories, Detroit, Mich., USA) with glucose at 37° C. for one day. The medium was then centrifuged to pellet the bacteria, and the culture filtrate removed. The bacterial pellet was resuspended in phosphate buffered saline at a concentration of 10 mg/ml, equivalent to 10¹⁰ M. vaccae organisms per ml. The cell suspension was then autoclaved for 15 min at 120° C. The culture filtrate was passaged through a 0.45 μM filter into sterile bottles.

Five groups of cynomolgous monkeys were used, with each group containing 2 monkeys. Two groups of monkeys were immunized with whole heat-killed M. vaccae either intradermally or intralung; two groups of monkeys were immunized with M. vaccae culture filtrate either intradermally or intralung; and a control group received no immunizations. All immunogens were dissolved in phosphate buffered saline. The composition employed for immunization, amount of immunogen, and route of administration for each group of monkeys are provided in Table 1. Prior to immunization, all monkeys were weighed (Wt kg), body temperature was measured (temp), and a blood sample taken for determination of erythrocyte sedimentation rate (ESR mm/hr) and lymphocyte proliferation (LPA) to an in vitro challenge with purified protein (PPD) prepared from Mycobacterium bovis. Both ESR and LPA have been used as indicators of inflammatory T cell responses. At day 33 post-immunization these measurements were repeated. At day 34, all monkeys received a second immunization using the same amount of M. vaccae and route of immunization as the initial immunization. On day 62, body weight, temperature, ESR and LPA to PPD were measured, then all monkeys were infected with 10³ colony forming units of the Erdman strain of Mycobacterium tuberculosis by inserting the organisms directly in the right lungs of immunized animals. Twenty eight days following infection, body weight, temperature, ESR and LPA to PPD were measured in all monkeys, and the lungs were x-rayed to determine whether infection with live M. tuberculosis had resulted in the onset of pneumonia.

TABLE 1
COMPARISON OF INTRADERMAL AND
INTRALUNG ROUTES OF IMMUNIZATION

| | | | |
|---|---|---|---|
| | Identification | | |
| Group | Number of | Amount of | Route of |
| Number | Monkey | Immunogen | Immunization |

| | | | |
|---|---|---|---|
| 1 | S3101-E | 0 | — |
| (Controls) | 3144-B | 0 | — |
| 2 | 4080-B | 500 μg | intradermal |
| (Immunized | 3586-B | 500 μg | intradermal |
| with heat-killed | | | |
| M. vaccae) | | | |
| 3 | 3534-C | 500 μg | intralung |
| (Immunized | 3160-A | 500 μg | intralung |
| with heat-killed | | | |
| M. vaccae) | | | |
| 4 | 3564-B | 100 μg | intradermal |
| (Immunized | 3815-B | 100 μg | intradermal |
| with culture filtrate) | | | |
| 5 | 4425-A | 100 μg | intralung |
| (Immunized | 2779-D | 100 μg | intralung |
| with culture filtrate) | | | |

The results of these studies are provided below in Tables 2A-E and are summarized below:

Table 2A—Twenty-eight days after infection with M. tuberculosis Erdman, chest x-rays of control (non-immunized) monkeys revealed haziness over the right suprahilar regions of both animals, indicating the onset of pneumonia. This progressed and by day 56 post-infection x-rays indicated disease in both lungs. As expected, as disease progressed both control animals lost weight and showed significant LPA responses to PPD, indicating strong T cell reactivity to M. tuberculosis. The ESR measurements were variable but consistent with strong immune reactivity.

Table 2B—The two monkeys immunized twice with 500 μg M. vaccae intradermally showed no sign of lung disease 84 days post-infection with M. tuberculosis. The LPA responses to PPD indicated there was immune reactivity to M. tuberculosis, and both animals continued to gain weight, a consistent indication of a lack of disease.

Table 2C—The two monkeys immunized twice with 500 μg M. vaccae intralung showed almost identical results to those animals of Table 2B. There was no sign of lung disease 84 days post infection with M. tuberculosis, with consistent weight gains. Both animals showed LPA response to PPD in the immunization phase (day 0-62) and post-infection, indicating strong T cell reactivity had developed as a result of using the lung as the route of immunization and subsequent infection.

Immunization twice with 500 μg of whole M. vaccae has consistently shown protective effects agsint subsequent infection with live M. tuberculosis. The data presented in Tables 2D and 2E show the effects of immunization with 100 μg of M. vaccae culture filtrate. Monkeys immunized intradermally showed signs of developing disease 84 days post-infection, while in those immunized intralung, one animal showed disease after 56 days and one animal showed disease 84 days post-infection. This was a significant delay in disease onset indicating that the immunization process had resulted in some protective immunity.

TABLE 2A
CONTROL MONKEYS

| | | Wt. | | ESR | LPA | LPA | X-Ray |
|---|---|---|---|---|---|---|---|
| ID# | Days | Kgs | Temp. | Mm/hr | PPD10 | PPD1 | Remarks |
| S3101E | 0 | 2.17 | 37.0 | 0 | 0.47 | 1.1 | Negative |
| | 34 | 1.88 | 37.3 | ND | 0.85 | 1.4 | ND |
| | 62 | 2.02 | 36.0 | ND | 1.3 | 1.5 | ND |

→ Time of Infection

| | | | | | | |
|---|---|---|---|---|---|---|
| 28 | 2.09 | 38.0 | 2 | 1.3 | 3.7 | Positive |
| 56 | 1.92 | 37.2 | 20 | 5.6 | 9.1 | Positive |
| 84 | 1.81 | 37.5 | 8 | 4.7 | 5.6 | Positive |

| | 121 | DIED | | | | | |
|---|---|---|---|---|---|---|---|
| | | | | | LPA | LPA | |
| | | Wt. | | ESR | PPD | PPD | X-Ray |
| ID# | Days | Kgs | Temp. | Mm/hr | 10 μg | 1 μg | Remarks |
| 3144-B | 0 | 2.05 | 36.7 | 0 | 0.87 | 1.8 | Negative |
| | 34 | 1.86 | 37.6 | ND | 2.2 | 1.4 | ND |
| | 62 | 1.87 | 36.5 | ND | 1.6 | 1.6 | ND |

→ Time of Infection

| | | | | | | |
|---|---|---|---|---|---|---|
| 28 | 2.10 | 38.0 | 0 | 12 | 8.7 | Positive |
| 56 | 1.96 | 37.6 | 0 | 29.6 | 21.1 | Positive |
| 84 | 1.82 | 37.3 | 4 | 45.3 | 23.4 | Positive |

| | |
|---|---|
| 131 | DIED |

ND = Not Done

TABLE 2B
MONKEYS IMMUNIZED WITH WHOLE HEAT-KILLED
M. VACCAE (500 μg) INTRADERMAL

| | | | | | LPA | LPA | |
|---|---|---|---|---|---|---|---|
| | | Wt. | | ESR | PPD | PPD | X-Ray |
| ID# | Days | Kgs | Temp. | mm/hr | 10 μg | 1 μg | Remarks |
| 4080-B | 0 | 2.05 | 37.1 | 1 | 1.1 | 0.77 | Negative |
| | 34 | 1.97 | 38.0 | ND | 1.7 | 1.4 | ND |
| | 62 | 2.09 | 36.7 | ND | 1.5 | 1.5 | ND |

→ Time of Infection

| | 28 | 2.15 | 37.6 | 0 | 2.6 | 2.1 | Negative |
|---|---|---|---|---|---|---|---|
| | 56 | 2.17 | 37.6 | 0 | 8.2 | 7.6 | Negative |
| | 84 | 2.25 | 37.3 | 0 | 3.8 | 2.8 | Negative |
| | 178 | DIED | | | | | |
| | | | | | LPA | LPA | |
| | | Wt. | | ESR | PPD | PPD | X-Ray |
| ID# | Days | Kgs | Temp. | mm/hr | 10 μg | 1 μg | Remarks |
| 3586-B | 0 | 2.29 | 37.0 | 0 | 1.1 | 1.4 | Negative |
| | 34 | 2.22 | 38.0 | ND | 1.9 | 1.6 | ND |
| | 62 | 2.39 | 36.0 | ND | 1.3 | 1.6 | ND |

→ Time of Infection

| | | | | | | |
|---|---|---|---|---|---|---|
| 28 | 2.31 | 38.2 | 0 | 3.2 | 2.6 | Negative |
| 56 | 2.32 | 37.2 | 0 | 7.8 | 4.2 | Negative |
| 84 | 2.81 | 37.4 | 0 | 3.4 | 1.8 | Negative |

| | |
|---|---|
| 197 | DIED |

ND = Not Done

TABLE 2C
MONKEYS IMMUNIZED WITH WHOLE HEAT-KILLED
M. VACCAE (500 μg) INTRALUNG

| | | | | | LPA | LPA | |
|---|---|---|---|---|---|---|---|
| | | Wt. | | ESR | PPD | PPD | X-Ray |
| ID# | Days | Kgs | Temp. | mm/hr | 10 μg | 1 μg | Remarks |
| 3534-C | 0 | 2.15 | 36.8 | 0 | 1.7 | 1.3 | Negative |
| | 34 | 2.00 | 37.8 | ND | 4.4 | 1.4 | ND |
| | 62 | 2.13 | 36.4 | ND | 3.2 | 1.9 | ND |

→ Time of Infection

| | 28 | 2.38 | 37.7 | 0 | 1.9 | 2.6 | Negative |
|---|---|---|---|---|---|---|---|
| | 56 | 2.42 | 37.8 | 0 | 5.3 | 4.7 | Negative |
| | 84 | 2.46 | 37.1 | 1 | 3.1 | 3.2 | Negative |
| | 210 | No sign of lung disease | | | | | Negative |
| 3160-A | 0 | 2.17 | 37.3 | 0 | 1.2 | 0.79 | Negative |
| | 34 | 1.98 | 37.1 | ND | 3.9 | 7.8 | ND |
| | 62 | 2.17 | 36.9 | ND | 1.7 | 2.4 | ND |

→ Time of Infection

| | | | | | | |
|---|---|---|---|---|---|---|
| 28 | 2.38 | 37.7 | 0 | 1.9 | 2.6 | Negative |
| 56 | 2.42 | 37.8 | 0 | 5.3 | 4.7 | Negative |
| 84 | 2.46 | 37.1 | 1 | 3.1 | 3.2 | Negative |

| | | |
|---|---|---|
| 210 | Stable lung disease | Positive |

ND = Not Done

TABLE 2D
MONKEYS IMMUNIZED WITH CULTURE FILTRATE
(100 μg) INTRADERMAL

| | | | | | LPA | LPA | |
|---|---|---|---|---|---|---|---|
| | | Wt. | | ESR | PPD | PPD | X-Ray |
| ID# | Days | Kgs | Temp. | mm/hr | 10 μg | 1 μg | Remarks |
| 3564-B | 0 | 2.40 | 37.2 | 0 | 1.4 | 1.4 | Negative |
| | 34 | 2.42 | 38.1 | ND | 3.3 | 2.7 | ND |
| | 62 | 2.31 | 37.1 | ND | 3.1 | 3.4 | ND |

→ Time of Infection

| | | | | | | | |
|---|---|---|---|---|---|---|---|
| | 28 | 2.41 | 38.6 | 13 | 24 | 13.6 | Negative |
| | 56 | 2.38 | 38.6 | 0 | 12.7 | 12.0 | Negative |
| | 84 | 2.41 | 38.6 | 2 | 21.1 | 11.8 | Positive |
| | 140 | | | | | | Died |
| 3815-B | 0 | 2.31 | 36.3 | 0 | 1.0 | 1.4 | Negative |
| | 34 | 2.36 | 38.2 | ND | 1.9 | 2.0 | ND |
| | 62 | 2.36 | 36.4 | ND | 3.7 | 2.8 | ND |

→ Time of Infection

| | | | | | | |
|---|---|---|---|---|---|---|
| 28 | 2.45 | 37.8 | 0 | 2.1 | 3.3 | Negative |
| 56 | 2.28 | 37.3 | 4 | 8.0 | 5.6 | Negative |
| 84 | 2.32 | 37.4 | 0 | 1.9 | 2.2 | Positive |
| 210 | | | | | | Positive |

ND = Not Done

TABLE 2E
MONKEYS IMMUNIZED WITH CULTURE
FILTRATE (100 μg) INTRALUNG

| | | | | | LPA | LPA | |
|---|---|---|---|---|---|---|---|
| | | Wt. | | ESR | PPD | PPD | X-Ray |
| ID# | Days | Kgs | Temp. | mm/hr | 10 μg | 1 μg | Remarks |
| 4425-A | 0 | 2.05 | 36.0 | 0 | 0.35 | 1.2 | Negative |
| | 34 | 2.0 | 37.6 | ND | 3.0 | 2.4 | ND |
| | 62 | 2.11 | 37.6 | ND | 2.2 | 1.6 | ND |

→ Time of Infection

| | 28 | 2.21 | 38.0 | 0 | 8.4 | 4.1 | Negative |
|---|---|---|---|---|---|---|---|
| | 56 | 2.11 | 37.6 | 0 | 23.9 | 17.7 | Negative |
| | 84 | 2.18 | 37.9 | 0 | 8.4 | 7.2 | Positive |
| | 210 | Stable lung disease | | | | | Positive |
| 2779-D | 0 | 2.56 | 38.6 | 2 | 1.9 | 1.4 | Negative |
| | 28 | 2.55 | 37.9 | ND | 0.78 | 1.1 | ND |
| | 56 | 2.69 | 38.4 | ND | 1.3 | 1.5 | ND |

→ Time of Infection

| | | | | | | |
|---|---|---|---|---|---|---|
| 56 | 2.25 | 39.0 | 24 | ND | ND | Positive |
| 96 | | | | | | Died |

ND = Not Done

### EXAMPLE 2

### Effect of Immunization with M. vaccae on Asthma in Mice

This example illustrates that both heat-killed M. vaccae and DD-M. vaccae, when administered to mice via the intranasal route, are able to inhibit the development of an allergic immune response in the lungs. This was demonstrated in a mouse model of the asthma-like allergen specific lung disease. The severity of this allergic disease is reflected in the large numbers of eosinophils that accumulate in the lungs.

C57BL/6J mice were given 2 μg ovalbumin in 100 μl alum adjuvant by the intraperitoneal route at time 0 and 14 days, and subsequently given 100 μg ovalbumin in 50 μl phosphate buffered saline (PBS) by the intranasal route on day 28. The mice accumulated eosinophils in their lungs as detected by washing the airways of the anaesthetised mice with saline, collecting the washings (broncheolar lavage or BAL), and counting the numbers of eosinophils.

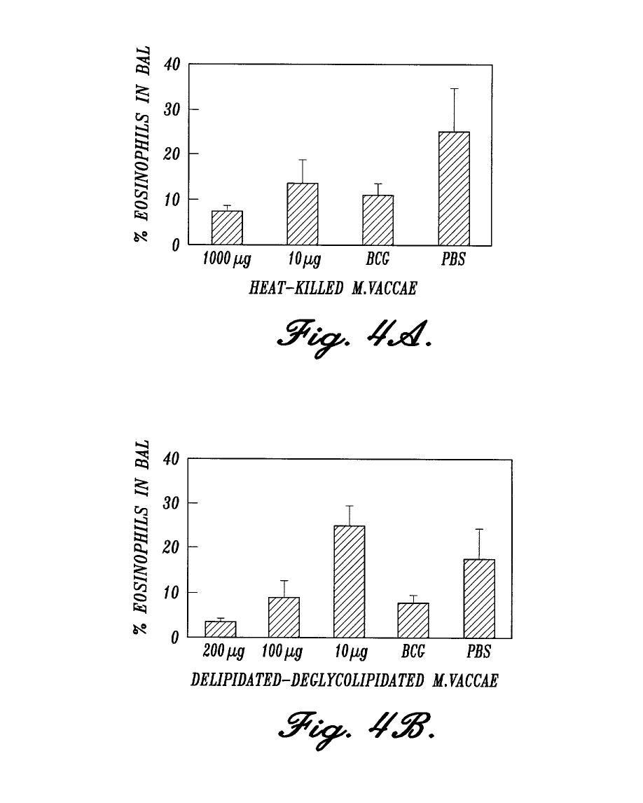

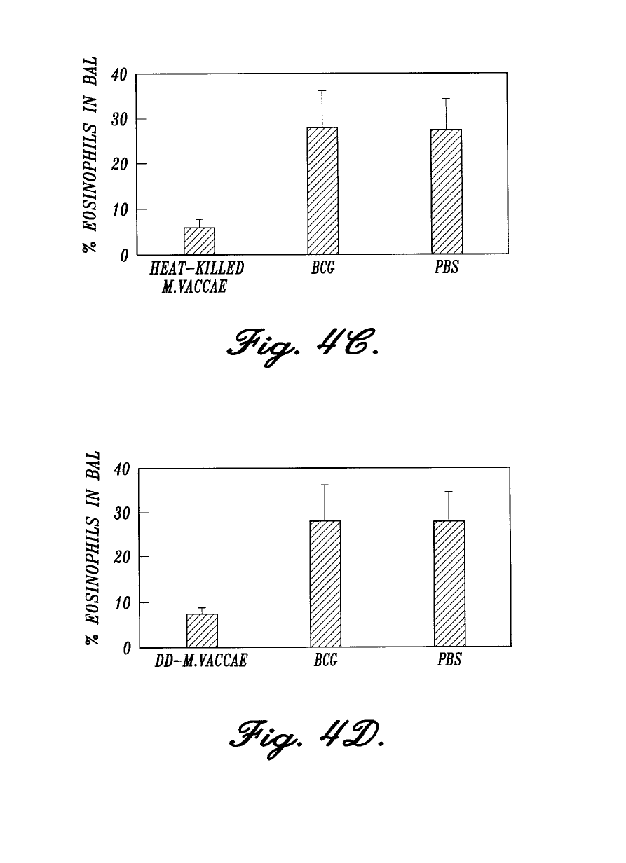

As shown in FIGS. 4A and B, groups of seven mice administered either 10 or 1000 μg of heat-killed M. vaccae (FIG. 4A), or 10, 100 or 200 μg of DD-M. vaccae (FIG. 4B) intranasally 4 weeks before intranasal challenge with ovalbumin, had reduced percentages of eosinophils in the BAL cells collected 5 days after challenge with ovalbumin compared to control mice. Control mice were given intranasal PBS. Live M. bovis BCG at a dose of 2×10⁵ colony forming units also reduced lung eosinophilia. The data in FIGS. 4A and B show the mean and SEM per group of mice.

FIGS. 4C and D show that mice given either 1000 μg of heat-killed M. vaccae (FIG. 4C) or 200 μg of DD-M. vaccae (FIG. 4D) intranasally as late as one week before challenge with ovalbumin had reduced percentages of eosinophils compared to control mice. In contrast, treatment with live BCG one week before challenge with ovalbumin did not inhibit the development of lung eosinophilia when compared with control mice.

As shown in FIG. 4E, immunization with either 1 mg of heat-killed M. vaccae or 200 μg of DD-M. vaccae, given either intranasally (i.n.) or subcutaneously (s.c.), reduced lung eosinophilia following challenge with ovalbumin when compared to control animals given PBS. In the same experiment, immunization with BCG of the Pasteur (BCG-P) and Connought (BCG-C) strains prior to challenge with ovalbumin also reduced the percentage of eosinophils in the BAL of mice.

Eosinophils are blood cells that are prominent in the airways in allergic asthma. The secreted products of eosinophils contribute to the swelling and inflammation of the mucosal linings of the airways in allergic asthma. The data shown in FIGS. 4A-E indicate that treatment with heat-killed M. vaccae or DD-M. vaccae reduces the accumulation of lung eosinophils, and may be useful in reducing inflammation associated with eosinophilia in the airways, nasal mucosal and upper respiratory tract. Administration of heat-killed M. vaccae or DD-M. vaccae may therefore reduce the severity of asthma and diseases that involve similar immune abnormalities, such as allergic rhinitis.

In addition, serum samples were collected from mice in the experiment described in FIG. 4E and antibodies to ovalbumin was measured by standard enzyme-linked immunoassay (EIA). As shown in Table 3 below, sera from mice infected with BCG had higher levels of ovalbumin specific IgG1 than sera from PBS controls. In contrast, mice immunized with M. vaccae or DD-M. vaccae had similar or lower levels of ovalbumin-specific IgG1. As IgG1 antibodies are characteristic of a Th2 immune response, these results are consistent with the suppressive effects of heat-killed M. vaccae and DD-M. vaccae on the asthma-inducing Th2 immune responses.

TABLE 3
LOW ANTIGEN-SPECIFIC IgG1 SERUM LEVELS IN MICE
IMMUNIZED WITH HEAT-KILLED M. VACCAE OR DD-M. VACCAE

Serum IgG1

| Treatment Group | Mean | SEM |
|---|---|---|
| M.vaccae i.n. | 185.00 | 8.3 |
| M. vaccae s.c. | 113.64 | 8.0 |
| DD-M. vaccae i.n. | 96.00 | 8.1 |
| DD-M. vaccae s.c. | 110.00 | 4.1 |
| BCG, Pasteur | 337.00 | 27.2 |
| BCG, Connaught | 248.00 | 46.1 |
| PBS | 177.14 | 11.4 |

Note:
Ovalbumin-specific IgG1 was detected using anti-mouse IgG1 (Serotec). Group means are expressed as the reciprocal of the EU50 end point titre.

### EXAMPLE 3

### Preparation and Immune Modulating Properties of Delipidated and Deglycolipidated (DD-) M. vaccae

This example illustrates the processing of different constituents of M. vaccae and their immune modulating properties.

Heat-killed M. vaccae and M. vaccae culture filtrate

M. vaccae (ATCC Number 15483) was cultured in sterile Medium 90 (yeast extract, 2.5 g/l; tryptone, 5 g/l; glucose 1 g/l) at 37° C. The cells were harvested by centrifugation, and transferred into sterile Middlebrook 7H9 medium (Difco Laboratories, Detroit, Mich., USA) with glucose at 37° C. for one day. The medium was then centrifuged to pellet the bacteria, and the culture filtrate removed. The bacterial pellet was resuspended in phosphate buffered saline at a concentration of 10 mg/ml, equivalent to 10¹⁰ M. vaccae organisms per ml. The cell suspension was then autoclaved for 15 min at 120° C. The culture filtrate was passaged through a 0.45 μM filter into sterile bottles.

Delipidated and Deglycolipidated (DD-) M. vaccae and Compositional Analysis

To prepare delipidated M. vaccae, the autoclaved M. vaccae was pelleted by centrifugation, the pellet washed with water and collected again by centrifugation and then freeze-dried. Freeze-dried M. vaccae was treated with chloroform/methanol (2:1) for 60 mins at room temperature to extract lipids, and the extraction was repeated once. The delipidated residue from chloroform/methanol extraction was further treated with 50% ethanol to remove glycolipids by refluxing for two hours. The 50% ethanol extraction was repeated two times. The pooled 50% ethanol extracts were used as a source of M. vaccae glycolipids (see below). The residue from the 50% ethanol extraction was freeze-dried and weighed. The amount of delipidated and deglycolipidated M. vaccae prepared was equivalent to 11.1% of the starting wet weight of M. vaccae used. For bioassay, the delipidated and deglycolipidated M. vaccae, referred to as DD-M. vaccae, was resuspended in phosphate-buffered saline by sonication, and sterilized by autoclaving.

The compositional analyses of heat-killed M. vaccae and DD-M. vaccae are presented in Table 4. Major changes are seen in the fatty acid composition and amino acid composition of DD-M. vaccae as compared to the insoluble fraction of heat-killed M. vaccae. The data presented in Table 4 shows that the insoluble fraction of heat-killed M. vaccae contains 10% w/w of lipid, and the total amino acid content is 2750 nmoles/mg, or approximately 33% w/w. DD-M. vaccae contains 1.3% w/w of lipid and 4250 nmoles/mg amino acids, which is approximately 51% w/w.

TABLE 4
Compositional analyses of heat-killed M. vaccae and DD-M. vaccae

| | |
|---|---|
| M. vaccae | DD-M. vaccae |

Monosaccharide composition

| | | |
|---|---|---|
| sugar alditol | | |
| Inositol | 3.2% | 1.7% |
| Ribitol* | 1.7% | 0.4% |
| Arabinitol | 22.7% | 27.0% |
| Mannitol | 8.3% | 3.3% |
| Galactitol | 11.5% | 12.6% |
| Glucitol | 52.7% | 55.2% |

Fatty acid composition

| | | |
|---|---|---|
| Fatty acid | | |
| C14:0 | 3.9% | 10.0% |
| C16:0 | 21.1% | 7.3% |
| C16:1 | 14.0% | 3.3% |
| C18:0 | 4.0% | 1.5% |
| C18:1* | 1.2% | 2.7% |
| C18:1w9 | 20.6% | 3.1% |
| C18:1w7 | 12.5% | 5.9% |
| C22:0 | 12.1% | 43.0% |
| C24:1* | 6.5% | 22.9% |

The insoluble fraction of heat-killed M. vaccae contains 10% w/w of lipid, and DD-M. vaccae contains 1.3% w/w of lipid.

Amino Acid Composition

| nmoles/mg | M. vaccae | DD-M. vaccae |
|---|---|---|
| ASP | 231 | 361 |
| THR | 170 | 266 |
| SER | 131 | 199 |
| GLU | 319 | 505 |
| PRO | 216 | 262 |
| GLY | 263 | 404 |
| ALA | 416 | 621 |
| CYS* | 24 | 26 |
| VAL | 172 | 272 |
| MET* | 72 | 94 |
| ILE | 104 | 171 |
| LEU | 209 | 340 |
| TYR | 39 | 75 |
| PHE | 76 | 132 |
| G1cNH2 | 5 | 6 |
| HIS | 44 | 77 |
| LYS | 108 | 167 |
| ARG | 147 | 272 |

The total amino acid content of the insoluble fraction of heat-killed M. vaccae is 2750 nmoles/mg, or approximately 33% w/w. The total amino acid content of DD-M. vaccae is 4250 nmoles/mg, or approximately 51% w/w.

Comparison of composition of DD-M. vaccae with delipidated and deglycolipidated forms of M. tuberculosis and M. smegmatis

Delipidated and deglycolipidated M. tuberculosis and M. smegmatis were prepared using the procedure described above for delipidated and deglycolipidated M. vaccae. As indicated in Table 5, the profiles of the percentage composition of amino acids in DD-M. vaccae, DD-M. tuberculosis and DD-M. smegmatis showed no significant differences. However, the total amount of protein varied—the two batches of DD-M. vaccae contained 34% and 55% protein, whereas DD-M. tuberculosis and DD-M. smegmatis contained 79% and 72% protein, respectively.

TABLE 5
Amino Acid Composition of Delipidated
and Deglycolipidated Mycobacteria

| | DD- | DD- | | |
|---|---|---|---|---|
| Amino | M.vaccae | M.vaccae | DD- | DD- |
| Acid | Batch 1 | Batch 2 | M. smegmatis | M. tuberculosis |
| Asp | 9.5 | 9.5 | 9.3 | 9.1 |
| Thr | 6.0 | 5.9 | 5.0 | 5.3 |
| Ser | 5.3 | 5.3 | 4.2 | 3.3 |
| Glu | 11.1 | 11.2 | 11.1 | 12.5 |
| Pro | 6.1 | 5.9 | 7.5 | 5.2 |
| Gly | 9.9 | 9.7 | 9.4 | 9.8 |
| Ala | 14.6 | 14.7 | 14.6 | 14.2 |
| Cys | 0.5 | 0.5 | 0.3 | 0.5 |
| Val | 6.3 | 6.4 | 7.2 | 7.8 |
| Met | 1.9 | 1.9 | 1.9 | 1.9 |
| Ile | 3.6 | 3.5 | 4.1 | 4.7 |
| Leu | 7.8 | 7.9 | 8.2 | 8.3 |
| Tyr | 1.4 | 1.7 | 1.8 | 1.8 |
| Phe | 4.2 | 4.0 | 3.2 | 3.0 |
| His | 1.9 | 1.8 | 2.0 | 1.9 |
| Lys | 4.1 | 4.0 | 4.1 | 4.2 |
| Arg | 5.8 | 5.9 | 6.2 | 6.4 |
| Total % | 55.1 | 33.8 | 72.1 | 78.5 |
| Protein | | | | |

Analysis of the monosaccharide composition showed significant differences between DD-M. vaccae, and DD-M. tuberculosis and DD-M. smegmatis. The monosaccharide composition of two batches of DD-M. vaccae was the same and differed from that of DD-M. tuberculosis and M. smegmatis. Specifically, DD-M. vaccae was found to contain free glucose while both DD-M. tuberculosis and M. smegmatis contain glycerol, as shown in Table 6.

TABLE 6

| | | |
|---|---|---|
| Alditol | | |
| Acetate | wt % | mol % |

DD-M. vaccae

| | | |
|---|---|---|
| Batch 1 | | |
| Inositol | 0.0 | 0.0 |
| Arabinose | 54.7 | 59.1 |
| Mannose | 1.7 | 1.5 |
| Glucose | 31.1 | 28.1 |
| Galactose | 12.5 | 11.3 |
| | 100.0 | 100.0 |
| Batch 2 | | |
| Inositol | 0.0 | 0.0 |
| Arabinose | 51.0 | 55.5 |
| Mannose | 2.0 | 1.8 |
| Glucose | 34.7 | 31.6 |
| Galactose | 12.2 | 11.1 |
| | 100.0 | 100.0 |

DD-M. smeg

| | | |
|---|---|---|
| Inositol | 0.0 | 0.0 |
| Glycerol | 15.2 | 15.5 |
| Arabinose | 69.3 | 70.7 |
| Xylose | 3.9 | 4.0 |
| Mannose | 2.2 | 1.9 |
| Glucose | 0.0 | 0.0 |
| Galactose | 9.4 | 8.0 |
| | 100.0 | 100.0 |

DD-M. tb

| | | |
|---|---|---|
| Inositol | 0.0 | 0.0 |
| Glycerol | 9.5 | 9.7 |
| Arabinose | 69.3 | 71.4 |
| Mannose | 3.5 | 3.0 |
| Glucose | 1.5 | 1.3 |
| Galactose | 12.4 | 10.7 |
| | 96.2 | 96.0 |

M. vaccae glycolipids

The pooled 50% ethanol extracts described above were dried by rotary evaporation, redissolved in water and freeze-dried. The amount of glycolipid recovered was 1.2% of the starting wet weight of M. vaccae used. For bioassay, the glycolipids were dissolved in phosphate-buffered saline.

Stimulation of Cytokine Synthesis

Whole heat-killed M. vaccae and DD-M. vaccae were shown to have different cytokine stimulation properties. The stimulation of a Th1 immune response is enhanced by the production of interleukin-12 (IL-12) from macrophages. The ability of different M. vaccae preparations to stimulate IL-12 production was demonstrated as follows.

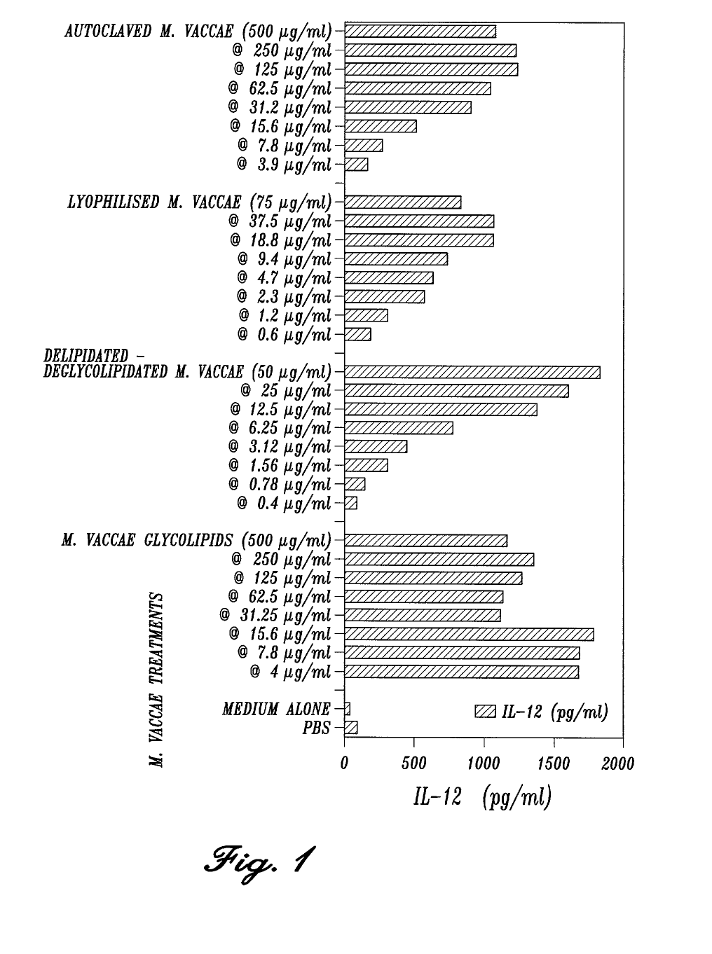

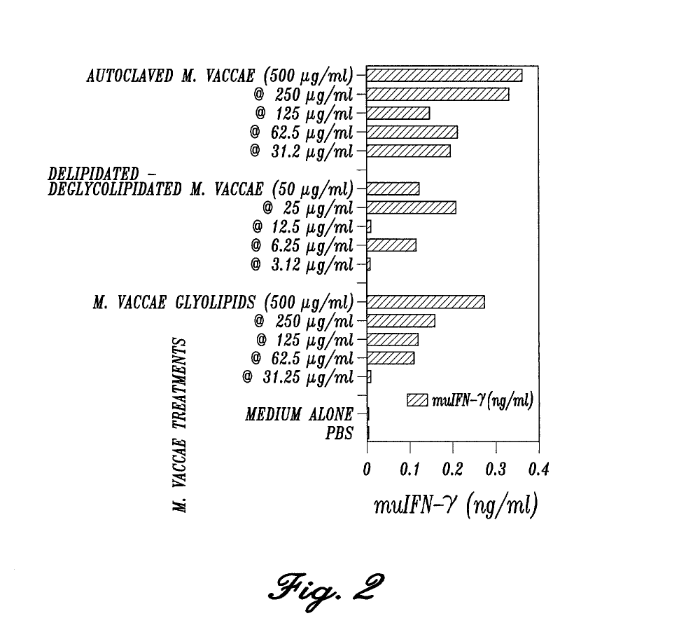

A group of C57BL/6J mice were injected intraperitoneally with DIFCO thioglycolate and, after three days, peritoneal macrophages were collected and placed in cell culture with interferon-gamma for three hours. The culture medium was replaced and various concentrations of whole heat-killed M. vaccae, heat-killed M. vaccae which was lyophilised and reconstituted for use in phospate-buffered saline, DD-M. vaccae, or M. vaccae glycolipids were added. After three days at 37° C., the culture supernatants were assayed for the presence of IL-12 produced by macrophages. As shown in FIG. 1, all the M. vaccae preparations stimulated the production of IL-12 from macrophages.

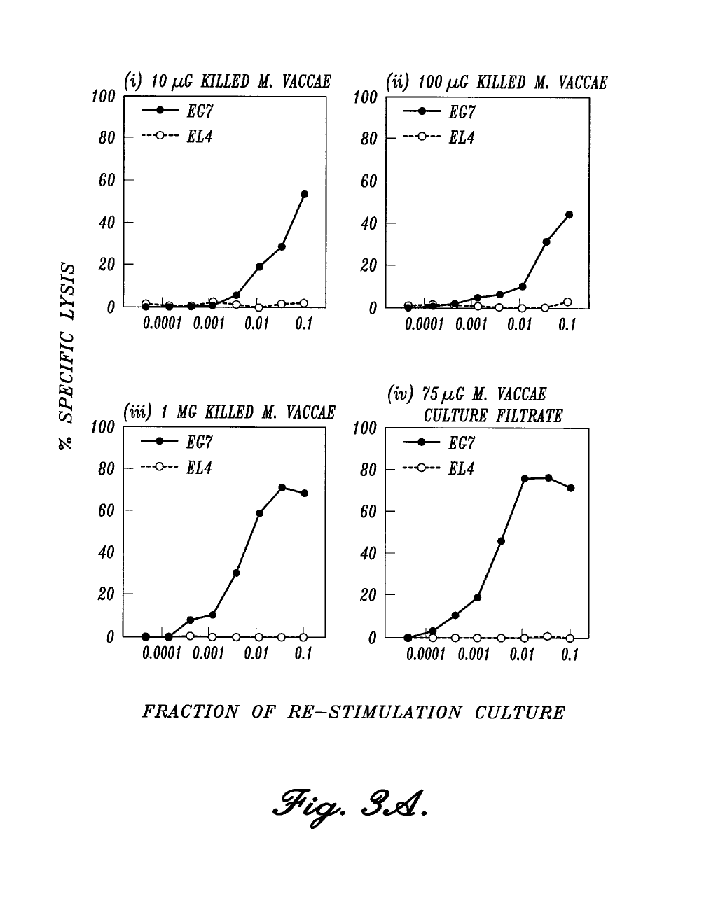

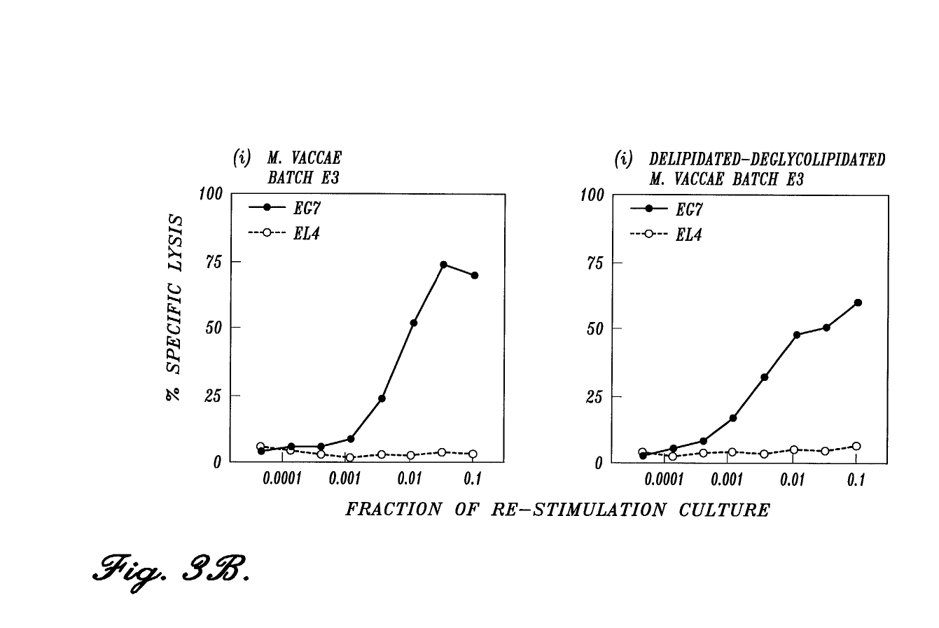

By contrast, these same M. vaccae preparations were examined for the ability to stimulate interferon-gamma production from Natural Killer (NK) cells. Spleen cells were prepared from Severe Combined Immunodeficient (SCID) mice. These populations contain 75-80% NK cells. The spleen cells were incubated at 37° C. in culture with different concentrations of heat-killed M. vaccae, DD-M. vaccae, and M. vaccae glycolipids. The data shown in FIG. 2 demonstrated that, while heat-killed M. vaccae and M. vaccae glycolipids stimulate production of interferon-gamma, DD-M. vaccae stimulated relatively less interferon gamma. The combined data from FIGS. 1 and 2 indicate that compared with M. vaccae, DD-M. vaccae was a better stimulator of IL-12 than interferon gamma.

These findings demonstrate that removal of the lipid glycolipid constituents from M. vaccae results in the removal of molecular components that stimulate interferon-gamma from NK cells, thereby effectively eliminating an important cell source of a cytokine that has numerous harmful side-effects. DD-M. vaccae thus retains Th1 immune enhancing capacity by stimulating IL-12 production, but has lost the non-specific effects that may come through the stimulation of interferon-gamma production from NK cells.

The adjuvant effects of a number of recombinant antigens were determined by measuring stimulation of IL-12 secretion from murine peritoneal macrophages. The cloning and purification of the recombinant proteins are described in Examples 4-10. Recombinant proteins that exhibited adjuvant properties are listed in Table 7.

TABLE 7
Recombinant proteins that exhibit adjuvant properties

Mouse strain

| Antigen | C57BL-6 | Balb/C |
|---|---|---|
| GVs-3 | + | + |
| GVc-4P | + | + |
| GV-5 | + | + |
| GV-5P | + | + |
| GVc-7 | + | + |
| GV-22B | + | ND |
| GV-27 | + | + |
| GV-27A | + | + |
| GV-27B | + | + |
| GV-42 | + | ND |
| DD. M. vaccae | + | + |

ND = not done

Proteins in DD-M. vaccae as non-specific immune amplifiers

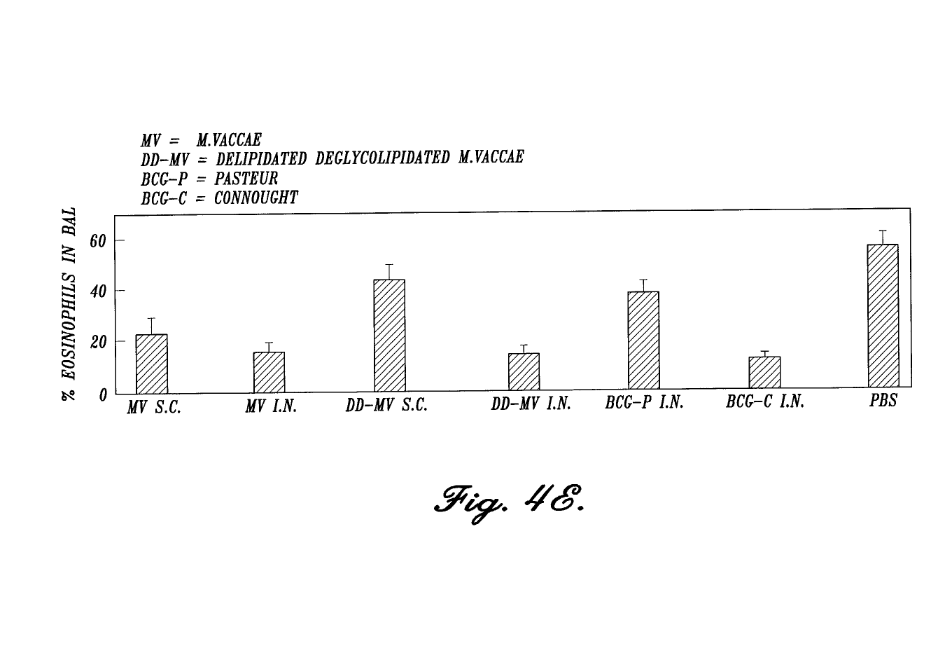

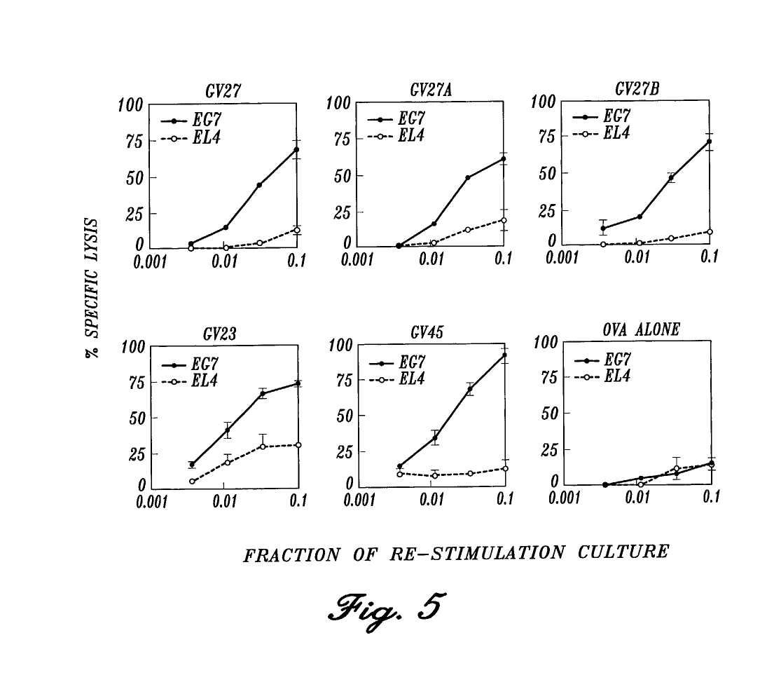

In subsequent experiments, the five proteins GV27, 27A, 27B, 23 and 45 were used as non-specific immune amplifiers with ovalbumin antigen to immunize mice as described below in Example 4. As shown in FIG. 5, 50 μg of any one of the recombinant proteins GV27, 27A, 27B, 23 and 45, when injected with 50-100 μg of ovalbumin, demonstrated adjuvant properties in being able to generate cytotoxic cells to ovalbumin.

### EXAMPLE 4

### The Non-specific Immune Amplifying Properties of Heat-killed M. vaccae, M. vaccae Culture filtrate and DD-M. vaccae

This example illustrates the non-specific immune amplifying or ‘adjuvant’ properties of whole heat-killed M. vaccae, DD-M. vaccae and M. vaccae culture filtrate.

M. vaccae bacteria was cultured, pelleted and autoclaved as described in Example 1. Culture filtrates of live M. vaccae refer to the supernatant from 24 h cultures of M. vaccae in 7H9 medium with glucose. DD-M. vaccae was prepared as described in Example 3.

Killed M. vaccae, DD-M. vaccae and M. vaccae culture filtrate were tested for adjuvant activity in the generation of cytotoxic T cell immune response to ovalbumin, a structurally unrelated protein, in the mouse. This anti-ovalbumin-specific cytotoxic response was detected as follows. Groups of C57BL/6 mice were immunized by the intraperitoneal injection of 100 μg of ovalbumin with the following test adjuvants: heat-killed M. vaccae; DD-M. vaccae; DD-M. vaccae with proteins extracted with SDS; the SDS protein extract treated with Pronase (an enzyme which degrades protein); and either heat-killed M. vaccae, heat-killed M. bovis BCG, M. phlei, M. smegmatis or M. vaccae culture filtrate. After 10 days, spleen cells were stimulated in vitro for a further 6 days with E.G7 cells which are EL4 cells (a C57BL/6-derived T cell lymphoma) transfected with the ovalbumin gene and thus express ovalbumin. The spleen cells were then assayed for their ability to kill non-specifically EL4 target cells or to kill specifically the E.G7 ovalbumin expressing cells. Killing activity was detected by the release of ⁵¹Chromium with which the EL4 and E.G7 cells have been labelled (100 mCi per 2×10⁶), prior to the killing assay. Killing or cytolytic activity is expressed as % specific lysis using the formula:

$$\frac{cpm in test cultures - cpm in control cultures}{total cpm - cpm in control cultures} \times 100 \%$$

It is generally known that ovalbumin-specific cytotoxic cells are generated only in mice immunized with ovalbumin with an adjuvant but not in mice immunized with ovalbumin alone.

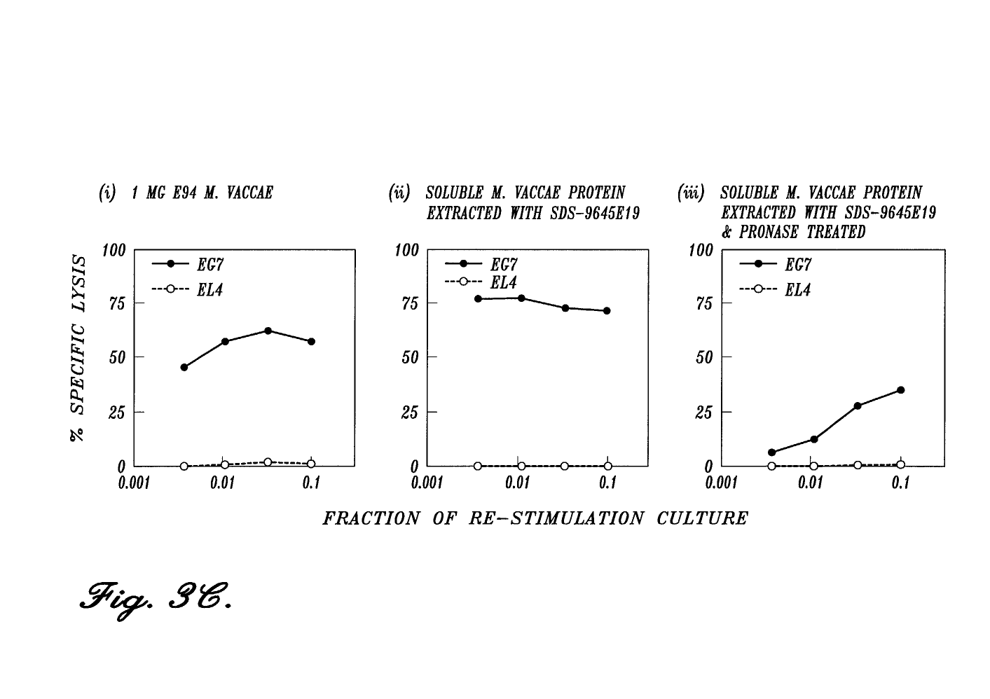

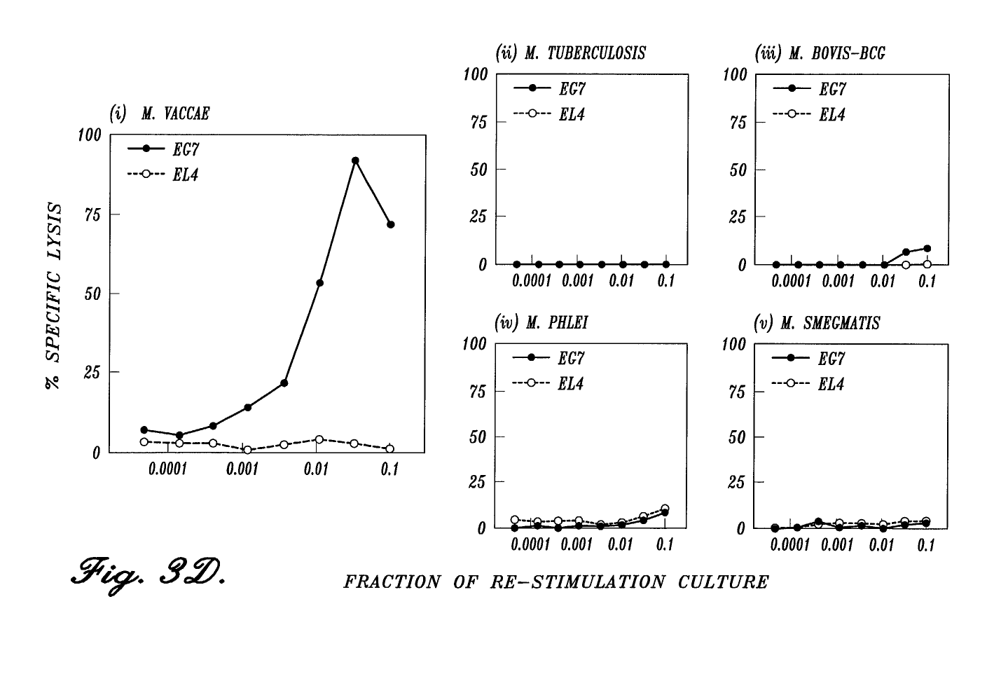

The diagrams that make up FIG. 3 show the effect of various M. vaccae derived adjuvant preparations on the generation of cytotoxic T cells to ovalbumin in C57BL/6 mice. As shown in FIG. 3A, cytotoxic cells were generated in mice immunized with (i) 10 μg, (ii) 100 μg or (iii) 1 mg of autoclaved M. vaccae or (iv) 75 μg of M. vaccae culture filtrate. FIG. 3B shows that cytotoxic cells were generated in mice immunized with (i) 1 mg whole autoclaved M. vaccae or (ii) 100 μg DD-M. vaccae. As shown in FIG. 3C(i), cytotoxic cells were generated in mice immunized with 1 mg heat-killed M. vaccae; FIG. 3C(ii) shows the active material in M. vaccae soluble proteins extracted with SDS from DD-M. vaccae. FIG. 3C(iii) shows that active material in the adjuvant preparation of FIG. 3C(ii) was destroyed by treatment with the proteolytic enzyme Pronase. By way of comparison, 100 μg of the SDS-extracted proteins had significantly stronger immune-enhancing ability (FIG. 3C(ii)) than did 1 mg heat-killed M. vaccae (FIG. 3C(i)). Mice immunized with 1 mg heat-killed M. vaccae (FIG. 3D(i)) generated cytotoxic cells to ovalbumin, but mice immunized separately with 1 mg heat-killed M. tuberculosis (FIG. 3D(ii)), 1 mg M. bovis BCG (FIG. 3D(iii)), 1 mg M. phlei (FIG. 3D(iv)), or 1 mg M. smegmatis (FIG. 3D(v)) failed to generate cytotoxic cells.

The significance of these findings is that heat-killed M. vaccae and DD-M. vaccae have adjuvant properties not seen in other mycobacteria. Further, delipidation and deglycolipidation of M. vaccae removes an NK cell-stimulating activity but does not result in a loss of T cell-stimulating activity.

In subsequent studies, more of the SDS-extracted proteins described above were prepared by preparative SDS-PAGE on a BioRad Prep Cell (Hercules, Calif.). Fractions corresponding to molecular weight ranges were precipitated by trichloroacetic acid to remove SDS before assaying for adjuvant activity in the anti-ovalbumin-specific cytotoxic response assay in C57BL/6 mice as described above. The adjuvant activity was highest in the 60-70 kDa fraction. The most abundant protein in this size range was purified by SDS-PAGE blotted on to a polyvinylidene difluoride (PVDF) membrane and then sequenced. The sequence of the first ten amino acid residues is provided in SEQ ID NO:76. Comparison of this sequence with those in the gene bank as described above, revealed homology to the heat shock protein 65 (GroEL) gene from M. tuberculosis, indicating that this protein is an M. vaccae member of the GroEL family.

An expression library of M. vaccae genomic DNA in BamH1-lambda ZAP-Express (Stratagene) was screened using sera from cynomolgous monkeys immunized with M. tuberculosis secreted proteins prepared as described above. Positive plaques were identified using a colorimetric system. These plaques were re-screened until plaques were pure following standard procedures. pBK-CMV phagemid 2-1 containing an insert was excised from the lambda ZAP-Express (Stratagene) vector in the presence of ExAssist helper phage following the manufacturer's protocol. The base sequence of the 5′ end of the insert of this clone, hereinafter referred to as GV-27, was determined using Sanger sequencing with fluorescent primers on Perkin Elmer/Applied Biosystems Division automatic sequencer. The determined nucleotide sequence of the partial M. vaccae GroEL-homologue clone GV-27 is provided in SEQ ID NO:77 and the predicted amino acid sequence in SEQ ID NO:78. This clone was found to have homology to M. tuberculosis GroEL.

A partial sequence of the 65 kDa heat shock protein of M. vaccae has been published by Kapur et al. (Arch. Pathol. Lab. Med. 119:131-138, 1995). However, this sequence did not overlap with the GV-27 sequence provided herein. The nucleotide sequence of the Kapur et al. fragment is shown in SEQ ID NO:79 and the predicted amino acid sequence in SEQ ID NO:80.

In subsequent studies, an extended DNA sequence (full-length except for the predicted 51 terminal residues) for GV-27 was obtained (SEQ ID NO: 113). The corresponding predicted amino acid sequence is provided in SEQ ID NO: 114. Further studies led to the isolation of the full-length DNA sequence for GV-27 (SEQ ID NO: 159). The corresponding predicted amino acid sequence is provided in SEQ ID NO: 160. This sequence shows 93.7% identity to the M. tuberculosis GroEL sequence. Two peptide fragments, comprising the N-terminal sequence (hereinafter referred to as GV-27A) and the carboxy terminal sequence of GV-27 (hereinafter referred to as GV-27B) were prepared using techniques well known in the art. The nucleotide sequences for GV-27A and GV-27B are provided in SEQ ID NO: 115 and 116, respectively, with the corresponding amino acid sequences being provided in SEQ ID NO: 117 and 118. Subsequent studies led to the isolation of an extended DNA sequence for GV-27B. This sequence is provided in SEQ ID NO: 161, with the corresponding amino acid sequence being provided in SEQ ID NO: 162. The sequence of GV-27A shows 95.8% identity to the published M. tuberculosis GroEL sequence and contains the M. vaccae sequence of Kapur et al. discussed above. The sequence of GV-27B is about 92.2% identical to the published M. tuberculosis sequence.

Following the same protocol as for the isolation of GV-27, pBK-CMV phagemid 3-1 was isolated. The antigen encoded by this DNA was named GV-29. The determined nucleotide sequences of the 5′ and 3′ ends of the gene are provided in SEQ ID NO: 163 and 164, respectively, with the predicted corresponding amino acid sequences being provided in SEQ ID NO: 165 and 166 respectively. GV-29 showed homology to yeast urea amidolyase. The determined DNA sequence for the full-length gene encoding GV-29 is provided in SEQ ID NO: 198, with the corresponding predicted amino acid sequence being provided in SEQ ID NO: 199. The DNA encoding GV-29 was sub-cloned into the vector pET16 (Novagen, Madison, Wis.) for expression and purification according to standard protocols.

### EXAMPLE 5

### Purification and Characterization of Polypeptides from M. vaccae Culture Filtrate

This example illustrates the preparation of M. vaccae soluble proteins from culture filtrate. Unless otherwise noted, all percentages in the following example are weight per volume.

M. vaccae (ATCC Number 15483) was cultured in sterile Medium 90 at 37° C. The cells were harvested by centrifugation, and transferred into sterile Middlebrook 7H9 medium with glucose at 37° C. for one day. The medium was then centrifuged (leaving the bulk of the cells) and filtered through a 0.45 μm filter into sterile bottles.

The culture filtrate was concentrated by lyophilization, and redissolved in MilliQ water. A small amount of insoluble material was removed by filtration through a 0.45 m membrane. The culture filtrate was desalted by membrane filtration in a 400 ml Amicon stirred cell which contained a 3,000 Da molecular weight cut-off (MWCO) membrane. The pressure was maintained at 50 psi using nitrogen gas. The culture filtrate was repeatedly concentrated by membrane filtration and diluted with water until the conductivity of the sample was less than 1.0 mS. This procedure reduced the 20 l volume to approximately 50 ml. Protein concentrations were determined by the Bradford protein assay (Bio-Rad, Hercules, Calif., USA).

The desalted culture filtrate was fractionated by ion exchange chromatography on a column of Q-Sepharose (Pharmacia Biotech, Uppsala, Sweden) (16×100 mm) equilibrated with 10 mM Tris HCl buffer pH 8.0. Polypeptides were eluted with a linear gradient of NaCl from 0 to 1.0 M in the above buffer system. The column eluent was monitored at a wavelength of 280 nm.

The pool of polypeptides eluting from the ion exchange column was concentrated in a 400 ml Amicon stirred cell which contained a 3,000 Da MWCO membrane. The pressure was maintained at 50 psi using nitrogen gas. The polypeptides were repeatedly concentrated by membrane filtration and diluted with 1% glycine until the conductivity of the sample was less than 0.1 mS.

The purified polypeptides were then fractionated by preparative isoelectric focusing in a Rotofor device (Bio-Rad, Hercules, Calif., USA). The pH gradient was established with a mixture of Ampholytes (Pharmacia Biotech) comprising 1.6% pH 3.5-5.0 Ampholytes and 0.4% pH 5.0-7.0 Ampholytes. Acetic acid (0.5 M) was used as the anolyte, and 0.5 M ethanolamine as the catholyte. Isoelectric focusing was carried out at 12 W constant power for 6 hours, following the manufacturer's instructions. Twenty fractions were obtained.

Fractions from isoelectric focusing were combined, and the polypeptides were purified on a Vydac C4 column (Separations Group, Hesperia, Calif., USA) 300 Angstrom pore size, 5 micron particle size (10×250 mm). The polypeptides were eluted from the column with a linear gradient of acetonitrile (0-80% v/v) in 0.05% (v/v) trifluoroacetic acid (TFA). The flow-rate was 2.0 ml/min and the HPLC eluent was monitored at 220 nm. Fractions containing polypeptides were collected to maximize the purity of the individual samples.

Relatively abundant polypeptide fractions were rechromatographed on a Vydac C4 column (Separations Group) 300 Angstrom pore size, 5 micron particle size (4.6×250 mm). The polypeptides were eluted from the column with a linear gradient from 20-60% (v/v) of acetonitrile in 0.05% (v/v) TFA at a flow-rate of 1.0 ml/min. The column eluent was monitored at 220 nm. Fractions containing the eluted polypeptides were collected to maximise the purity of the individual samples. Approximately 20 polypeptide samples were obtained and they were analysed for purity on a polyacrylamide gel according to the procedure of Laemmli (Laemmli, U. K., Nature 277:680-685, 1970).

The polypeptide fractions which were shown to contain significant contamination were further purified using a Mono Q column (Pharmacia Biotech) 10 micron particle size (5×50 mm) or a Vydac Diphenyl column (Separations Group) 300 Angstrom pore size, 5 micron particle size (4.6×250 mm). From a Mono Q column, polypeptides were eluted with a linear gradient from 0-0.5 M NaCl in 10 mM Tris HCl pH 8.0. From a Vydac Diphenyl column, polypeptides were eluted with a linear gradient of acetonitrile (20-60% v/v) in 0.1% TFA. The flow-rate was 1.0 ml/min and the column eluent was monitored at 220 nm for both columns. The polypeptide peak fractions were collected and analysed for purity on a 15% polyacrylamide gel as described above.

For sequencing, the polypeptides were individually dried onto Biobrene™ (Perkin Elmer/Applied BioSystems Division, Foster City, Calif.)-treated glass fiber filters. The filters with polypeptide were loaded onto a Perkin Elmer/Applied BioSystems Procise 492 protein sequencer and the polypeptides were sequenced from the amino terminal end using traditional Edman chemistry. The amino acid sequence was determined for each polypeptide by comparing the retention time of the PTH amino acid derivative to the appropriate PTH derivative standards.

Internal sequences were also determined on some antigens by digesting the antigen with the endoprotease Lys-C, or by chemically cleaving the antigen with cyanogen bromide. Peptides resulting from either of these procedures were separated by reversed-phase HPLC on a Vydac C18 column using a mobile phase of 0.05% (v/v) trifluoroacetic acid with a gradient of acetonitrile containing 0.05% (v/v) TFA (1%/min). The eluent was monitored at 214 nm. Major internal peptides were identified by their UV absorbance, and their N-terminal sequences were determined as described above.

Using the procedures described above, six soluble M. vaccae antigens, designated GVc-1, GVc-2, GVc-7, GVc-13, GVc-20 and GVc-22, were isolated. Determined N-terminal and internal sequences for GVc-1 are shown in SEQ ID NO: 1, 2 and 3, respectively; the N-terminal sequence for GVc-2 is shown in SEQ ID NO: 4; internal sequences for GVc-7 are shown in SEQ ID NO: 5-8; internal sequences for GVc-13 are shown in SEQ ID NO: 9-11; internal sequence for GVc-20 is shown in SEQ ID NO: 12; and N-terminal and internal sequences for GVc-22 are shown in SEQ ID NO:56-59, respectively. Each of the internal peptide sequences provided herein begins with an amino acid residue which is assumed to exist in this position in the polypeptide, based on the known cleavage specificity of cyanogen bromide (Met) or Lys-C (Lys).

Three additional polypeptides, designated GVc-16, GVc-18 and GVc-21, were isolated employing a preparative sodium dodecyl sulfate-polyacrylamide gel electrophoresis (SDS-PAGE) purification step in addition to the preparative isoelectric focusing procedure described above. Specifically, fractions comprising mixtures of polypeptides from the preparative isoelectric focusing purification step previously described, were purified by preparative SDS-PAGE on a 15% polyacrylamide gel. The samples were dissolved in reducing sample buffer and applied to the gel. The separated proteins were transferred to a polyvinylidene difluoride (PVDF) membrane by electroblotting in 10 mM 3-(cyclohexylamino)-1-propanesulfonic acid (CAPS) buffer pH 11 containing 10% (v/v) methanol. The transferred protein bands were identified by staining the PVDF membrane with Coomassie blue. Regions of the PVDF membrane containing the most abundant polypeptide species were cut out and directly introduced into the sample cartridge of the Perkin Elmer/Applied BioSystems Procise 492 protein sequencer. Protein sequences were determined as described above. The N-terminal sequences for GVc-16, GVc-18 and GVc-21 are provided in SEQ ID NO: 13, 14 and 15, respectively.

Additional antigens, designated GVc-12, GVc-14, GVc-15, GVc-17 and GVc-19, were isolated employing a preparative SDS-PAGE purification step in addition to the chromatographic procedures described above. Specifically, fractions comprising a mixture of antigens from the Vydac C4 HPLC purification step previously described were fractionated by preparative SDS-PAGE on a polyacrylamide gel. The samples were dissolved in non-reducing sample buffer and applied to the gel. The separated proteins were transferred to a PVDF membrane by electroblotting in 10 mM CAPS buffer, pH 11 containing 10% (v/v) methanol. The transferred protein bands were identified by staining the PVDF membrane with Coomassie blue. Regions of the PVDF membrane containing the most abundant polypeptide species were cut out and directly introduced into the sample cartridge of the Perkin Elmer/Applied BioSystems Procise 492 protein sequencer. Protein sequences were determined as described above. The determined N-terminal sequences for GVc-12, GVc-14, GVc-15, GVc-17 and GVc-19 are provided in SEQ ID NO: 16-20, respectively.

All of the above amino acid sequences were compared to known amino acid sequences in the SwissProt data base (version R32) using the GeneAssist system. No significant homologies to the amino acid sequences GVc-2 to GVc-22 were obtained. The amino acid sequence for GVc-1 was found to bear some similarity to sequences previously identified from M. bovis and M. tuberculosis. In particular, GVc-1 was found to have some homology with M. tuberculosis MPT83, a cell surface protein, as well as MPT70. These proteins form part of a protein family (Harboe et al., Scand. J. Immunol. 42:46-51, 1995).

Subsequent studies led to the isolation of DNA sequences for GVc-13, GVc-14 and GVc-22 (SEQ ID NO: 142, 107 and 108, respectively). The corresponding predicted amino acid sequences for GVc-13, GVc-14 and GVc-22 are provided in SEQ ID NO: 143, 109 and 110, respectively. The determined DNA sequence for the full length gene encoding GVc-13 is provided in SEQ ID NO: 195, with the corresponding predicted amino acid sequence being provided in SEQ ID NO: 196.

Further studies with GVc-22 suggested that only a part of the gene encoding GVc-22 was cloned. When sub-cloned into the expression vector pET16, no protein expression was obtained. Subsequent screening of the M. vaccae BamHI genomic DNA library with the incomplete gene fragment led to the isolation of the complete gene encoding GVc-22. To distinguish between the full-length clone and the partial GVc-22, the antigen expressed by the full-length gene was called GV-22B. The determined nucleotide sequence of the gene encoding GV-22B and the predicted amino acid sequence are provided in SEQ ID NO: 144 and 145 respectively.

Amplifications primers AD86 and AD112 (SEQ ID NO: 60 and 61, respectively) were designed from the amino acid sequence of GVc-1 (SEQ ID NO: 1) and the M. tuberculosis MPT70 gene sequence. Using these primers, a 310 bp fragment was amplified from M. vaccae genomic DNA and cloned into EcoRV-digested vector pBluescript II SK⁺ (Stratagene). The sequence of the cloned insert is provided in SEQ ID NO: 62. The insert of this clone was used to screen a M. vaccae genomic DNA library constructed in lambda ZAP-Express (Stratgene, La Jolla, Calif.). The clone isolated contained an open reading frame with homology to the M. tuberculosis antigen MPT83 and was re-named GV-1/83. This gene also had homology to the M. bovis antigen MPB83. The determined nucleotide sequence and predicted amino acid sequences are provided in SEQ ID NO: 146 and 147 respectively.

From the amino acid sequences provided in SEQ ID NO: 1 and 2, degenerate oligonucleotides EV59 and EV61 (SEQ ID NO: 148 and 149 respectively) were designed. Using PCR, a 100 bp fragment was amplified, cloned into plasmid pBluescript II SK⁺ and sequenced (SEQ ID NO: 150) following standard procedures (Maniatis). The cloned insert was used to screen a M. vaccae genomic DNA library constructed in lambda ZAP-Express. The clone isolated had homology to M. tuberculosis antigen MPT70 and M. bovis antigen MPB70, and was named GV-1/70. The determined nucleotide sequence and predicted amino acid sequence for GV-1/70 are provided in SEQ ID NO: 151 and 152, respectively.

For expression and purification, the genes encoding GV1/83, GV1/70, GVc-13, GVc-14 and GV-22B were sub-cloned into the expression vector pET16 (Novagen, Madison, Wis.). Expression and purification were carried out according to the manufacturer's protocol.

The purified polypeptides were screened for the ability to induce T-cell proliferation and IFN-γ in peripheral blood cells from immune human donors. These donors were known to be PPD (purified protein derivative from M. tuberculosis) skin test positive and their T cells were shown to proliferate in response to PPD. Donor PBMCs and crude soluble proteins from M. vaccae culture filtrate were cultured in medium comprising RPMI 1640 supplemented with 10% (v/v) autologous serum, penicillin (60 mg/ml), streptomycin (100 mg/ml), and glutamine (2 mM).

After 3 days, 50 μl of medium was removed from each well for the determination of IFN-γ levels, as described below. The plates were cultured for a further 4 days and then pulsed with 1 mCi/well of tritiated thymidine for a further 18 hours, harvested and tritium uptake determined using a scintillation counter. Fractions that stimulated proliferation in both replicates two-fold greater than the proliferation observed in cells cultured in medium alone were considered positive.

IFN-γ was measured using an enzyme-linked immunosorbent assay (ELISA). ELISA plates were coated with a mouse monoclonal antibody directed to human IFN-gamma (Endogen, Wobural, Mass.) 1 mg/ml phosphate-buffered saline (PBS) for 4 hours at 4° C. Wells were blocked with PBS containing 0.2% Tween 20 for 1 hour at room temperature. The plates were then washed four times in PBS/0.2% Tween 20, and samples diluted 1:2 in culture medium in the ELISA plates were incubated overnight at room temperature. The plates were again washed, and a biotinylated polyclonal rabbit anti-human IFN-γ serum (Endogen), diluted to 1 mg/ml in PBS, was added to each well. The plates were then incubated for 1 hour at room temperature, washed, and horseradish peroxidase-coupled avidin A (Vector Laboratories, Burlingame, Calif.) was added at a 1:4,000 dilution in PBS. After a further 1 hour incubation at room temperature, the plates were washed and orthophenylenediamine (OPD) substrate added. The reaction was stopped after 10 min with 10% (v/v) HCl. The optical density (OD) was determined at 490 nm. Fractions that resulted in both replicates giving an OD two-fold greater than the mean OD from cells cultured in medium alone were considered positive.

Examples of polypeptides containing sequences that stimulate peripheral blood mononuclear cells (PBMC) T cells to proliferate and produce IFN-γ are shown in Table 8, wherein (−) indicates a lack of activity, (±) indicates polypeptides having a result less than twice higher than background activity of control media, (+) indicates polypeptides having activity two to four times above background, and (++) indicates polypeptides having activity greater than four times above background.

TABLE 8
Examples of Polypeptides Stimulating Human
Peripheral Blood Mononuclear Cells

| Antigen | Proliferation | IFN-γ |
|---|---|---|
| GVc-1 | ++ | +/− |
| GVc-2 | + | ++ |
| GVc-7 | +/− | − |
| GVc-13 | + | ++ |
| GVc-14 | ++ | + |
| GVc-15 | + | + |
| GVc-20 | + | + |

### EXAMPLE 6

### Purification and Characterisation of Polypeptides from M. vaccae Culture Filtrate by 2-Dimensional Polyacrylamide Gel Electrophoresis

M. vaccae soluble proteins were isolated from culture filtrate using 2-dimensional polyacrylamide gel electrophoresis as described below. Unless otherwise noted, all percentages in the following example are weight per volume.

M. vaccae (ATCC Number 15483) was cultured in sterile Medium 90 at 37° C. M. tuberculosis strain H37Rv (ATCC number 27294) was cultured in sterile Middlebrook 7H9 medium with Tween 80 and oleic acid/albumin/dextrose/catalase additive (Difco Laboratories, Detroit, Mich.). The cells were harvested by centrifugation, and transferred into sterile Middlebrook 7H9 medium with glucose at 37° C. for one day. The medium was then centrifuged (leaving the bulk of the cells) and filtered through a 0.45 μm filter into sterile bottles. The culture filtrate was concentrated by lyophilization, and redissolved in MilliQ water. A small amount of insoluble material was removed by filtration through a 0.45 μm membrane filter.

The culture filtrate was desalted by membrane filtration in a 400 ml Amicon stirred cell which contained a 3,000 Da MWCO membrane. The pressure was maintained at 60 psi using nitrogen gas. The culture filtrate was repeatedly concentrated by membrane filtration and diluted with water until the conductivity of the sample was less than 1.0 mS. This procedure reduced the 20 l volume to approximately 50 ml. Protein concentrations were determined by the Bradford protein assay (Bio-Rad, Hercules, Calif., USA).

The desalted culture filtrate was fractionated by ion exchange chromatography on a column of Q-Sepharose (Pharmacia Biotech) (16×100 mm) equilibrated with 10 M TrisHCl buffer pH 8.0. Polypeptides were eluted with a linear gradient of NaCl from 0 to 1.0 M in the above buffer system. The column eluent was monitored at a wavelength of 280 nm.

The pool of polypeptides eluting from the ion exchange column were fractionated by preparative 2-D gel electrophoresis. Samples containing 200-500 μg of polypeptide were made 8M in urea and applied to polyacrylamide isoelectric focusing rod gels (diameter 2 mm, length 150 mm, pH 5-7). After the isoelectric focusing step, the first dimension gels were equilibrated with reducing buffer and applied to second dimension gels (16% polyacrylamide). FIGS. 4A and 4B are the 2-D gel patterns observed with M. vaccae culture filtrate and M. tuberculosis H37Rv culture filtrate, respectively. Polypeptides from the second dimension separation were transferred to PVDF membranes by electroblotting in 10 mM CAPS buffer pH 11 containing 10% (v/v) methanol. The PVDF membranes were stained for protein with Coomassie blue. Regions of PVDF containing polypeptides of interest were cut out and directly introduced into the sample cartridge of the Perkin Elmer/Applied BioSystems Procise 492 protein sequencer. The polypeptides were sequenced from the amino terminal end using traditional Edman chemistry. The amino acid sequence was determined for each polypeptide by comparing the retention time of the PTH amino acid derivative to the appropriate PTH derivative standards. Using these procedures, eleven polypeptides, designated GVs-1, GVs-3, GVs-4, GVs-5, GVs-6, GVs-8, GVs-9, GVs-10, GVs-11, GV-34 and GV-35 were isolated. The determined N-terminal sequences for these polypeptides are shown in SEQ ID NO: 21-29, 63 and 64, respectively. Using the purification procedure described above, more protein was purified to extend the amino acid sequence previously obtained for GVs-9. The extended amino acid sequence for GVs-9 is provided in SEQ ID NO:65. Further studies resulted in the isolation of the DNA sequences for GVs-9 (SEQ ID NO: 111) and GV-35 (SEQ ID NO: 155). The corresponding predicted amino acid sequences are provided in SEQ ID NO: 112 and 156, respectively. An extended DNA sequence for GVs-9 is provided in SEQ ID NO: 153, with the corresponding predicted amino acid sequence being provided in SEQ ID NO: 154. The predicted amino acid sequence for GVs-9 has been amended in SEQ ID NO: 197.

All of these amino acid sequences were compared to known amino acid sequences in the SwissProt data base (version R32) using the GeneAssist system. No significant homologies were obtained, with the exceptions of GVs-3, GVs-4, GVs-5 and GVs-9. GVs-9 was found to bear some homology to two previously identified M. tuberculosis proteins, namely M. tuberculosis cutinase precursor and a M. tuberculosis hypothetical 22.6 kDa protein. GVs-3, GVs-4 and GVs-5 were found to bear some similarity to the antigen 85A and 85B proteins from M. leprae (SEQ ID NO: 30 and 31, respectively), M. tuberculosis (SEQ ID NO: 32 and 33, respectively) and M. bovis (SEQ ID NO: 34 and 35, respectively), and the antigen 85C proteins from M. leprae (SEQ ID NO: 36) and M. tuberculosis (SEQ ID NO: 37).

### EXAMPLE 7

### DNA Cloning Strategy for the M. Vaccae Antigen 85 Series

Probes for antigens 85A, 85B, and 85C were prepared by the polymerase chain reaction (PCR) using degenerate oligonucleotides (SEQ ID NO: 38 and 39) designed to regions of antigen 85 genomic sequence that are conserved between family members in a given mycobacterial species, and between mycobacterial species. These oligonucleotides were used under reduced stringency conditions to amplify target sequences from M. vaccae genomic DNA. An appropriately-sized 485 bp band was identified, purified, and cloned pBluescript II SK⁺0 (Stratagene, La Jolla, Calif.). Twenty-four individual colonies were screened at random for the presence of the antigen 85 PCR product, then sequenced using the Perkin Elmer/Applied Biosystems Model 377 automated sequencer and the M13-based primers, T3 and T7. Homology searches of the GenBank databases showed that twenty-three clones contained insert with significant homology to published antigen 85 genes from M. tuberculosis and M. bovis. Approximately half were most homologous to antigen 85C gene sequences, with the remainder being more similar to antigen 85B sequences. In addition, these two putative M. vaccae antigen 85 genomic sequences were 80% homologous to one another. Because of this high similarity, the antigen 85C PCR fragment was chosen to screen M. vaccae genomic libraries at low stringency for all three antigen 85 genes.

An M. vaccae genomic library was created in lambda Zap-Express (Stratagene, La Jolla, Calif.) by cloning BamHI partially-digested M. vaccae genomic DNA into similarly-digested vector, with 3.4×10⁵ independent plaque-forming units resulting. For screening purposes, twenty-seven thousand plaques from this non-amplified library were plated at low density onto eight 100 cm² plates. For each plate, duplicate plaque lifts were taken onto Hybond-N⁺ nylon membrane (Amersham International, United Kingdom), and hybridised under reduced-stringency conditions (55° C.) to the radiolabelled antigen 85C PCR product. Autoradiography demonstrated that seventy-nine plaques consistently hybridised to the antigen 85C probe under these conditions. Thirteen positively-hybridising plaques were selected at random for further analysis and removed from the library plates, with each positive clone being used to generate secondary screening plates containing about two hundred plaques. Duplicate lifts of each plate were taken using Hybond-N⁺ nylon membrane, and hybridised under the conditions used in primary screening. Multiple positively-hybridising plaques were identified on each of the thirteen plates screened. Two well-isolated positive phage from each secondary plate were picked for further analysis. Using in vitro excision, twenty-six plaques were converted into phagemid, and restriction-mapped. It was possible to group clones into four classes on the basis of this mapping. Sequence data from the 5′ and 3′ ends of inserts from several representatives of each group was obtained using the Perkin Elmer/Applied Biosystems Division Model 377 automated sequencer and the T3 and T7 primers. Sequence homologies were determined using FASTA analysis of the GenBank databases with the GeneAssist software package. Two of these sets of clones were found to be homologous to M. bovis and M. tuberculosis antigen 85A genes, each containing either the 5′ or 3′ ends of the M. vaccae gene (this gene was cleaved during library construction as it contains an internal BamHI site). The remaining clones were found to contain sequences homologous to antigens 85B and 85C from a number of mycobacterial species. To determine the remaining nucleotide sequence for each gene, appropriate subclones were constructed and sequenced. Overlapping sequences were aligned using the DNA Strider software. The determined DNA sequences for M. vaccae antigens 85A, 85B and 85C are shown in SEQ ID NO: 40-42, respectively, with the predicted amino acid sequences being shown in SEQ ID NO: 43-45, respectively.

The M. vaccae antigens GVs-3 and GVs-5 were expressed and purified as follows. Amplification primers were designed from the insert sequences of GVs-3 and GVs-5 (SEQ ID NO: 40 and 42, respectively) using sequence data downstream from the putative leader sequence and the 3′ end of the clone. The sequences of the primers for GVs-3 are provided in SEQ ID NO: 66 and 67, and the sequences of the primers for GVs-5 are provided in SEQ ID NO: 68 and 69. A XhoI restriction site was added to the primers for GVs-3, and EcoRI and BamHI restriction sites were added to the primers for GVs-5 for cloning convenience. Following amplification from genomic M. vaccae DNA, fragments were cloned into the appropriate site of pProEX HT prokaryotic expression vector (Gibco BRL, Life Technologies, Gaithersburg, Md.) and submitted for sequencing to confirm the correct reading frame and orientation. Expression and purification of the recombinant protein was performed according to the manufacturer's protocol.

Expression of a fragment of the M. vaccae antigen GVs-4 (antigen 85B homolog) was performed as follows. The primers AD58 and AD59, described above, were used to amplify a 485 bp fragment from M. vaccae genomic DNA. This fragment was gel-purified using standard techniques and cloned into EcoRV-digested pBluescript. The base sequences of inserts from five clones were determined and found to be identical to each other. These inserts had highest homology to Ag85B from M. tuberculosis. The insert from one of the clones was subcloned into the EcoRI/XhoI sites of pProEX HT prokaryotic expression vector (Gibco BRL), expressed and purified according to the manufacturer's protocol. This clone was renamed GV-4P because only a part of the gene was expressed. The amino acid and DNA sequences for the partial clone GV-4P are provided in SEQ ID NO: 70 and 106, respectively.

Similar to the cloning of GV-4P, the amplification primers AD58 and AD59 were used to amplify a 485 bp fragment from a clone containing GVs-5 (SEQ ID NO:42). This fragment was cloned into the expression vector pET16 and was called GV-5P. The determined nucleotide sequence and predicted amino acid sequence of GV-5P are provided in SEQ ID NO: 157 and 158, respectively.

The ability of purified recombinant GVs-3, GV-4P and GVs-5 to stimulate proliferation of T cells and interferon-γ production in human PBL was assayed as described above. The results of this assay are shown in Table 9, wherein (−) indicates a lack of activity, (±) indicates polypeptides having a result less than twice higher than background activity of control media, (+) indicates polypeptides having activity two to four times above background, (++) indicates polypeptides having activity greater than four times above background, and ND indicates not determined.

TABLE 9

| | Donor | | Donor | | Donor | | Donor | | Donor | | Donor | |
|---|---|---|---|---|---|---|---|---|---|---|---|---|
| | G97005 | | G97006 | | G97007 | | G97008 | | G97009 | | G97010 | |
| | Prolif | IFN-g | Prolif | IFN-g | Prolif | IFN-g | Prolif | IFN-g | Prolif | IFN-g | Prolif | IFN-g |
| GVs-3 | ++ | + | ND | ND | ++ | ++ | ++ | ++ | ++ | +/− | + | ++ |
| GV-4P | + | +/− | ND | ND | + | ++ | ++ | ++ | +/− | +/− | +/− | ++ |
| GVs-5 | ++ | ++ | ++ | ++ | ++ | ++ | + | ++ | ++ | + | + | ++ |

### EXAMPLE 8

### DNA Cloning Strategy for M. vaccae Antigens

An 84 bp probe for the M. vaccae antigen GVc-7 was amplified using degenerate oligonucleotides designed to the determined amino acid sequence of GVc-7 (SEQ ID NO: 5-8). This probe was used to screen a M. vaccae genomic DNA library as described in Example 4. The determined nucleotide sequence for GVc-7 is shown in SEQ ID NO: 46 and predicted amino acid sequence in SEQ ID NO: 47. Comparison of these sequences with those in the databank revealed homology to a hypothetical 15.8 kDa membrane protein of M. tuberculosis.

The sequence of SEQ ID NO: 46 was used to design amplification primers (provided in SEQ ID NO: 71 and 72) for expression cloning of the GVc-7 gene using sequence data downstream from the putative leader sequence. A XhoI restriction site was added to the primers for cloning convenience. Following amplification from genomic M. vaccae DNA, fragments were cloned into the XhoI-site of pProEX HT prokaryotic expression vector (Gibco BRL) and submitted for sequencing to confirm the correct reading frame and orientation. Expression and purification of the fusion protein was performed according to the manufacturer's protocol.

The ability of purified recombinant GVc-7 to stimulate proliferation of T-cells and stimulation of interferon-γ production in human PBL was assayed as described above. The results are shown in Table 10, wherein (−) indicates a lack of activity, (±) indicates polypeptides having a result less than twice higher than background activity of control media, (+) indicates polypeptides having activity two to four times above background, and (++) indicates polypeptides having activity greater than four times above background.

TABLE 10

| Donor | Proliferation | Interferon-γ |
|---|---|---|
| G97005 | ++ | +/− |
| G97008 | ++ | + |
| G97009 | + | +/− |
| G97010 | +/− | ++ |

A redundant oligonucleotide probe (SEQ ID NO: 73, referred to as MPG15) was designed to the GVs-8 peptide sequence shown in SEQ ID NO: 26 and used to screen a M. vaccae genomic DNA library using standard protocols. A genomic clone containing genes encoding four different antigens was isolated. The determined DNA sequences for GVs-8A (re-named GV-30), GVs-8B (re-named GV-31), GVs-8C (re-named GV-32) and GVs-8D, (re-named GV-33) are shown in SEQ ID NO: 48-51, respectively, with the corresponding amino acid sequences being shown in SEQ ID NO: 52-55, respectively. GV-30 contains regions showing some similarity to known prokaryotic valyl-tRNA synthetases; GV-31 shows some similarity to M. smegmatis aspartate semialdehyde dehydrogenase; and GV-32 shows some similarity to the H. influenza folylpolyglutamate synthase gene. GV-33 contains an open reading frame which shows some similarity to sequences previously identified in M. tuberculosis and M. leprae, but whose function has not been identified.

The determined partial DNA sequence for GV-33 is provided in SEQ ID NO:74 with the corresponding predicted amino acid sequence being provided in SEQ ID NO:75. Sequence data from the 3′ end of the clone showed homology to a previously identified 40.6 kDa outer membrane protein of M. tuberculosis. Subsequent studies led to the isolation of the full-length DNA sequence for GV-33 (SEQ ID NO: 193). The corresponding predicted amino acid sequence is provided in SEQ ID NO: 194.

The gene encoding GV-33 was amplified from M. vaccae genomic DNA with primers based on the determined nucleotide sequence. This DNA fragment was cloned into EcoRv-digested pBluescript II SK⁺ (Stratagene), and then transferred to pET16 expression vector. Recombinant protein was purified following the manufacturer's protocol.

The ability of purified recombinant GV-33 to stimulate proliferation of T-cells and stimulation of interferon-γ production in human PBL was assayed as described above. The results are shown in Table 11, wherein (−) indicates a lack of activity, (±) indicates polypeptides having a result less than twice higher than background activity of control media, (+) indicates polypeptides having activity two to four times above background, and (++) indicates polypeptides having activity greater than four times above background.

TABLE 11
Stimulatory Activity of Polypeptides

| Donor | Proliferation | Interferon-γ |
|---|---|---|
| G97005 | ++ | + |
| G97006 | ++ | ++ |
| G97007 | − | +/− |
| G97008 | +/− | − |
| G97009 | +/− | − |
| G97010 | +/− | ++ |

### EXAMPLE 9

### DNA Cloning Strategy for the M. Vaccae Antigens GV-23, GV-24, GV-25, GV-26, GV-38A and GV-38B

M. vaccae (ATCC Number 15483) was grown in sterile Medium 90 at 37° C. for 4 days and harvested by centrifugation. Cells were resuspended in 1 ml Trizol (Gibco BRL, Life Technologies, Gaithersburg, Md.) and RNA extracted according to the standard manufacturer's protocol. M. tuberculosis strain H37Rv (ATCC Number 27294) was grown in sterile Middlebrooke 7H9 medium with Tween 80™ and oleic acid/ albumin/dextrose/catalase additive (Difco Laboratories, Detroit, Mich.) at 37° C. and harvested under appropriate laboratory safety conditions. Cells were resuspended in 1 ml Trizol (Gibco BRL) and RNA extracted according to the manufacturer's standard protocol.

Total M. tuberculosis and M. vaccae RNA was depleted of 16S and 23S ribosomal RNA (rRNA) by hybridization of the total RNA fraction to oligonucleotides AD10 and AD11 (SEQ ID NO: 81 and 82) complementary to M. tuberculosis rRNA. These oligonucleotides were designed from mycobacterial 16S rRNA sequences published by Bottger (FEMS Microbiol. Lett. 65:171-176, 1989) and from sequences deposited in the databanks. Depletion was done by hybridisation of total RNA to oligonucleotides AD10 and AD11 immobilised on nylon membranes (Hybond N, Amersham International, United Kingdom). Hybridization was repeated until rRNA bands were not visible on ethidium bromide-stained agarose gels. An oligonucleotide, AD12 (SEQ ID NO: 83), consisting of 20 dATP-residues, was ligated to the 3′ ends of the enriched mRNA fraction using RNA ligase. First strand cDNA synthesis was performed following standard protocols, using oligonucleotide AD7 (SEQ ID NO:84) containing a poly(dT) sequence.

The M. tuberculosis and M. vaccae cDNA was used as template for single-sided-specific PCR (3S-PCR). For this protocol, a degenerate oligonucleotide AD1 (SEQ ID NO:85) was designed based on conserved leader sequences and membrane protein sequences. After 30 cycles of amplification using primer AD1 as 5′-primer and AD7 as 3′-primer, products were separated on a urea/polyacrylamide gel. DNA bands unique to M. vaccae were excised and re-amplified using primers AD1 and AD7. After gel purification, bands were cloned into pGEM-T (Promega) and the base sequence determined.

Searches with the determined nucleotide and predicted amino acid sequences of band 12B21 (SEQ ID NO: 86 and 87, respectively) showed homology to the pota gene of E.coli encoding the ATP-binding protein of the spermidine/putrescine ABC transporter complex published by Furuchi et al. (Jnl. Biol. Chem. 266: 20928-20933, 1991). The spermidine/putrescine transporter complex of E.coli consists of four genes and is a member of the ABC transporter family. The ABC (ATP-binding Cassette) transporters typically consist of four genes: an ATP-binding gene, a periplasmic, or substrate binding, gene and two transmembrane genes. The transmembrane genes encode proteins each characteristically having six membrane-spanning regions. Homologues (by similarity) of this ABC transporter have been identified in the genomes of Haemophilus influenza (Fleischmann et al. Science 269:496-512, 1995) and Mycoplasma genitalium (Fraser, et al. Science, 270:397-403, 1995).

A M. vaccae genomic DNA library constructed in BamHI-digested lambda ZAP Express (Stratagene) was probed with the radiolabelled 238 bp band 12B21 following standard protocols. A plaque was purified to purity by repetitive screening and a phagemid containing a 4.5 kb insert was identified by Southern blotting and hybridisation. The nucleotide sequence of the full-length M. vaccae homologue of pota (ATP-binding protein) was identified by subcloning of the 4.5 kb fragment and base sequencing. The gene consisted of 1449 bp including an untranslated 5′ region of 320 bp containing putative −10 and −35 promoter elements. The nucleotide and predicted amino acid sequences of the M. vaccae pota homologue are provided in SEQ ID NO: 88 and 89, respectively.

The nucleotide sequence of the M. vaccae pota gene was used to design primers EV24 and EV25 (SEQ ID NO: 90 and 91) for expression cloning. The amplified DNA fragment was cloned into pProEX HT prokaryotic expression system (Gibco BRL) and expression in an appropriate E.coli host was induced by addition of 0.6 mM isopropylthio-β-galactoside (IPTG). The recombinant protein was named GV-23 and purified from inclusion bodies according to the manufacturer's protocol.

A 322 bp Sal1-BamH1 subclone at the 3′-end of the 4.5 kb insert described above showed homology to the potd gene, (periplasmic protein), of the spermidine/putrescine ABC transporter complex of E. coli. The nucleotide sequence of this subclone is shown in SEQ ID NO:92. To identify the gene, the radiolabelled insert of this subclone was used to probe an M. vaccae genomic DNA library constructed in the Sal1-site of lambda Zap-Express (Stratagene) following standard protocols. A clone was identified of which 1342 bp showed homology with the potd gene of E. coli. The potd homologue of M. vaccae was identified by sub-cloning and base sequencing. The determined nucleotide and predicted amino acid sequences are shown in SEQ ID NO: 93 and 94.

For expression cloning, primers EV26 and EV27 (SEQ ID NO:95-96) were designed from the determined M. vaccae potd homologue. The amplified fragment was cloned into pProEX HT Prokaryotic expression system (Gibco BRL). Expression in an appropriate E. coli host was induced by addition of 0.6 mM IPTG and the recombinant protein named GV-24. The recombinant antigen was purified from inclusion bodies according to the protocol of the supplier.

To improve the solubility of the purified recombinant antigen, the gene encoding GV-24, but excluding the signal peptide, was re-cloned into the expression vector, employing. amplification primers EV101 and EV102 (SEQ ID NO: 167 and 168). The construct was designated GV-24B. The nucleotide sequence of GV-24B is provided in SEQ ID NO: 169 and the predicted amino acid sequence in SEQ ID NO: 170. This fragment was cloned into pET16 for expression and purification of GV-24B according to the manufacturer's protocols.

The ability of purified recombinant protein GV-23 and GV-24 to stimulate proliferation of T cells and interferon-production in human PBL was determined as described above. The results of these assays are provided in Table 12, wherein (−) indicates a lack of activity, (±) indicates polypeptides having a result less than twice higher than background activity of control media, indicates polypeptides having activity two to four times above background, (++) indicates polypeptides having activity greater than four times above background, and (ND) indicates not determined.

TABLE 12

| | Donor | | Donor | | Donor | | Donor | | Donor | | Donor | |
|---|---|---|---|---|---|---|---|---|---|---|---|---|
| | G97005 | | G97006 | | G97007 | | G97008 | | G97009 | | G97010 | |
| | Prolif | IFN-g | Prolif | IFN-g | Prolif | IFN-γ-g | Prolif | IFN-g | Prolif | IFN-g | Prolif | IFN-g |
| GV-23 | ++ | ++ | ++ | ++ | + | + | ++ | ++ | + | − | + | ++ |
| GV-24 | ++ | + | ++ | + | ND | ND | + | +/− | + | +/− | +/− | ++ |

Base sequence adjacent to the M. vaccae potd gene-homologue was found to show homology to the potb gene of the spermidine/putrescine ABC transporter complex of E.coli, which is one of two transmembrane proteins in the ABC transporter complex. The M. vaccae potb homologue (referred to as GV-25) was identified through further subcloning and base sequencing. The determined nucleotide and predicted amino acid sequences for GV-25 are shown in SEQ ID NO: 97 and 98, respectively.

Further subcloning and base sequence analysis of the adjacent 509 bp failed to reveal significant homology to PotC, the second transmembrane protein of E.coli, and suggests that a second transmembrane protein is absent in the M. vaccae homologue of the ABC transporter. An open reading frame with homology to M. tuberculosis acetyl-CoA acetyl transferase, however, was identified starting 530 bp downstream of the transmembrane protein and the translated protein was named GV-26. The determined partial nucleotide sequence and predicted amino acid sequence for GV-26 are shown in SEQ ID NO: 99 and 100.

Using a protocol similar to that described above for the isolation of GV-23, the 3S-PCR band 12B28 (SEQ ID NO: 119) was used to screen the M. vaccae genomic library constructed in the BamHI-site of lambda ZAP-Express (Stratagene). The clone isolated from the library contained a novel open reading frame and the antigen encoded by this gene was named GV-38A. The determined nucleotide sequence and predicted amino acid sequence of GV-38A are shown in SEQ ID NO: 120 and 121, respectively. Subsequent studies led to the isolation of an extended DNA sequence for GV-38A, provided in SEQ ID NO: 171. The corresponding amino acid sequence is provided in SEQ ID NO: 172. Comparison of these sequences with those in the databases revealed only a limited amount of homology to an unknown M. tuberculosis protein previously identified in cosmid MTCY428.12.

Upstream of the GV-38A gene, a second novel open reading frame was identified and the antigen encoded by this gene was named GV-38B. The determined 5′ and 3′ nucleotide sequences for GV-38B are provided in SEQ ID NO: 122 and 123, respectively, with the corresponding predicted amino acid sequences being provided in SEQ ID NO: 124 and 125, respectively. Further studies led to the isolation of the full-length DNA sequence for GV-38B, provided in SEQ ID NO: 173. The corresponding amino acid sequence is provided in SEQ ID NO: 174. This protein was found to show only a limited amount of homology to an unknown M. tuberculosis protein identified as a putative open reading frame in cosmid MTCY428.11 (SPTREMBL: P71914).

Both the GV-38A and GV-38B antigens were amplified for expression cloning into pET16 (Novagen). GV-38A was amplified with primers KR11 and KR12 (SEQ ID NO: 126 and 127) and GV-38B with primers KR13 and KR14 (SEQ ID NO: 128 and 129). Protein expression in the host cells BL21(DE3) was induced with 1 mM IPTG, however no protein expression was obtained from these constructs. Hydrophobic regions were identified in the N-termini of antigens GV-38A and GV-38B which may inhibit expression of these constructs. The hydrophobic region present in GV-38A was identified as a possible transmembrane motif with six membrane spanning regions. To express the antigens without the hydrophobic regions, primers KR20 for GV-38A, (SEQ ID NO: 130) and KR21 for GV-38B (SEQ ID NO: 131) were designed. The truncated GV-38A gene was amplified with primers KR20 and KR12, and the truncated GV-38B gene with KR21 and KR14. The determined nucleotide sequences of truncated GV-38A and GV-38B are shown in SEQ ID NO: 132 and 133, respectively, with the corresponding predicted amino acid sequences being shown in SEQ ID NO: 134 and 135, respectively. Extended DNA sequences for truncated GV-38A and GV-38B are provided in SEQ ID NO: 175 and 176, respectively, with the corresponding amino acid sequences being provided in SEQ ID NO: 177 and 178, respectively.

### EXAMPLE 10

### Purification and Characterisation of Polypeptides from M. vaccae Culture Filtrate by Preparative Isoelectric Focusing and Preparative Polyacrylamide Gel Electrophoresis

M. vaccae soluble proteins were isolated from culture filtrate using preparative isoelectric focusing and preparative polyacrylamide gel electrophoresis as described below. Unless otherwise noted, all percentages in the following example are weight per volume.

M. vaccae (ATCC Number 15483) was cultured in 250 l sterile Medium 90 which had been fractionated by ultrafiltration to remove all proteins of greater than 10 kDa molecular weight. The medium was centrifuged to remove the bacteria, and sterilised by filtration through a 0.45μ filter. The sterile filtrate was concentrated by ultrafiltration over a 10 kDa molecular weight cut-off membrane.

Proteins were isolated from the concentrated culture filtrate by precipitation with 10% trichloroacetic acid. The precipitated proteins were re-dissolved in 100 mM Tris.HCl pH 8.0 and re-precipitated by the addition of an equal volume of acetone. The acetone precipitate was dissolved in water, and proteins were re-precipitated by the addition of an equal volume of chloroform:methanol 2:1 (v/v). The chloroform:methanol precipitate was dissolved in water, and the solution was freeze-dried.

The freeze-dried protein was dissolved in iso-electric focusing buffer, containing 8 M deionised urea, 2% Triton X-100, 10 mM dithiothreitol and 2% ampholytes (pH 2.5-5.0). The sample was fractionated by preparative iso-electric focusing on a horizontal bed of Ultrodex gel at 8 watts constant power for 16 hours. Proteins were eluted from the gel bed fractions with water and concentrated by precipitation with 10% trichloroacetic acid.

Pools of fractions containing proteins of interest were identified by analytical polyacrylamide gel electrophoresis and fractionated by preparative polyacrylamide gel electrophoresis. Samples were fractionated on 12.5% SDS-PAGE gels, and electroblotted onto nitrocellulose membranes. Proteins were located on the membranes by staining with Ponceau Red, destained with water and eluted from the membranes with 40% acetonitrile/0.1M ammonium bicarbonate pH 8.9 and then concentrated by lyophilization.

Eluted proteins were assayed for their ability to induce proliferation and interferon-γ secretion from the peripheral blood lymphocytes of immune donors as detailed in Example 4. Proteins inducing a strong response in these assays were selected for further study.

Selected proteins were further purified by reversed-phase chromatography on a Vydac Protein C4 column, using a trifluoroacetic acid-acetonitrile system. Purified proteins were prepared for protein sequence determination by SDS-polyacrylamide gel electrophoresis, and electroblotted onto PVDF membranes. Protein sequences were determined as in Example 5. The proteins were named GV-40, GV-41, GV-42, GV-43 and GV-44. The determined N-terminal sequences for these polypeptides are shown in SEQ ID NO:101-105, respectively. Subsequent studies led to the isolation of a 5′, middle fragment and 3′ DNA sequence for GV-42 (SEQ ID NO: 136, 137 and 138, respectively). The corresponding predicted amino acid sequences are provided in SEQ ID NO: 139, 140 and 141, respectively.

Following standard DNA amplification and cloning procedures as described in Example 7, the genes encoding GV-41 and GV-42 were cloned. The determined nucleotide sequences are provided in SEQ ID NO: 179 and 180, respectively, and the predicted amino acid sequences in SEQ ID NO: 181 and 182. Further experiments lead to the cloning of the full-length gene encoding GV-41, which was named GV-41B. The determined nucleotide sequence and the predicted amino acid sequence of GV-41B are provided in SEQ ID NO: 202 and 203, respectively. GV-41 had homology to the ribosome recycling factor of M. tuberculosis and M. leprae, and GV-42 had homology to a M. avium fibronectin attachment protein FAP-A. Within the full-length sequence of GV-42, the amino acid sequence determined for GV-43 (SEQ ID NO: 104) was identified, indicating that the amino acid sequences for GV-42 and GV-43 were obtained from the same protein.

Murine polyclonal antisera were prepared against GV-40 and GV-44 following standard procedures. These antisera were used to screen a M. vaccae genomic DNA library consisting of randomly sheared DNA fragments. Clones encoding GV-40 and GV-44 were identified and sequenced. The determined nucleotide sequence of the partial gene encoding GV-40 is provided in SEQ ID NO: 183 and the predicted amino acid sequence in SEQ ID NO: 184. The complete gene encoding GV-40 was not cloned, and the antigen encoded by this partial gene was named GV-40P. An extended DNA sequence for GV-40P is provided in SEQ ID NO: 206 with the corresponding predicted amino acid sequence being provided in SEQ ID NO: 207. The nucleotide sequence of the gene encoding GV-44 is provided in SEQ ID NO: 185, and the predicted amino acid sequence in SEQ ID NO:186. With further sequencing, the determined DNA sequence for the full-length gene encoding GV-44 was obtained and is provided in SEQ ID NO: 204, with the corresponding predicted amino acid sequence being provided in SEQ ID NO: 205. Homology of GV-40 to M. leprae Elongation factor G was found. GV-44 had homology to M. leprae glyceraldehyde-3-phosphate dehydrogenase.

### EXAMPLE 11

### DNA Cloning Strategy for the DD-M. vaccae Antigen GV-45

Proteins were extracted from DD-M. vaccae (500 mg; prepared as described above) by suspension in 10 ml 2% SDS/PBS and heating to 50° C. for 2 h. The insoluble residue was removed by centrifugation, and proteins precipitated from the supernatant by adding an equal volume of acetone and incubating at −20° C. for 1 hr. The precipitated proteins were collected by centrifugation, dissolved in reducing sample buffer, and fractionated by preparative SDS-polyacrylamide gel electrophoresis. The separated proteins were electroblotted onto PVDF membrane in 10 mM CAPS/0.01% SDS pH 11.0, and N-terminal sequences were determined in a gas-phase sequenator.

The amino acid sequence obtained from these experiments was designated GV-45. The determined N-terminal sequence for GV-45 is provided in SEQ ID NO: 187. From the same experiments, a protein of approximate molecular weight of 14 kDa, designated GV-46, was obtained. The determined N-terminal amino acid sequence of GV-46 is provided in SEQ ID NO: 208. GV 46 is homologous to the highly conserved mycobacterial host integration factor of M. tuberculosis and M. smegmatis.

From the amino acid sequence of GV-45, degenerate oligonucleotides KR32 and KR33 (SEQ ID NO: 188 and 189, respectively) were designed. A 100 bp fragment was amplified, cloned into plasmid pBluescript II SK⁺ (Stratagene, La Jolla, Calif.) and sequenced (SEQ ID NO:190) following standard procedures (Maniatis). The cloned insert was used to screen a M. vaccae genomic DNA library constructed in the BamHI-site of lambda ZAP-Express (Stratagene). The isolated clone showed homology to a 35 kDa M. tuberculosis and a 22 kDa M. leprae protein containing bacterial histone-like motifs at the N-terminus and a unique C-terminus consisting of a five amino acid basic repeat. The determined nucleotide sequence for GV-45 is provided in SEQ ID NO: 191, with the corresponding predicted amino acid sequence being provided in SEQ ID NO: 192. With additional sequencing, the determined DNA sequence for the full-length gene encoding GV-45 was obtained and is provided in SEQ ID NO: 200, with the corresponding predicted amino acid sequence being provided in SEQ ID NO: 201.

Although the present invention has been described in some detail by way of illustration and example for purposes of clarity of understanding, changes and modifications can be carried out without departing from the scope of the invention which is intended to be limited only by the scope of the claims.

We claim:

1. A method for the treatment of asthma in a patient, comprising administering to the patient a composition comprising delipidated and deglycolipidated M. vaccae cells, wherein the delipidated and deglycolipidated M. vaccae cells comprise less than 10% by weight of lipids.

2. The method of claim 1, wherein the composition is administered intranasally.

3. The method of claim 1, wherein the composition is administered subcutaneously.

4. The method of claim 1, wherein the composition additionally comprises an adjuvant.

5. A method for the treatment of asthma in a patient, comprising administering to the patient a composition comprising delipidated and deglycolipidated M. vaccae cells, wherein the delipidated and deglycolipidated M. vaccae cells comprise more than 33% by weight of amino acids.

6. The method of claim 5, wherein the composition is administered intranasally.

7. The method of claim 5, wherein the composition is administered subcutaneously.

8. The method of claim 5, wherein the composition additionally comprises an adjuvant.


SEQUENCE LISTING (208 sequences)

SEQ ID NO: 1 - protein, 25 aa, Mycobacterium vaccae
  UNSURE (7)...(7)
APVGPGXAAYVQQVPDGPGSVQGMA

SEQ ID NO: 2 - protein, 10 aa, Mycobacterium vaccae
  UNSURE (2)...(2)
MXDQLKVNDD

SEQ ID NO: 3 - protein, 11 aa, Mycobacterium vaccae
  UNSURE (2)...(2)
MXPVPVATAAY

SEQ ID NO: 4 - protein, 21 aa, Mycobacterium vaccae
TPAPAPPPYVDHVEQAKFGDL

SEQ ID NO: 5 - protein, 29 aa, Mycobacterium vaccae
  UNSURE (25)...(25)
MQAFNADAYAFAKREKVSLAPGVPXVFET

SEQ ID NO: 6 - protein, 21 aa, Mycobacterium vaccae
  UNSURE (6)...(6)
MADPNXAILQVSKTTRGGQAA

SEQ ID NO: 7 - protein, 11 aa, Mycobacterium vaccae
MPILQVSQTGR

SEQ ID NO: 8 - protein, 14 aa, Mycobacterium vaccae
  UNSURE (2)...(2)
  UNSURE (6)...(6)
MXDPIXLQLQVSST

SEQ ID NO: 9 - protein, 16 aa, Mycobacterium vaccae
KATYVQGGLGRIEARV

SEQ ID NO: 10 - protein, 9 aa, Mycobacterium vaccae
  UNSURE (2)...(2)
KXGLADLAP

SEQ ID NO: 11 - protein, 14 aa, Mycobacterium vaccae
  UNSURE (12)...(12): Residue can be either Glu or Ile
  UNSURE (2)...(2)
KXYALALMSAVXAA

SEQ ID NO: 12 - protein, 11 aa, Mycobacterium vaccae
  UNSURE (10)...(10)
KNPQVSDELXT

SEQ ID NO: 13 - protein, 21 aa, Mycobacterium vaccae
  UNSURE (9)...(9)
APAPAAPAXGDPAAVVAAMST

SEQ ID NO: 14 - protein, 15 aa, Mycobacterium vaccae
  UNSURE (5)...(5)
EAEVXYLGQPGELVN

SEQ ID NO: 15 - protein, 15 aa, Mycobacterium vaccae
  UNSURE (2)...(2): Residue can be either Gly or Ala
  UNSURE (15)...(15): Residue can be either Pro or Ala
  UNSURE (7)...(7)
AXVVPPXGPPAPGAX

SEQ ID NO: 16 - protein, 15 aa, Mycobacterium vaccae
APAPDLQGPLVSTLS

SEQ ID NO: 17 - protein, 25 aa, Mycobacterium vaccae
ATPDWSGRYTVVTFASDKLGTSVAA

SEQ ID NO: 18 - protein, 25 aa, Mycobacterium vaccae
  UNSURE (15)...(15): Residue can be either Ala or Arg
  UNSURE (23)...(23): Residue can be either Val or Leu
  UNSURE (16)...(16)
APPYDDRGYVDSTAXXASPPTLXVV

SEQ ID NO: 19 - protein, 8 aa, Mycobacterium vaccae
EPEGVAPP

SEQ ID NO: 20 - protein, 25 aa, Mycobacterium vaccae
  UNSURE (21)...(22)
EPAGIPAGFPDVSAYAAVDPXXYVV

SEQ ID NO: 21 - protein, 15 aa, Mycobacterium vaccae
  UNSURE (7)...(7)
APVGPGXAAYVQQVP

SEQ ID NO: 22 - protein, 15 aa, Mycobacterium vaccae
FSRPGLPVEYLMVPS

SEQ ID NO: 23 - protein, 19 aa, Mycobacterium vaccae
FSRPGLPVEYLMVPSPSMG

SEQ ID NO: 24 - protein, 15 aa, Mycobacterium vaccae
FSRPGLPVEYLDVFS

SEQ ID NO: 25 - protein, 14 aa, Mycobacterium vaccae
  UNSURE (1)...(2)
XXTGLHRLRMMVPN

SEQ ID NO: 26 - protein, 20 aa, Mycobacterium vaccae
  UNSURE (16)...(16): Residue can be either Ser or Val
  UNSURE (17)...(17): Residue can be either Gln or Val
VPADPVGAAAQAEPAXXRID

SEQ ID NO: 27 - protein, 14 aa, Mycobacterium vaccae
  UNSURE (4)...(4): Residue can be either Tyr or Pro
  UNSURE (8)...(8): Residue can be either Val or Gly
  UNSURE (9)...(9): Residue can be either Ile or Tyr
  UNSURE (3)...(3)
DPXXDIEXXFARGT

SEQ ID NO: 28 - protein, 15 aa, Mycobacterium vaccae
APSLSVSDYARDAGF

SEQ ID NO: 29 - protein, 16 aa, Mycobacterium vaccae
  UNSURE (2)...(2): Residue can be either Leu or Pro
  UNSURE (1)...(1)
  UNSURE (5)...(5)
  UNSURE (7)...(7)
  UNSURE (10)...(10)
XXLAXAXLGXTVDADQ

SEQ ID NO: 30 - protein, 330 aa, Mycobacterium leprae
MKFVDRFRGAVAGMLRRLVVEAMGVALLSALIGVVGSAPAEAFSRPGLPVEYLQVPSPSMGRDIKVQFQNGGANSPALYLLDGLRAQDDFSGWDINTTAFEWYYQSGISVVMPVGGQSSFYSDWYSPACGKAGCQTYKWETFLTSELPEYLQSNKQIKPTGSAAVGLSMAGLSALTLAIYHPDQFIYVGSMSGLLDPSNAMGPSLIGLAMGDAGGYKAADMWGPSTDPAWKRNDPTVNVGTLIANNTRIWMYCGNGKPTELGGNNLPAKLLEGLVRTSNIKFQDGYNAGGGHNAVFNFPDSGTHSWEYWGEQLNDMKPDLQQYLGATPGA

SEQ ID NO: 31 - protein, 327 aa, Mycobacterium leprae
MIDVSGKIRAWGRWLLVGAAATLPSLISLAGGAATASAFSRPGLPVEYLQVPSEAMGRTIKVQFQNGGNGSPAVYLLDGLRAQDDYNGWDINTSAFEWYYQSGLSVVMPVGGQSSFYSDWYSPACGKAGCTTYKWETFLTSELPKWLSANRSVKSTGSAVVGLSMAGSSALILAAYHPDQFIYAGSLSALMDSSQGIEPQLIGLAMGDAGGYKAADMWGPPNDPAWQRNDPILQAGKLVANNTHLWVYCGNGTPSELGGTNVPAEFLENFVHGSNLKFQDAYNGAGGHNAVFNLNADGTHSWEYWGAQLNAMKPDLQNTLMAVPRSG

SEQ ID NO: 32 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 33 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 34 - protein, 338 aa, Mycobacterium bovis; identical to SEQ ID NO: 32

SEQ ID NO: 35 - protein, 323 aa, Mycobacterium bovis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETLLTSELPQWLSANRAVKPTGSAAIGLSMAGSSAMILAAYHPQQFIYAGSLSALLDPSQGMGLIGLAMGDAGGYKAADMWGPSSDPAWERNDPTQQIPKLVANNTRLWVYCGNGTPNELGGANIPAEFLENFVRSSNLKFQDAYKPAGGHNAVFNFPPNGTHSWEYWGAQLNAMKGDLQSSLGAG

SEQ ID NO: 36 - protein, 333 aa, Mycobacterium leprae
MKFLQQMRKLFGLAAKFPARLTIAVIGTALLAGLVGVVGDTAIAVAFSKPGLPVEYLQVPSPSMGHDIKIQFQGGGQHAVYLLDGLRAQEDYNGWDINTPAFEEYYHSGLSVIMPVGGQSSFYSNWYQPSQGNGQHYTYKWETFLTQEMPSWLQANKNVLPTGNAAVGLSMSGSSALILASYYPQQFPYAASLSGFLNPSEGWWPTMIGLAMNDSGGYNANSMWGPSTDPAWKRNDPMVQIPRLVANNTRIWVYCGNGAPNELGGDNIPAKFLESLTLSTNEIFQNTYAASGGRNGVFNFPPNGTHSWPYWNQQLVAMKPDIQQILNGSNNNA

SEQ ID NO: 37 - protein, 340 aa, Mycobacterium tuberculosis
MTFFEQVRRLRSAATTLPRRVAIAAMGAVLVYGLVGTFGGPATAGAFSRPGLPVEYLQVPSASMGRDIKVQFQGGGPHAVYLLDGLRAQDDYNGWDINTPAFEEYYQSGLSVIMPVGGQSSFYTDWYQPSQSNGQNYTYKWETFLTREMPAWLQANKGVSPTGNAAVGLSMSGGSALILAAYYPQQFPYAASLSGFLNPSEGWWPTLIGLAMNDSGGYNANSMWGPSSDPAWKRNDPMVQIPRLVANNTRIWVYCGNGTPSDLGGDNIPAKFLEGLTLRTNQTFRDTYAADGGRNGVFNFPPNGTHSWPYWNEQLVAMKADIQHVLNGATPPAAPAAPAA

SEQ ID NO: 38 - DNA, 20 bp, Artificial Sequence
  Probe made in a lab
agcggctgggacatcaacac

SEQ ID NO: 39 - DNA, 20 bp, Artificial Sequence
  Probe made in a lab
cagacgcgggtgttgttggc

SEQ ID NO: 40 - DNA, 1211 bp, Mycobacterium vaccae
ggtaccggaagctggaggattgacggtatgagacttcttgacaggattcgtgggccttgggcacgccgtttcggcgtcgtggctgtcgcgacagcgatgatgcctgctttggtgggcctggctggagggtcggcgaccgccggagcattctcccggccaggtctgccggtggagtacctgatggtgccttcgccgtcgatggggcgcgacatcaagatccagttccagagcggtggcgagaactcgccggctctctacctgctcgacggcctgcgtgcgcaggaggacttcaacggctgggacatcaacactcaggctttcgagtggttcctcgacagcggcatctccgtggtgatgccggtcggtggccagtccagcttctacaccgactggtacgcccccgcccgtaacaagggcccgaccgtgacctacaagtgggagaccttcctgacccaggagctcccgggctggctgcaggccaaccgcgcggtcaagccgaccggcagcggccctgtcggtctgtcgatggcgggttcggccgcgctgaacctggcgacctggcacccggagcagttcatctacgcgggctcgatgtccggcttcctgaacccctccgagggctggtggccgttcctgatcaacatctcgatgggtgacgccggcggcttcaaggccgacgacatgtggggcaagaccgaggggatcccaacagcggttggacagcgcaacgatccgatgctgaacatcccgaccctggtcgccaacaacacccgtatctgggtctactgcggtaacggccagcccaccgagctcggcggcggcgacctgcccgccacgttcctcgaaggtctgaccatccgcaccaacgagaccttccgcgacaactacatcgccgcgggtggccacaacggtgtgttcaacttcccggccaacggcacgcacaactgggcgtactggggtcgcgagctgcaggcgatgaagcctgacctgcaggcgcaccttctctgacggttgcacgaaacgaagcccccggccgattgcggccgagggtttcgtcgtccggggctactgtggccgacataaccgaaatcaacgcgatggtggctcatcaggaacgccgagggggtcattgcgctacgacacgaggtgggcgagcaatccttcctgcccgacggagaggtcaacatccacgtcgagtactccagcgtgaa

SEQ ID NO: 41 - DNA, 485 bp, Mycobacterium vaccae
agcggctgggacatcaacaccgccgccttcgagtggtacgtcgactcgggtctcgcggtgatcatgcccgtcggcgggcagtccagcttctacagcgactggtacagcccggcctgcggtaaggccggctgccagacctacaagtgggagacgttcctgacccaggagctgccggcctacctcgccgccaacaagggggtcgacccgaaccgcaacgcggccgtcggtctgtccatggccggttcggcggcgctgacgctggcgatctaccacccgcagcagttccagtacgccgggtcgctgtcgggctacctgaacccgtccgaggggtggtggccgatgctgatcaacatctcgatgggtgacgcgggcggctacaaggccaacgacatgtggggtccaccgaaggacccgagcagcgcctggaagcgcaacgacccgatggtcaacatcggcaagctggtggccaacaacacccccctctc

SEQ ID NO: 42 - DNA, 1052 bp, Mycobacterium vaccae
gttgatgagaaaggtgggttgtttgccgttatgaagttcacagagaagtggcggggctccgcaaaggcggcgatgcaccgggtgggcgttgccgatatggccgccgttgcgctgcccggactgatcggcttcgccgggggttcggcaacggccggggcattctcccggcccggtcttcctgtcgagtacctcgacgtgttctcgccgtcgatgggccgcgacatccgggtccagttccagggtggcggtactcatgcggtctacctgctcgacggtctgcgtgcccaggacgactacaacggctgggacatcaacacccctgcgttcgagtggttctacgagtccggcttgtcgacgatcatgccggtcggcggacagtccagcttctacagcgactggtaccagccgtctcggggcaacgggcagaactacacctacaagtgggagacgttcctgacccaggagctgccgacgtggctggaggccaaccgcggagtgtcgcgcaccggcaacgcgttcgtcggcctgtcgatggcgggcagcgcggcgctgacctacgcgatccatcacccgcagcagttcatctacgcctcgtcgctgtcaggcttcctgaacccgtccgagggctggtggccgatgctgatcgggctggcgatgaacgacgcaggcggcttcaacgccgagagcatgtggggcccgtcctcggacccggcgtggaagcgcaacgacccgatggtcaacatcaaccagctggtggccaacaacacccggatctggatctactgcggcaccggcaccccgtcggagctggacaccgggaccccgggccagaacctgatggccgcgcagttcctcgaaggattcacgttgcggaccaacatcgccttccgtgacaactacatcgcagccggcggcaccaacggtgtcttcaacttcccggcctcgggcacccacagctgggggtactgggggcagcagctgcagcagatgaagcccgacatccagcgggttctgggagctcaggccaccgcctagccacccaccccacaccc

SEQ ID NO: 43 - protein, 326 aa, Mycobacterium vaccae
MRLLDRIRGPWARRFGVVAVATAMMPALVGLAGGSATAGAFSRPGLPVEYLMVPSPSMGRDIKIQFQSGGENSPALYLLDGLRAQEDFNGWDINTQAFEWFLDSGISVVMPVGGQSSFYTDWYAPARNKGPTVTYKWETFLTQELPGWLQANRAVKPTGSGPVGLSMAGSAALNLATWHPEQFIYAGSMSGFLNPSEGWWPFLINISMGDAGGFKADDMWGKTEGIPTAVGQRNDPMLNIPTLVANNTRIWVYCGNGQPTELGGGDLPATFLEGLTIRTNETFRDNYIAAGGHNGVFNFPANGTHNWAYWGRELQAMKPDLQAHLL

SEQ ID NO: 44 - protein, 161 aa, Mycobacterium vaccae
SGWDINTAAFEWYVDSGLAVIMPVGGQSSFYSDWYSPACGKAGCQTYKWETFLTQELPAYLAANKGVDPNRNAAVGLSMAGSAALTLAIYHPQQFQYAGSLSGYLNPSEGWWPMLINISMGDAGGYKANDMWGPPKDPSSAWKRNDPMVNIGKLVANNTPL

SEQ ID NO: 45 - protein, 334 aa, Mycobacterium vaccae
MKFTEKWRGSAKAAMHRVGVADMAAVALPGLIGFAGGSATAGAFSRPGLPVEYLDVFSPSMGRDIRVQFQGGGTHAVYLLDGLRAQDDYNGWDINTPAFEWFYESGLSTIMPVGGQSSFYSDWYQPSRGNGQNYTYKWETFLTQELPTWLEANRGVSRTGNAFVGLSMAGSAALTYAIHHPQQFIYASSLSGFLNPSEGWWPMLIGLAMNDAGGFNAESMWGPSSDPAWKRNDPMVNINQLVANNTRIWIYCGTGTPSELDTGTPGQNLMAAQFLEGFTLRTNIAFRDNYIAAGGTNGVFNFPASGTHSWGYWGQQLQQMKPDIQRVLGAQATA

SEQ ID NO: 46 - DNA, 795 bp, Mycobacterium vaccae
ctgccgcgggtttgccatctcttgggtcctgggtcgggaggccatgttctgggtaacgatccggtaccgtccggcgatgtgaccaacatgcgaacagcgacaacgaagctaggagcggcgctcggcgcagcagcattggtggccgccacggggatggtcagcgcggcgacggcgaacgcccaggaagggcaccaggtccgttacacgctcacctcggccggcgcttacgagttcgacctgttctatctgacgacgcagccgccgagcatgcaggcgttcaacgccgacgcgtatgcgttcgccaagcgggagaaggtcagcctcgccccgggtgtgccgtgggtcttcgaaaccacgatggccgacccgaactgggcgatccttcaggtcagcagcaccacccgcggtgggcaggccgccccgaacgcgcactgcgacatcgccgtcgatggccaggaggtgctcagccagcacgacgacccctacaacgtgcggtgccagctcggtcagtggtgagtcacctcgccgagagtccggccagcgccggcggcagcggctcgcggtgcagcaccccgaggcgctgggtcgcgcgggtcagcgcgacgtaaagatcgctggccccgcgcggcccctcggcgaggatctgctccgggtagaccaccagcacggcgtctaactccagacccttggtctgcgtgggtgccaccgcgcccgggacaccgggcgggccgatcaccacgctggtgccctcccggtccgcctccgcacgcacgaaatcgtcgatggcaccggcga

SEQ ID NO: 47 - protein, 142 aa, Mycobacterium vaccae
MRTATTKLGAALGAAALVAATGMVSAATANAQEGHQVRYTLTSAGAYEFDLFYLTTQPPSMQAFNADAYAFAKREKVSLAPGVPWVFETTMADPNWAILQVSSTTRGGQAAPNAHCDIAVDGQEVLSQHDDPYNVRCQLGQW

SEQ ID NO: 48 - DNA, 300 bp, Mycobacterium vaccae
gccagtgcgccaacggttttcatcgatgccgcacacaaccccggtgggccctgcgcttgccgaaggctgcgcgacgagttcgacttccggtatctcgtcggcgtcgtctcggtgatgggggacaaggacgtggacgggatccgccaggacccgggcgtgccggacgggcgcggtctcgcactgttcgtctcgggcgacaaccttcgaaagggtgcggcgctcaacacgatccagatcgccgagctgctggccgcccagttgtaagtgttccgccgaaattgcattccacgccgataatcg

SEQ ID NO: 49 - DNA, 563 bp, Mycobacterium vaccae
ggatcctcggccggctcaagagtccgcgccgaggtggatgtgacgctggacggctacgagttcagtcgggcctgcgaggcgctgtaccacttcgcctgggacgagttctgcgactggtatgtcgagcttgccaaagtgcaactgggtgaaggtttctcgcacaccacggccgtgttggccaccgtgctcgatgtgctgctcaagcttctgcacccggtcatgccgttcgtcaccgaggtgctgtggaaggccctgaccgggcgggccggcgcgagcgaacgtctgggaaatgtggagtcactggtcgtcgcggactggcccacgcccaccggatacgcgctggatcaggctgccgcacaacggatcgccgacacccagaagttgatcaccgaggtgcgccggttccgcagcgatcagggtctggccgaccgccagcgggtgcctgcccggttgtccggcatcgacaccgcgggtctggacgcccatgtcccggcggtgcgcgcgctggcctggcttgaccgagggtgatgagggcttcaccgcgtccgaatcggtcgaggtgc

SEQ ID NO: 50 - DNA, 434 bp, Mycobacterium vaccae
gggccgggcccgaggatgagcaagttcgaagtcgtcaccgggatggcgttcgcggctttcgccgacgcgcccatcgacgtcgccgtcgtcgaggtcgggctcggtggtcgctgggacgcgacgaacgtggtgaacgcaccggtcgcggtcatcaccccgatcggggtggaccacaccgactacctcggtgacacgatcgccgagatcgccggggagaaggccggaaatcatcacccgccagccgacgacctggtgccgaccgacaccgtcgccgtgctggcgcggcaggttcccgaggccatggaggtgctgctggcccaggcggtgcgctcggatgcggctgtagcgcgcgaggattcggagtgcgcggtgctgggccgtcaggtcgccatcggcggcagctgctccggttgcaggggctcggtggcgtctac

SEQ ID NO: 51 - DNA, 438 bp, Mycobacterium vaccae
ggatcccactcccgcgccggcggcggccagctggtacggccattccagcgtgctgatcgaggtcgacggctaccgcgtgctggccgacccggtgtggagcaacagatgttcgccctcacgggcggtcggaccgcagcgcatgcacgacgtcccggtgccgctggaggcgcttcccgccgtggacgcggtggtgatcgccaacgaccactacgaccacctcgacatcgacaccatcgtcgcgttggcgcacacccagcgggccccgttcgtggtgccgttgggcatcggcgcacacctgcgcaagtggggcgtccccgaggcgcggatcgtcgagttggactggcacgaagcccaccgcatcgacgacctgacgctggtctgcacccccgcccggcacttctccggccggttgttctcccgcgactcgacgctgtgggc

SEQ ID NO: 52 - protein, 87 aa, Mycobacterium vaccae
ASAPTVFIDAAHNPGGPCACRRLRDEFDFRYLVGVVSVMGDKDVDGIRQDPGVPDGRGLALFVSGDNLRKGAALNTIQIAELLAAQL

SEQ ID NO: 53 - protein, 175 aa, Mycobacterium vaccae
GSSAGSRVRAEVDVTLDGYEFSRACEALYHFAWDEFCDWYVELAKVQLGEGFSHTTAVLATVLDVLLKLLHPVMPFVTEVLWKALTGRAGASERLGNVESLVVADWPTPTGYALDQAAAQRIADTQKLITEVRRFRSDQGLADRQRVPARLSGIDTAGLDAHVPAVRALAWLDRG

SEQ ID NO: 54 - protein, 144 aa, Mycobacterium vaccae
GPGPRNSKFEVVTGMAFAAFADAPIDVAVVEVGLGGRWDATNVVNAPVAVITPIGVDHTDYLGDTIAEIAGEKAGNHHPPADDLVPTDTVAVLARQVPEANEVLLAQAVRSDAAVAREDSECAVLGRQVAIGGSCSGCRGSVAS

SEQ ID NO: 55 - protein, 145 aa, Mycobacterium vaccae
DPTPAPAAASWYGHSSVLIEVDGYRVLADPVWSNRCSPSRAVGPQRMHDVPVPLEALPAVDAVVISNDHYDHLDIDTIVALAHTQRAPFVVPLGIGAHLRKWGVPEARIVELDWHEAHRIDDLTLVCTPARHFSGRLFSRDSTLW

SEQ ID NO: 56 - protein, 10 aa, Mycobacterium vaccae
  UNSURE (1)...(1): Residue can be either Gly, Ile, Leu or Val
  UNSURE (2)...(2): Residue can be either Ile, Leu, Gly, or Ala
  UNSURE (5)...(5)
  UNSURE (9)...(9)
XXAPXGDAXR

SEQ ID NO: 57 - protein, 8 aa, Mycobacterium vaccae
  UNSURE (7)...(7): Residue can be either Ile or Leu
PEAEANXR

SEQ ID NO: 58 - protein, 11 aa, Mycobacterium vaccae
  UNSURE (4)...(4): Residue can be either Gln or Gly
  UNSURE (5)...(5): Residue can be either Gly or Gln
TANXXEYYDNR

SEQ ID NO: 59 - protein, 34 aa, Mycobacterium vaccae
NSPRAEAEANLRGYFTANPAEYYDLRGILAPIGD

SEQ ID NO: 60 - DNA, 20 bp, Artificial Sequence
  Made in a lab
ccggtgggcccgggctgcgc

SEQ ID NO: 61 - DNA, 20 bp, Artificial Sequence
  Made in a lab
tggccggccaccacgtggta

SEQ ID NO: 62 - DNA, 313 bp, Mycobacterium vaccae
gccggtgggcccgggctgcgcggaatacgcggcagccaatcccactgggccggcctcggtgcagggaatgtcgcaggacccggtcgcggtggcggcctcgaacaatccggagttgacaacgctgtacggctgcactgtcgggccagctcaatccgcaagtaaacctggtggacaccctcaacagcggtcagtacacggtgttcgcaccgaccaacgcggcatttagcaagctgccggcatccacgatcgacgagctcaagaccaattcgtcactgctgaccagcatcctgacctaccacgtggtggccggcca

SEQ ID NO: 63 - protein, 18 aa, Mycobacterium vaccae
  UNSURE (7)...(17)
EPAGPLPXYNERLHTLXQ

SEQ ID NO: 64 - protein, 25 aa, Mycobacterium vaccae
  UNSURE (21)...(21)
GLDNELSLVDGQGRTLTVQQXDTFL

SEQ ID NO: 65 - protein, 26 aa, Mycobacterium vaccae
  UNSURE (3)...(3)
  UNSURE (21)...(22)
  UNSURE (24)...(24)
DPXPDIEVEFARGTGAEPGLXXVXDA

SEQ ID NO: 66 - DNA, 32 bp, Artificial Sequence
  Made in a lab
accgccctcgagttctcccggccaggtctgcc

SEQ ID NO: 67 - DNA, 32 bp, Artificial Sequence
  Made in a lab
aagcacgagctcagtctcttccacgcggacgt

SEQ ID NO: 68 - DNA, 30 bp, Artificial Sequence
  Made in a lab
catggatccattctcccggcccggtcttcc

SEQ ID NO: 69 - DNA, 26 bp, Artificial Sequence
  Made in a lab
tttgaattctaggcggtggcctgagc

SEQ ID NO: 70 - protein, 161 aa, Mycobacterium vaccae
SGWDINTAAFEWYVDSGLAVIMPVGGQSSFYSDWYSPACGKAGCQTYKWETFLTQELPAYLAANKGVDPNRNAAVGLSMAGSAALTLAIYHPQQFQYAGSLSGYLNPSEGWWPMLINISMGDAGGYKANDMWGRTEDPSSAWKRNDPMVNIGKLVANNTPL

SEQ ID NO: 71 - DNA, 33 bp, Artificial Sequence
  Made in a lab
gagagactcgagaacgcccaggaagggcaccag

SEQ ID NO: 72 - DNA, 32 bp, Artificial Sequence
  Made in a lab
gagagactcgagtgactcaccactgaccgagc

SEQ ID NO: 73 - DNA, 20 bp, Artificial Sequence
  Made in a lab
  unsure (3)...(3)
  unsure (6)...(6)
  unsure (9)...(9)
  unsure (15)...(15)
ggngcngcncargcngarcc

SEQ ID NO: 74 - DNA, 825 bp, Mycobacterium vaccae
ttggatcccactcccgcgccggcggcggccagctggtacggccattccagcgtgctgatcgaggtcgacggctaccgcgtgctggccgacccggtgtggagcaacagatgttcgccctcacgggcggtcggaccgcagcgcatgcacgacgtcccggtgccgctggaggcgcttcccgccgtggacgcggtggtgatcagccacgaccactacgaccacctcgacatcgacaccatcgtcgcgttggcgcacacccagcgggccccgttcgtggtgccgttgggcatcggcgcacacctgcgcaagtggggcgtccccgaggcgcggatcgtcgagttggactggcacgaagcccaccgcatagacgacctgacgctggtctgcacccccgcccggcacttctccggacggttgttctcccgcgactcgacgctgtgggcgtcgtgggtggtcaccggctcgtcgcacaaggcgttcttcggtggcgacaccggatacacgaagagcttcgccgagatcggcgacgagtacggtccgttcgatctgaccctgctgccgatcggggcctaccatcccgcgttcgccgacatccacatgaaccccgaggaggcggtgcgcgcccatctggacctgaccgaggtggacaacagcctgatggtgcccatccactgggcgacattccgcctcgccccgcatccgtggtccgagcccgccgaacgcctgctgaccgctgccgacgccgagcgggtacgcctgaccgtgccgattcccggtcagcgggtggacccggagtcgacgttcgacccgtggtggcggttctgaacc

SEQ ID NO: 75 - protein, 273 aa, Mycobacterium vaccae
LDPTPAPAAASWYGHSSVLIEVDGYRVLADPVWSNRCSPSRAVGPQRMHDVPVPLEALPAVDAVVISHDHYDHLDIDTIVALAHTQRAPFVVPLGIGAHLRKWGVPEARIVELDWHEAHRIDDLTLVCTPARHFSGRLFSRDSTLWASWVVTGSSHKAFFGGDTGYTKSFAEIGDEYGPFDLTLLPIGAYHPAFADIHMNPEEAVRAHLDLTEVDNSLMVPIHWATFRLAPHPWSEPAERLLTAADAERVRLTVPIPGQRVDPESTFDPWWRF

SEQ ID NO: 76 - protein, 10 aa, Mycobacterium vaccae
AKTIAYDEEA

SEQ ID NO: 77 - DNA, 337 bp, Mycobacterium vaccae
gatccctacatcctgctggtcagctccaaggtgtcgaccgtcaaggatctgctcccgctgctggagaaggtcatccaggccggcaagccgctgctgatcatcgccgaggacgtcgagggcgaggccctgtccacgctggtggtcaacaagatccgcggcaccttcaagtccgtcgccgtcaaggctccgggcttcggtgaccgccgcaaggcgatgctgcaggacatggccatcctcaccggtggtcaggtcgtcagcgaaagagtcgggctgtccctggagaccgccgacgtctcgctgctgggccaggcccgcaaggtcgtcgtcaccaaggaca

SEQ ID NO: 78 - protein, 112 aa, Mycobacterium vaccae
DPYILLVSSKVSTVKDLLPLLEKVIQAGKPLLIIAEDVEGEALSTLVVNKIRGTFKSVAVKAPGFGDRRKAMLQDMAILTGGQVVSERVGLSLETADVSLLGQARKVVVTKD

SEQ ID NO: 79 - DNA, 360 bp, Mycobacterium vaccae
ccgtacgagaagatcggcgctgagctggtcaaagaggtcgccaagaagaccgacgacgtcgcgggcgacggcaccaccaccgccaccgtgctcgctcaggctctggttcgcgaaggcctgcgcaacgtcgcagccggcgccaacccgctcggcctcaagcgtggcatcgagaaggctgtcgaggctgtcacccagtcgctgctgaagtcggccaaggaggtcgagaccaaggagcagatttctgccaccgcggcgatctccgccggcgacacccagatcggcgagctcatcgccgaggccatggacaaggtcggcaacgagggtgtcatcaccgtcgaggagtcgaacaccttcggcctg

SEQ ID NO: 80 - protein, 120 aa, Mycobacterium vaccae
PYEKIGAELVKEVAKKTDDVAGDGTTTATVLAQALVREGLRNVAAGANPLGLKRGIEKAVEAVTQSLLKSAKEVETKEQISATAAISAGDTQIGELIAEAMDKVGNEGVITVEESNTFGL

SEQ ID NO: 81 - DNA, 43 bp, Artificial Sequence
  Made in a lab
actgacgctgaggagcgaaagcgtggggagcgaacaggattag

SEQ ID NO: 82 - DNA, 43 bp, Artificial Sequence
  Made in a lab
cgacaaggaacttcgctaccttaggaccgtcatagttacgggc

SEQ ID NO: 83 - DNA, 20 bp, Artificial Sequence
  Made in a lab
aaaaaaaaaaaaaaaaaaaa

SEQ ID NO: 84 - DNA, 31 bp, Artificial Sequence
  Made in a lab
ggaaggaagcggccgcttttttttttttttt

SEQ ID NO: 85 - DNA, 31 bp, Artificial Sequence
  Made in a lab
gagagagagcccgggcatgctsctsctscts

SEQ ID NO: 86 - DNA, 238 bp, Mycobacterium vaccae
ctcgatgaaccgctcggagcgctcgacctgaagctgcgccacgtcatgcagttcgagctcaagcgcatccagcgggaggtcgggatcacgttcatctacgtgacccacgaccaggaagaggcgctcacgatgagtgaccgcatcgcggtgatgaacgccggcaacgtcgaacagatcggcagcccgaccgagatctacgaccgtcccgcgacggtgttcgtcgccagcttcatcgaat

SEQ ID NO: 87 - protein, 79 aa, Mycobacterium vaccae
LDEPLGALDLKLRHVMQFELKRIQREVGITFIYVTHDQEEALTMSDRIAVMNAGNVEQIGSPTEIYDRPATVFVASFIE

SEQ ID NO: 88 - DNA, 1518 bp, Mycobacterium vaccae
cactcgccatgggtgttacaataccccaccagttcctcgaagtaaacgaacagaaccgtgacatccagctgagaaaatattcacagcgacgaagcccggccgatgcctgatggggtccggcatcagtacagcgcgctttcctgcgcggattctattgtcgagtccggggtgtgacgaaggaatccattgtcgaaatgtaaattcgttgcggaatcacttgcataggtccgtcagatccgcgaaggtttaccccacagccacgacggctgtccccgaggaggacctgccctgaccggcacacacatcaccgctgcagaacctgcagaacagacggcggattccgcggcaccgcccaagggcgcgccggtgatcgagatcgaccatgtcacgaagcgcttcggcgactacctggccgtcgcggacgcagacttctccatcgcgcccggggagttcttctccatgctcggcccgtccgggtgtgggaagacgaccacgttgcgcatgatcgcgggattcgagaccccgactgaaggggcgatccgcctcgaaggcgccgacgtgtcgaggaccccacccaacaagcgcaacgtcaacacggtgttccagcactacgcgctgttcccgcacatgacggtctgggacaacgtcgcgtacggcccgcgcagcaagaaactcggcaaaggcgaggtccgcaagcgcgtcgacgagctgctggagatcgtccggctgaccgaatttgccgagcgcaggcccgcccagctgtccggcgggcagcagcagcgggtggcgttggcccgggcactggtgaactaccccagcgcgctgctgctcgatgaaccgctcggagcgctcgacctgaagctgcgccacgtcatgcagttcgagctcaagcgcatccagcgggaggtcgggatcacgttcatctacgtgacccacgaccaggaagaggcgctcacgatgagtgaccgcatcgcggtgatgaacgccggcaacgtcgaacagatcggcagcccgaccgagatctacgaccgtcccgcgacggtgttcgtcgccagcttcatcggacaggccaacctctgggcgggccggtgcaccggccgctccaaccgcgattacgtcgagatcgacgttctcggctcgacgctgaaggcacgcccgggcgagaccacgatcgagcccggcgggcacgccaccctgatggtgcgtccggaacgcatccgggtcaccccgggctcccaggacgcgccgaccggtgacgtcgcctgcgtgcgtgccaccgtcaccgacctgaccttccaaggtccggtggtgcggctctcgctggccgctccggacgactcgaccgtgatcgcccacgtcggccccgagcaggatctgccgctgctgcgccccggcgacgacgtgtacgtcagctgggcaccggaagcctccctggtgcttcccggcgacgacatccccaccaccgaggacctcgaagagatgctcgacgactcctgagtcacgcttcccgattgccga

SEQ ID NO: 89 - protein, 376 aa, Mycobacterium vaccae
VIEIDHVTKRFGDYLAVADADFSIAPGEFFSMLGPSGCGKTTTLRMIAGFETPTEGAIRLEGADVSRTPPNKRNVNTVFQHYALFPHMTVWDNVAYGPRSKKLGKGEVRKRVDELLEIVRLTEFAERRPAQLSGGQQQRVALARALVNYPSALLLDEPLGALDLKLRHVMQFELKRIQREVGITFIYVTHDQEEALTMSDRIAVMNAGNVEQIGSPTEIYDRPATVFVASFIGQANLWAGRCTGRSNRDYVEIDVLGSTLKARPGETTIEPGGHATLMVRPERIRVTPGSQDAPTGDVACVRATVTDLTFQGPVVRLSLAAPDDSTVIAHVGPEQDLPLLRPGDDVYVSWAPEASLVLPGDDIPTTEDLEEMLDDS

SEQ ID NO: 90 - DNA, 33 bp, Artificial Sequence
  Made in a lab
gagagactcgaggtgatcgagatcgaccatgtc

SEQ ID NO: 91 - DNA, 31 bp, Artificial Sequence
  Made in a lab
agagactcgagcaatcgggaagcgtgactca

SEQ ID NO: 92 - DNA, 323 bp, Mycobacterium vaccae
gtcgactacaaagaagacttcaacgacaacgagcagtggttcgccaaggtcaaggagccgttgtcgcgcaagcaggacataggcgccgacctggtgatccccaccgagttcatggccgcgcgcgtcaagggcctgggatggctcaatgagatcagcgaagccggcgtgcccaatcgcaagaatctgcgtcaggacctgttggactcgagcatcgacgagggccgcaagttcaccgcgccgtacatgaccggcatggtcggtctcgcctacaacaaggcagccaccggacgcgatatccgcaccatcgacgacctctgggatcc

SEQ ID NO: 93 - DNA, 1341 bp, Mycobacterium vaccae
ccccacccccttccctggagccgacgaaaggcacccgcacatgtcccgtgacatcgatccccacctgctggcccgaatgaccgcacgccgcaccttgcgtcgccgcttcatcggcggtggcgccgcggccgccgcgggcctgaccctcggttcgtcgttcctggcggcgtgcgggtccgacagtgggacctcgagcaccacgtcacaggacagcggccccgccagcggcgccctgcgcgtctccaactggccgctctatatggccgacggtttcatcgcagcgttccagaccgcctcgggcatcacggtcgactacaaagaagacttcaacgacaacgagcagtggttcgccaaggtcaaggagccgttgtcgcgcaagcaggacataggcgccgacctggtgatccccaccgagttcatggccgcgcgcgtcaagggcctgggatggctcaatgagatcagcgaagccggcgtgcccaatcgcaagaatctgcgtcaggacctgttggactcgagcatcgacgagggccgcaagttcaccgcgccgtacatgaccggcatggtcggtctcgcctacaacaaggcagccaccggacgcgatatccgcaccatcgacgacctctgggatcccgcgttcaagggccgcgtcagtctgttctccgacgtccaggacggcctcggcatgatcatgctctcgcagggcaactcgccggagaatccgaccaccgagtccattcagcaggcggtcgatctggtccgcgaacagaacgacagggggtcagatccgtcgcttcaccggcaacgactacgccgacgacctggccgcagaaacatcgccatcgcgcaggcgtactccggtgacgtcgtgcagctgcaggcggacaaccccgatctgcagttcatcgttcccgaatccggcggcgactggttcgtcgacacgatggtgatcccgtacaccacgcagaaccagaaggccgccgaggcgtggatcgactacatctacgaccgagccaactacgccaagctggtcgcgttcacccagttcgtgcccgcactctcggacatgaccgacgaactcgccaaggtcgatcctgcatcggcggagaacccgctgatcaacccgtcggccgaggtgcaggcgaacctgaagtcgtgggcggcactgaccgacgagcagacgcaggagttcaacactgcgtacgccgccgtcaccggcggctgacgcggtggtagtgccgatgcgaggggcataaatggccctgcggacgcgaggagcataaatggccggtgtcgccaccagcagccgtcagcggacaaggtcgctccgtatctgatggtcct

SEQ ID NO: 94 - protein, 393 aa, Mycobacterium vaccae
MSRDIDPHLLARMTARRTLRRRFIGGGAAAAAGLTLGSSFLAACGSDSGTSSTTSQDSGPASGALRVSNWPLYMADGFIAAFQTASGITVDYKEDFNDNEQWFAKVKEPLSRKQDIGADLVIPTEFMAARVKGLGWLNEISEAGVPNRKNLRQDLLDSSIDEGRKFTAPYMTGMVGLAYNKAATGRDIRTIDDLWDPAFKGRVSLFSDVQDGLGMIMLSQGNSPENPTTESIQQAVDLVREQNDRGSDPSLHRQRLRRRPGRRNIAIAQAYSGDVVQLQADNPDLQFIVPESGGDWFVDTMVIPYTTQNQKAAEAWIDYIYDRANYAKLVAFTQFVPALSDMTDELAKVDPASAENPLINPSAEVQANLKSWAALTDEQTQEFNTAYAAVTGG

SEQ ID NO: 95 - DNA, 22 bp, Mycobacterium vaccae
atgtcccgtgacatcgatcccc

SEQ ID NO: 96 - DNA, 21 bp, Mycobacterium vaccae
atcggcactaccaccgcgtca

SEQ ID NO: 97 - DNA, 861 bp, Mycobacterium vaccae
gccggcgctcgcatatctcgcgatcttcttccgtggtgccgttcttctcgctggcacgcacctcgttgtcggagaccggcggctcggtgttcatgccgacgctgacgttcgcctgggacttcggcaactacgtcgacgcgttcacgatgtaccacgagcagatcttccgctcgttcggctacgcgttcgtcgccacggtgctgtgcctgttgctggcgttcccgctggcctacgtcatcgcgttcaaggccggccggttcaagaacctgatcctggggctggtgatcctgccgttcttcgtcacgttcctgatccgcaccattgcgtggaagacgatcctggccgacgaaggctgggtggtcaccgcgctgggcgccatcgggctgctgcctgacgagggccggctgctgtccaccagctgggcggtcatcggcggtctgacctacaactggatcatcttcatgatcctgccgctgtacgtcagcctggagaagatcgacccgcgtctgctggaggcctcccaggacctctactcgtcggcgccgcgcagcttcggcaaggtgatcctgccgatggcgatgcccggggtgctggccgggagcatgctggtgttcatcccggccgtcggcgacttcatcaacgccgactatctcggcagtacccagaccaccatgatcggcaacgtgatccagaagcagttcctggtcgtcaaggactatccggcggcggccgcgctgagtctggggctgatgttgctgatcctgatcggcgtgctcctctacacacgggcgctgggttcggaggatctggtatgaccacccaggcaggcgccgcactggccaccgccgcccagcaggatcc

SEQ ID NO: 98 - protein, 259 aa, Mycobacterium vaccae
VVPFFSLARTSLSETGGSVFMPTLTFAWDFGNYVDAFTMYHEQIFRSFGYAFVATVLCLLLAFPLAYVIAFKAGRFKNLILGLVILPFFVTFLIRTIAWTILADEGWVVTALGAIGLLPDEGRLLSTSWAVIGGLTYNWIIFMILPLYVSLEKIDPRLLEASQDLYSSAPRSFGKVILPMAMPGVLAGSMLVFIPAVGDFINADYLGSTQTTMIGNVIQKQFLVVKDYPAAAALSLGLMLLILIGVLLYTRALGSEDLV

SEQ ID NO: 99 - DNA, 277 bp, Mycobacterium vaccae
gtaatctttgctggagcccgtacgccggtaggcaaactcatgggttcgctcaaggacttcaagggcagcgatctcggtgccgtggcgatcaagggcgccctggagaaagccttccccggcgtcgacgaccctgctcgtctcgtcgagtacgtgatcatgggccaagtgctctccgccggcgccggccagatgcccgcccgccaggccgccgtcgccgccggcatcccgtgggacgtcgcctcgctgacgatcaacaagatgtgcctgtcgggcatcg

SEQ ID NO: 100 - protein, 92 aa, Mycobacterium vaccae
VIFAGARTPVGKLMGSLKDFKGSDLGAVAIKGALEKAFPGVDDPARLVEYVIMGQVLSAGAGQMPARQAAVAAGIPWDVASLTINKMCLSGI

SEQ ID NO: 101 - protein, 12 aa, Mycobacterium vaccae
  UNSURE (1)...(1): Residue can be either Glu or Pro
  UNSURE (2)...(2): Residue can be either Pro or Glu
  UNSURE (7)...(7)
  UNSURE (12)...(12)
XXADRGXSKYRX

SEQ ID NO: 102 - protein, 24 aa, Mycobacterium vaccae
  UNSURE (1)...(1)
XIDESLFDAEEKMEKAVSVARDSA

SEQ ID NO: 103 - protein, 23 aa, Mycobacterium vaccae
  UNSURE (1)...(2)
  UNSURE (15)...(15)
  UNSURE (17)...(17)
XXIAPATSGTLSEFXAXKGVTME

SEQ ID NO: 104 - protein, 15 aa, Mycobacterium vaccae
PNVPDAFAVLADRVG

SEQ ID NO: 105 - protein, 9 aa, Mycobacterium vaccae
  UNSURE (1)...(1)
XIRVGVNGF

SEQ ID NO: 106 - DNA, 485 bp, Mycobacterium vaccae
agcggctgggacatcaacaccgccgccttcgagtggtacgtcgactcgggtctcgcggtgatcatgcccgtcggcgggcagtccagcttctacagcgactggtacagcccggcctgcggtaaggccggctgccagacctacaagtgggagacgttcctgacccaggagctgccggcctacctcgccgccaacaagggggtcgacccgaaccgcaacgcggccgtcggtctgtccatggccggttcggcggcgctgacgctggcgatctaccacccgcagcagttccagtacgccgggtcgctgtcgggctacctgaacccgtccgaggggtggtggccgatgctgatcaacatctcgatgggtgacgcgggcggctacaaggccaacgacatgtggggtcgcaccgaggacccgagcagcgcctggaagcgcaacgacccgatggtcaacatcggcaagctggtcgccaacaacacccccctctc

SEQ ID NO: 107 - DNA, 501 bp, Mycobacterium vaccae
  unsure (441)...(441)
  unsure (450)...(450)
atgccggtgcgacgtgcgcgcagtgcgcttgcgtccgtgaccttcgtcgcggccgcgtgcgtgggcgctgagggcaccgcactggcggcgacgccggactggagcgggcgctacacggtggtgacgttcgcctccgacaaactcggcacgagtgtggccgcccgccagccagaacccgacttcagcggtcagtacaccttcagcacgtcctgtgtgggcacctgcgtggccaccgcgtccgacggcccggcgccgtcgaacccgacgattccgcagcccgcgcgctacacctgggacggcaggcagtgggtgttcaactacaactggcagtgggagtgcttccgcggcgccgacgtcccgcgcgagtacgccgccgcgcgttcgctggtgttctacgccccgaccgccgacgggtcgatgttcggcacctggcgcaccganatcctgganggcctctgcaagggcaccgtgatcatgccggtcgcggcctatccggcgtag

SEQ ID NO: 108 - DNA, 180 bp, Mycobacterium vaccae
atgaaccagccgcggcccgaggccgaggcgaacctgcggggctacttcaccgccaacccggcggagtactacgacctgcggggcatcctcgccccgatcggtgacgcgcagcgcaactgcaacatcaccgtgctgccggtagagctgcagacggcctacgacacgttcatggccggctga

SEQ ID NO: 109 - protein, 166 aa, Mycobacterium vaccae
MPVRRARSALASVTFVAAACVGAEGTALAATPDWSGRYTVVTFASDKLGTSVAARQPEPDFSGQYTFSTSCVGTCVATASDGPAPSNPTIPQPARYTWDGRQWVFNYNWQWECFRGADVPREYAAARSLVFYAPTADGSMFGTWRTDILDGLCKGTVIMPVAAYPA

SEQ ID NO: 110 - protein, 74 aa, Mycobacterium vaccae
PRDTHPGANQAVTAAMNQPRPEAEANLRGYFTANPAEYYDLRGILAPIGDAQRNCNITVLPVELQTAYDTFMAG

SEQ ID NO: 111 - DNA, 503 bp, Mycobacterium vaccae
  unsure (358)...(358)
atgcaggtgcggcgtgttctgggcagtgtcggtgcagcagtcgcggtttcggccgcgttatggcagacgggggtttcgataccgaccgcctcagcggatccgtgtccggacatcgaggtgatcttcgcgcgcgggaccggtgcggaacccggcctcgggtgggtcggtgatgcgttcgtcaacgcgctgcggcccaaggtcggtgagcagtcggtgggcacctacgcggtgaactacccggcaggattcggacttcgacaaatcggcgcccatgggcgcggccgacgcatcggggcgggtgcagtggatggccgacaactgcccggacaccaagcttgtcctgggcggcatgtcgcanggcgccggcgtcatcgacctgatcaccgtcgatccgcgaccgctgggccggttcacccccaccccgatgccgccccgcgtcgccgaccacgtggccgccgttgtggtcttcggaaatccgttgcgcgacatccgtggtggcggtc

SEQ ID NO: 112 - protein, 167 aa, Mycobacterium vaccae
  UNSURE (119)...(119)
MQVRRVLGSVGAAVAVSAALWQTGVSIPTASADPCPDIEVIFARGTGAEPGLGWVGDAFVNALRPKVGEQSVGTYAVNYPAGFDFDKSAPMGAADASGRVQWMADNCPDTKLVLGGMSXGAGVIDLITVDPRPLGRFTPTPMPPRVADHVAAVVVFGNPLRDIRGGG

SEQ ID NO: 113 - DNA, 1569 bp, Mycobacterium vaccae
atggccaagacaattgcgtatgacgaagaggcccgccgtggcctcgagcggggcctcaacgccctcgcagacgccgtaaaggtgacgttgggcccgaagggtcgcaacgtcgtgctggagaagaagtggggcgcccccacgatcaccaacgatggtgtgtccatcgccaaggagatcgagctggaggacccgtacgagaagatcggcgctgagctggtcaaagaggtcgccaagaagaccgacgacgtcgcgggcgacggcaccaccaccgccaccgtgctcgctcaggctctggttcgcgaaggcctgcgcaacgtcgcagccggcgccaacccgctcggcctcaagcgtggcatcgagaaggctgtcgaggctgtcacccagtcgctgctgaagtcggccaaggaggtcgagaccaaggagcagatttctgccaccgcggcgatttccgccggcgacacccagatcggcgagctcatcgccgaggccatggacaaggtcggcaacgagggtgtcatcaccgtcgaggagtcgaacaccttcggcctgcagctcgagctcaccgagggtatgcgcttcgacaagggctacatctcgggttacttcgtgaccgacgccgagcgccaggaagccgtcctggaggatccctacatcctgctggtcagctccaaggtgtcgaccgtcaaggatctgctcccgctgctggagaaggtcatccaggccggcaagccgctgctgatcatcgccgaggacgtcgagggcgaggccctgtccacgctggtggtcaacaagatccgcggcaccttcaagtccgtcgccgtcaaggctccgggcttcggtgaccgccgcaaggcgatgctgcaggacatggccatcctcaccggtggtcaggtcgtcagcgaaagagtcgggctgtccctggagaccgccgacgtctcgctgctgggccaggcccgcaaggtcgtcgtcaccaaggacgagaccaccatcgtcgagggctcgggcgattccgatgccatcgccggccgggtggctcagatccgcgccgagatcgagaacagcgactccgactacgaccgcgagaagctgcaggagcgcctggccaagctggccggcggtgttgcggtgatcaaggccggagctgccaccgaggtggagctcaaggagcgcaagcaccgcatcgaggacgccgtccgcaacgcgaaggctgccgtcgaagagggcatcgtcgccggtggcggcgtggctctgctgcagtcggctcctgcgctggacgacctcggcctgacgggcgacgaggccaccggtgccaacatcgtccgcgtggcgctgtcggctccgctcaagcagatcgccttcaacggcggcctggagcccggcgtcgttgccgagaaggtgtccaacctgcccgcgggtcacggcctcaacgccgcgaccggtgagtacgaggacctgctcaaggccggcgtcgccgacccggtgaaggtcacccgctcggcgctgcagaacgcggcgtccatcgcggctctgttcctcaccaccgaggccgtcgtcgccgacaagccggag

SEQ ID NO: 114 - protein, 523 aa, Mycobacterium vaccae
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEAVTQSLLKSAKEVETKEQISATAAISAGDTQIGELIAEAMDKVGNEGVITVEESNTFGLQLELTEGMRFDKGYISGYFVTDAERQEAVLEDPYILLVSSKVSTVKDLLPLLEKVIQAGKPLLIIAEDVEGEALSTLVVNKIRGTFKSVAVKAPGFGDRRKAMLQDMAILTGGQVVSERVGLSLETADVSLLGQARKVVVTKDETTIVEGSGDSDAIAGRVAQIRAEIENSDSDYDREKLQERLAKLAGGVAVIKAGAATEVELKERKHRIEDAVRNAKAAVEEGIVAGGGVALLQSAPALDDLGLTGDEATGANIVRVALSAPLKQIAFNGGLEPGVVAEKVSNLPAGHGLNAATGEYEDLLKAGVADPVKVTRSALQNAASIAALFLTTEAVVADKPE

SEQ ID NO: 115 - DNA, 647 bp, Mycobacterium vaccae
atggccaagacaattgcgtatgacgaagaggcccgccgtggcctcgagcggggcctcaacgccctcgcagacgccgtaaaggtgacgttgggcccgaagggtcgcaacgtcgtgctggagaagaagtggggcgcccccacgatcaccaacgatggtgtgtccatcgccaaggagatcgagctggaggacccgtacgagaagatcggcgctgagctggtcaaagaggtcgccaagaagaccgacgacgtcgcgggcgacggcaccaccaccgccaccgtgctcgctcaggctctggttcgcgaaggcctgcgcaacgtcgcagccggcgccaacccgctcggcctcaagcgtggcatcgagaaggctgtcgaggctgtcacccagtcgctgctgaagtcggccaaggaggtcgagaccaaggagcagatttctgccaccgcggcgatttccgccggcgacacccagatcggcgagctcatcgccgaggccatggacaaggtcggcaacgagggtgtcatcaccgtcgaggagtcgaacaccttcggcctgcagctcgagctcaccgagggtatgcgcttcgacaagggctacatctcgggttacttcgtgaccgacgccgagcgccaggaagccgtcctggaggatcc

SEQ ID NO: 116 - DNA, 927 bp, Mycobacterium vaccae
gatccctacatcctgctggtcagctccaaggtgtcgaccgtcaaggatctgctcccgctgctggagaaggtcatccaggccggcaagccgctgctgatcatcgccgaggacgtcgagggcgaggccctgtccacgctggtggtcaacaagatccgcggcaccttcaagtccgtcgccgtcaaggctccgggcttcggtgaccgccgcaaggcgatgctgcaggacatggccatcctcaccggtggtcaggtcgtcagcgaaagagtcgggctgtccctggagaccgccgacgtctcgctgctgggccaggcccgcaaggtcgtcgtcaccaaggacgagaccaccatcgtcgagggctcgggcgattccgatgccatcgccggccgggtggctcagatccgcgccgagatcgagaacagcgactccgactacgaccgcgagaagctgcaggagcgcctggccaagctggccggcggtgttgcggtgatcaaggccggagctgccaccgaggtggagctcaaggagcgcaagcaccgcatcgaggacgccgtccgcaacgcgaaggctgccgtcgaagagggcatcgtcgccggtggcggcgtggctctgctgcagtcggctcctgcgctggacgacctcggcctgacgggcgacgaggccaccggtgccaacatcgtccgcgtggcgctgtcggctccgctcaagcagatcgccttcaacggcggcctggagcccggcgtcgttgccgagaaggtgtccaacctgcccgcgggtcacggcctcaacgccgcgaccggtgagtacgaggacctgctcaaggccggcgtcgccgacccggtgaaggtcacccgctcggcgctgcagaacgcggcgtccatcgcggctctgttcctcaccaccgaggccgtcgtcgccgacaagccggag

SEQ ID NO: 117 - protein, 215 aa, Mycobacterium vaccae
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEAVTQSLLKSAKEVETKEQISATAAISAGDTQIGELIAEAMDKVGNEGVITVEESNTFGLQLELTEGMRFDKGYISGYFVTDAERQEAVLED

SEQ ID NO: 118 - protein, 309 aa, Mycobacterium vaccae
DPYILLVSSKVSTVKDLLPLLEKVIQAGKPLLIIAEDVEGEALSTLVVNKIRGTFKSVAVKAPGFGDRRKAMLQDMAILTGGQVVSERVGLSLETADVSLLGQARKVVVTKDETTIVEGSGDSDAIAGRVAQIRAEIENSDSDYDREKLQERLAKLAGGVAVIKAGAATEVELKERKHRIEDAVRNAKAAVEEGIVAGGGVALLQSAPALDDLGLTGDEATGANIVRVALSAPLKQIAFNGGLEPGVVAEKVSNLPAGHGLNAATGEYEDLLKAGVADPVKVTRSALQNAASIAALFLTTEAVVADKPE

SEQ ID NO: 119 - DNA, 162 bp, Mycobacterium vaccae
ctcgtacaggcgacggagatctccgacgacgccacgtcggtacggttggtcgccaccctgttcggcgtcgtgttgttgacgttggtgctgtccgggctcaacgccaccctcatccagggcgcaccagaagacagctggcgcaggcggattccgtcgatcttc

SEQ ID NO: 120 - DNA, 1366 bp, Mycobacterium vaccae
  unsure (955)...(955)
  unsure (973)...(973)
gatgagcagcgtgctgaactcgacctggttggcctgggccgtcgcggtcgcggtcgggttcccggtgctgctggtcgtgctgaccgaggtgcacaacgcgttgcgtcggcgcggcagcgcgctggcccgcccggtgcaactcctgcgtacctacatcctgccgctgggcgcgttgctgctcctgctggtacaggcgatggagatctccgacgacgccacgtcggtacggttggtcgccaccctgttcggcgtcgtgttgttgacgttggtgctgtccgggctcaacgccaccctcatccagggcgcaccagaagacagctggcgcaggcggattccgtcgatcttcctcgacgtcgcgcgcttcgcgctgatcgcggtcggtatcaccgtgatcatggcctatgtctggggcgcgaacgtggggggcctgttcaccgcactgggcgtcacttccatcgttcttggcctggctctgcagaattcggtcggtcagatcatctcgggtctgctgctgctgttcgagcaaccgttccggctcggcgactggatcaccgtccccaccgcggcgggccggccgtccgcccacggccgcgtggtggaagtcaactggcgtgcaacacatatcgacaccggcggcaacctgctggtaatgcccaacgccgaactcgccggcgcgtcgttcaccaattacagccggcccgtgggagagcaccggctgaccgtcgtcaccaccttcaacgccgcggacacccccgatgatgtctgcgagatgctgtcgtcggtcgcggcgtcgctgcccgaactgcgcaccgacggacagatcgccacgctctatctcggtgcggccgaatacgagaagtcgatcccgttgcacacacccgcggtggacgactcggtcaggagcacgtacctgcgatgggtctggtacgccgcgcgccggcaggaacttcgcctnaacggcgtcgccgacganttcgacacgccggaacggatcgcctcggccatgcgggctgtggcgtccacactgcgcttggcagacgacgaacagcaggagatcgccgacgtggtgcgtctggtccgttacggcaacggggaacgcctccagcagccgggtcaggtaccgaccgggatgaggttcatcgtagacggcagggtgagtctgtccgtgatcgatcaggacggcgacgtgatcccggcgcgggtgctcgagcgtggcgacttcctggggcagaccacgctgacgcgggaaccggtactggcgaccgcgcacgcgctggaggaagtcaccgtgctggagatggcccgtgacgagatcgagcgcctggtgcaccgaaagccgatcctgctgcacgtgatcggggccgtg

SEQ ID NO: 121 - protein, 455 aa, Mycobacterium vaccae
  UNSURE (318)...(318)
  UNSURE (324)...(324)
MSSVLNSTWLAWAVAVAVGFPVLLVVLTEVHNALRRRGSALARPVQLLRTYILPLGALLLLLVQAMEISDDATSVRLVATLFGVVLLTLVLSGLNATLIQGAPEDSWRRRIPSIFLDVARFALIAVGITVIMAYVWGANVGGLFTALGVTSIVLGLALQNSVGQIISGLLLLFEQPFRLGDWITVPTAAGRPSAHGRVVEVNWRATHIDTGGNLLVMPNAELAGASFTNYSRPVGEHRLTVVTTFNAADTPDDVCEMLSSVAASLPELRTDGQIATLYLGAAEYEKSIPLHTPAVDDSVRSTYLRWVWYAARRQELRXNGVADXFDTPERIASAMRAVASTLRLADDEQQEIADVVRLVRYGNGERLQQPGQVPTGMRFIVDGRVSLSVIDQDGDVIPARVLERGDFLGQTTLTREPVLATAHALEEVTVLEMARDEIERLVHRKPILLHVIGAV

SEQ ID NO: 122 - DNA, 898 bp, Mycobacterium vaccae
atgacaattctgccctggaatgcgcgaacgtctgaacacccgacgcgaaaaagacgcgggcgctaccacctcctgtcgcggatgagcatccagtccaagttgctgctgatgctgcttctgaccagcattctctcggctgcggtggtcggtttcatcggctatcagtccggacggtcctcgctgcgcgcatcggtgttcgaccgcctcaccgacatccgcgagtcgcagtcgcgcgggttggagaatcagttcgcggacctgaagaactcgatggtgatttactcgcgcggcagcactgccacggaggcgatcggcgcgttcagcgacggtttccgtcagctcggcgatgcgacgatcaataccgggcaggcggcgtcattgcgccgttactacgaccggacgttcgccaacaccaccctcgacgacagcggaaaccgcgtcgacgtccgcgcgctcatcccgaaatccaacccccagcgctatctgcaggcgctctataccccgccgtttcagaactgggagaaggcgatcgcgttcgacgacgcgcgcgacggcagcgcctggtcggccgccaatgccagattcaacgagttcttccgcgagatcgtgcaccgcttcaacttcgaggatctgatgctgctcgacctcgagggcaacgtggtgtactccgcctacaaggggccggatctcgggacaaacatcgtcaacggcccctatcgcaaccgggaactgtcggaagcctacgagaaggcggtcgcgtcgaactcgatcgactatgtcggtgtcaccgacttcgggtggtacctgcctgccgaggaaccgaccgcctggttcctgtccccggtcgggttgaaggaccgagtcgacggtgtgatggcggtccagttccccggaattc

SEQ ID NO: 123 - DNA, 1259 bp, Mycobacterium vaccae
cgcaattgatgacggcgcggggacagtggcgtgacaccgggatgggagacaccggtgagaccatcctggtcggaccggacaatctgatgcgctcggactcccggctgttccgcgagaaccgggagaagttcctggccgacgtcgtcgaggggggaaccccgccggaggtcgccgacgaatcggttgaccgccgcggcaccacgctggtgcagccggtgaccacccgctccgtcgaggaggcccaacgcggcaacaccgggacgacgatcgaggacgactatctcggccacgaggcgttacaggcgtactcaccggtggacctgccgggactgcactgggtgatcgtggccaagatcgacaccgacgaggcgttcgccccggtggcgcagttcaccaggaccctggtgctgtcgacggtgatcatcatcttcggcgtgtcgctggcggccatgctgctggcgcggttgttcgtccgtccgatccggcggttgcaggccggcgcccagcagatcagcggcggtgactaccgcctcgctctgccggtgttgtctcgtgacgaattcggcgatctgacaacagctttcaacgacatgagtcgcaatctgtcgatcaaggacgagctgctcggcgaggagcgcgccgagaaccaacggctgatgctgtccctgatgcccgaaccggtgatgcagcgctacctcgacggggaggagacgatcgcccaggaccacaagaacgtcacggtgatcttcgccgacatgatgggcctcgacgagttgtcgcgcatgttgacctccgaggaactgatggtggtggtcaacgacctgacccgccagttcgacgccgccgccgagagtctcggggtcgaccacgtgcggacgctgcacgacgggtacctggccagctgcgggttaggcgtgccgcggctggacaacgtccggcgcacggtcaatttcgcgatcgaaatggaccgcatcatcgaccggcacgccgccgagtccgggcacgacctgcggctccgcgcgggcatcgacaccgggtcggcggccagcgggctggtggggcggtccacgttggcgtacgacatgtggggttcggcggtcgatgtcgcctaccaggtgcagcgcggctccccccagcccggcatctacgtcacctcgcgggtgcacgaggtcatgcaggaaactctcgacttcgtcgccgccggggaggtcgtcggcgagcgcggcgtcgagacggtctggcggttgcagggccacccg

SEQ ID NO: 124 - protein, 299 aa, Mycobacterium vaccae
MTILPWNARTSEHPTRKRRGRYHLLSRMSIQSKLLLMLLLTSILSAAVVGFIGYQSGRSSLRASVFDRLTDIRESQSRGLENQFADLKNSMVIYSRGSTATEAIGAFSDGFRQLGDATINTGQAASLRRYYDRTFANTTLDDSGNRVDVRALIPKSNPQRYLQALYTPPFQNWEKAIAFDDARDGSAWSAANARFNEFFREIVHRFNFEDLMLLDLEGNVVYSAYKGPDLGTNIVNGPYRNRELSEAYEKAVASNSIDYVGVTDFGWYLPAEEPTAWFLSPVGLKDRVDGVMAVQFPGI

SEQ ID NO: 125 - protein, 419 aa, Mycobacterium vaccae
QLMTARGQWRDTGMGDTGETILVGPDNLMRSDSRLFRENREKFLADVVEGGTPPEVADESVDRRGTTLVQPVTTRSVEEAQRGNTGTTIEDDYLGHEALQAYSPVDLPGLHWVIVAKIDTDEAFAPVAQFTRTLVLSTVIIIFGVSLAAMLLARLFVRPIRRLQAGAQQISGGDYRLALPVLSRDEFGDLTTAFNDMSRNLSIKDELLGEERAENQRLMLSLMPEPVMQRYLDGEETIAQDHKNVTVIFADMMGLDELSRMLTSEELMVVVNDLTRQFDAAAESLGVDHVRTLHDGYLASCGLGVPRLDNVRRTVNFAIEMDRIIDRHAAESGHDLRLRAGIDTGSAASGLVGRSTLAYDMWGSAVDVAYQVQRGSPQPGIYVTSRVHEVMQETLDFVAAGEVVGERGVETVWRLQGHP

SEQ ID NO: 126 - DNA, 27 bp, Artificial Sequence
  Made in a lab
ccggatccgatgagcagcgtgctgaac

SEQ ID NO: 127 - DNA, 26 bp, Artificial Sequence
  Made in a lab
gcggatcccacggccccgatcacgtg

SEQ ID NO: 128 - DNA, 33 bp, Artificial Sequence
  Made in a lab
ccggatccaatgacatttctgccctggaatgcg

SEQ ID NO: 129 - DNA, 32 bp, Artificial Sequence
  Made in a lab
ccggatccattcggtggccctgcaaccgccag

SEQ ID NO: 130 - DNA, 27 bp, Artificial Sequence
  Made in a lab
ccggatccggagcaaccgttccggctc

SEQ ID NO: 131 - DNA, 27 bp, Artificial Sequence
  Made in a lab
ccggatcccggctatcagtccggacgg

SEQ ID NO: 132 - DNA, 844 bp, Mycobacterium vaccae
gagcaaccgttccggctcggcgactggatcaccgtccccaccgcggcgggccggccgtccgcccacggccgcgtggtggaagtcaactggcgtgcaacacatatcgacaccggcggcaacctgctggtaatgcccaacgccgaactcgccggcgcgtcgttcaccaattacagccggcccgtgggagagcaccggctgaccgtcgtcaccaccttcaacgccgcggacacccccgatgatgtctgcgagatgctgtcgtcggtcgcggcgtcgctgcccgaactgcgcaccgacggacagatcgccacgctctatctcggtgcggccgaatacgagaagtcgatcccgttgcacacacccgcggtggacgactcggtcaggagcacgtacctgcgatgggtctggtacgccgcgcgccggcaggaacttcgcctaacggcgtcgccgacgattcgacacgccggaacggatcgcctcggccatgcgggctgtggcgtccacactgcgcttggcagacgacgaacagcaggagatcgccgacgtggtgcgtctggtccgttacggcaacggggaacgcctccagcagccgggtcaggtaccgaccgggatgaggttcatcgtagacggcagggtgagtctgtccgtgatcgatcaggacggcgacgtgatcccggcgcgggtgctcgagcgtggcgacttcctggggcagaccacgctgacgcgggaaccggtactggcgaccgcgcacgcgctggaggaagtcaccgtgctggagatggcccgtgacgagatcgagcgcctggtgcaccgaaagccgatcctgctgcacgtgatcggggccgtg

SEQ ID NO: 133 - DNA, 742 bp, Mycobacterium vaccae
ggctatcagtccggacggtcctcgctgcgcgcatcggtgttcgaccgcctcaccgacatccgcgagtcgcagtcgcgcgggttggagaatcagttcgcggacctgaagaactcgatggtgatttactcgcgcggcagcactgccacggaggcgatcggcgcgttcagcgacggtttccgtcagctcggcgatgcgacgatcaataccgggcaggcggcgtcattgcgccgttactacgaccggacgttcgccaacaccaccctcgacgacagcggaaaccgcgtcgacgtccgcgcgctcatcccgaaatccaacccccagcgctatctgcaggcgctctataccccgccgtttcagaactgggagaaggcgatcgcgttcgacgacgcgcgcgacggcagcgcctggtcggccgccaatgccagattcaacgagttcttccgcgagatcgtgcaccgcttcaacttcgaggatctgatgctgctcgacctcgagggcaacgtggtgtactccgcctacaaggggccggatctcgggacaaacatcgtcaacggcccctatcgcaaccgggaactgtcggaagcctacgagaaggcggtcgcgtcgaactcgatcgactatgtcggtgtcaccgacttcgggtggtacctgcctgccgaggaaccgaccgcctggttcctgtccccggtcgggttgaaggaccgagtcgacggtgtgatggcggtccagttccccggaattc

SEQ ID NO: 134 - protein, 282 aa, Mycobacterium vaccae
  UNSURE (145)...(145)
  UNSURE (151)...(151)
EQPFRLGDWITVPTAAGRPSAHGRVVEVNWRATHIDTGGNLLVMPNAELAGASFTNYSRPVGEHRLTVVTTFNAADTPDDVCEMLSSVAASLPELRTDGQIATLYLGAAEYEKSIPLHTPAVDDSVRSTYLRWVWYAARRQELRXNGVADXFDTPERIASAMRAVASTLRLADDEQQEIADVVRLVRYGNGERLQQPGQVPTGMRFIVDGRVSLSVIDQDGDVIPARVLERGDFLGQTTLTREPVLATAHALEEVTVLEMARDEIERLVHRKPILLHVIGAV

SEQ ID NO: 135 - protein, 247 aa, Mycobacterium vaccae
GYQSGRSSLRASVFDRLTDIRESQSRGLENQFADLKNSMVIYSRGSTATEAIGAFSDGFRQLGDATINTGQAASLRRYYDRTFANTTLDDSGNRVDVRALIPKSNPQRYLQALYTPPFQNWEKAIAFDDARDGSAWSAANARFNEFFREIVHRFNFEDLMLLDLEGNVVYSAYKGPDLGTNIVNGPYRNRELSEAYEKAVASNSIDYVGVTDFGWYLPAEEPTAWFLSPVGLKDRVDGVMAVQFPGI

SEQ ID NO: 136 - DNA, 45 bp, Mycobacterium vaccae
  unsure (18)...(18)
atgagcgaaatcgcccgnccctggcgggttctggcatgtggcatc

SEQ ID NO: 137 - DNA, 340 bp, Mycobacterium vaccae
  unsure (273)...(273)
  unsure (286)...(286)
gccaccggcggcgccgccgcggtgcccgccggggtgagcgccccggcggtcgcgccggcccccgcgatgcccgcccgcccggtgtccacgatcgcgccggcgacctcgggcacgctcagcgagtttttcgccgccaagggcgtcacgatggagccgcagtccagccgcgacttccgcgccctcaacatcgtgctgccgaagccgcggggctgggagcacatcccggacccgaacgtgccggacgcgttcgcggtgctggccgaccgggtcagnggtaaaggtcagnagtcgacaaacgcccacgtggtggtcgacaaacacgtaggcgagttcgacggca

SEQ ID NO: 138 - DNA, 235 bp, Mycobacterium vaccae
  unsure (16)...(16)
ggtgaccaccagcgtngaacaggtcgttgccgaagccgcggaggccaccgacgcgattgtcaacggcttcaaggtcagcgttccgggtccgggtccggccgcaccgccacctgcacccggtgcccccggtgtcccgcccgcccccggcgccccggcgctgccgctggccgtcgcaccacccccggctcccgctgttcccgccgtggcgcccgcgccacagctgctgggactgcag

SEQ ID NO: 139 - protein, 15 aa, Mycobacterium vaccae
MSEIARPWRVLACGI

SEQ ID NO: 140 - protein, 113 aa, Mycobacterium vaccae
  UNSURE (96)...(96)
ATGGAAAVPAGVSAPAVAPAPAMPARPVSTIAPATSGTLSEFFAAKGVTMEPQSSRDFRALNIVLPKPRGWEHIPDPNVPDAFAVLADRVGGKGQXSTNAHVVVDKHVGEFDG

SEQ ID NO: 141 - protein, 73 aa, Mycobacterium vaccae
VTTSVEQVVAAADATEAIVNGFKVSVPGPGPAAPPPAPGAPGVPPAPGAPALPLAVAPPPAPAVPAVAPAPQL

SEQ ID NO: 142 - DNA, 273 bp, Mycobacterium vaccae
gcgacctacgtgcaggggggtctcggccgcatcgaggcccgggtggccgacagcggatacagcaacgccgcggccaagggctacttcccgctgagcttcaccgtcgccggcatcgaccagaacggtccgatcgtgaccgccaacgtcaccgcggcggccccgacgggcgccgtggccacccagccgctgacgttcatcgccgggccgagcccgaccggatggcagctgtccaagcagtccgcactggccctgatgtccgcggtcatcgccgca

SEQ ID NO: 143 - protein, 91 aa, Mycobacterium vaccae
ATYVQGGLGRIEARVADSGYSNAAAKGYFPLSFTVAGIDQNGPIVTANVTAAAPTGAVATQPLTFIAGPSPTGWQLSKQSALALMSAVIAA

SEQ ID NO: 144 - DNA, 554 bp, Mycobacterium vaccae
gatgtcacgcccggagaatgtaacgttcgaccggagaacgccgtcggcacaacgagttacgtttgagcacttcagatctcggttaccttggatttcaggcgggggaagcagtaaccgatccaagattcgaaggacccaaacaacatgaaattcactggaatgaccgtgcgcgcaagccgcgcgccctggccggcgtcggggcggcatgtctgttcggcggcgtggccgcggcaaccgtggcggcacagatggcgggcgcccagccggccgagtgcaacgccagctcactcaccggcaccgtcagctcggtgaccggtcaggcgcgtcagtacctagacacccacccgggcgccaaccaggccgtcaccgcggcgatgaaccagccgcggcccgaggccgaggcgaacctgcggggctacttcaccgccaacccggcggagtactacgacctgcggggcatcctcgccccgatcggtgacgcgcagcgcaactgcaacatcaccgtgctgccggtagagctgcagacggcctacgacacgttcatggccggctga

SEQ ID NO: 145 - protein, 136 aa, Mycobacterium vaccae
MKFTGMTVRASRRALAGVGAACLFGGVAAATVAAQMAGAQPAECNASSLTGTVSSVTGQARQYLDTHPGANQAVTAAMNQPRPEAEANLRGYFTANPAEYYDLRGILAPIGDAQRNCNITVLPVELQTAYDTFMAG

SEQ ID NO: 146 - DNA, 808 bp, Mycobacterium vaccae
  unsure (15)...(15)
ccaagtgtgacgcgngtgtgacggtagacgttccgaccaatccaacgacgccgcagctgggaatcacccgtgtgccaattcagtgcgggcaacggtgtccgtccacgaagggattcaggaaatgatgacaactcgccggaagtcagccgcagtggcgggaatcgctgcggtggccatcctcggtgcggccgcatgttcgagtgaggacggtgggagcacggcctcgtcggccagcagcacggcctcctccgcgatggagtccgcgaccgacgagatgaccacgtcgtcggcggccccttcggccgaccctgcggccaacctgatcggctccggctgcgcggcctacgccgagcaggtccccgaaggtcccgggtcggtggccgggatggcagccgatccggtgacggtggcggcgtcgaacaacccgatgctgcagacgctgtcccaggcgctgtccggccagctcaatccgcaggtcaatctcgtcgacaccctcgacggcggtgagttcaccgtgttcgcgccgaccgacgacgcgttcgccaagatcgatccggccacgctggagaccctcaagacggactccgacatgctgaccaacatcctgacctaccacgtcgtgcccggccaggccgcgcccgatcaggtggtcggcgagcatgtgacggtggagggggcgccggtcacggtgtccgggatggccgaccagctcaaggtcaacgacgcgtcggtggtgtgcggtggggtgcagaccgccaacgcgacggtgtatctgatcgacaccgtgctgatgccgccggcagcgtag

SEQ ID NO: 147 - protein, 228 aa, Mycobacterium vaccae
MMTTRRKSAAVAGIAAVAILGAAACSSEDGGSTASSASSTASSAMESATDEMTTSSAAPSADPAANLIGSGCAAYAEQVPEGPGSVAGMAADPVTVAASNNPMLQTLSQALSGQLNPQVNLVDTLDGGEFTVFAPTDDAFAKIDPATLETLKTDSDMLTNILTYHVVPGQAAPDQVVGEHVTVEGAPVTVSGMADQLKVNDASVVCGGVQTANATVYLIDTVLMPPAA

SEQ ID NO: 148 - DNA, 22 bp, Artificial Sequence
  Made in a lab
  unsure (12)...(12)
  unsure (17)...(17)
gcsccsgtsggnccggntgygc

SEQ ID NO: 149 - DNA, 21 bp, Artificial Sequence
  Made in a lab
  unsure (10)...(10)
  unsure (13)...(13)
  unsure (16)...(16)
  unsure (20)...(20)
rtasgcsgcngtngcnacngg

SEQ ID NO: 150 - DNA, 102 bp, Artificial Sequence
  Made in a lab
gcccccgtcggccccggctgtgcggcctacgtgcaacaggtgccggacgggccgggatcggtgcagggcatggcgagctcgcccgtagcgaccgccgcgtat

SEQ ID NO: 151 - DNA, 683 bp, Mycobacterium vaccae
gcccgccaactaaaaccgccgatcatccactgcaggaaggaatctcacgatcatgaacatcagcatgaaaactcttgccggagcgggtttcgcgatgaccgccgccgtcggtctgtcgctgggtaccgcaggcagcgccgcagccgcgccggtcggaccggggtgtgcggcctacgtgcaacaggtgccggacgggccgggatcggtgcagggcatggcgagctcgccggtggccaccgcggcggccgacaacccgctgctcaccacgctctcgcaggcgatctcgggtcagctcaacccgaacgtcaatctcgtcgacacgttcaacggcggccagttcaccgtgttcgcgccgaccaatgacgccttcgccaagatcgatccggccacgctggagaccctcaagaccgattccgacctgctgaccaagatcctcacctaccacgtcgtgcccggccaggccgcgcccgatcaggtggtcggcgagcatgtgacggtggagggggcgccggtcacggtgtccgggatggccgaccagctcaaggtcaacgacgcgtcggtggtgtgcggtggggtgcagaccgccaacgcgacggtgtatctgatcgacaccgtgctgatgccgccggcagcgtagccgggcggcaccacagaagagggtcccccgcacccggcctcccccg

SEQ ID NO: 152 - protein, 231 aa, Mycobacterium vaccae
DTVLMPPANNRRSSTAGRNLTIMNISMKTLAGAGFAMTAAVGLSLGTAGSAAAAPVGPGCAAYVQQVPDGPGSVQGMASSPVATAAADNPLLTTLSQAISGQLNPNVNLVDTFNGGQFTVFAPTNDAFAKIDPATLETLKTDSDLLTKILTYHVVPGQAAPDQVVGEHVTVEGAPVTVSGMADQLKVNDASVVCGGVQTANATVYLIDTVLMPPAAPGGTTEEGPPHPASP

SEQ ID NO: 153 - DNA, 1125 bp, Mycobacterium vaccae
  unsure (358)...(358)
atgcaggtgcggcgtgttctgggcagtgtcggtgcagcagtcgcggtttcggccgcgttatggcagacgggggtttcgataccgaccgcctcagcggatccgtgtccggacatcgaggtgatcttcgcgcgcgggaccggtgcggaacccggcctcgggtgggtcggtgatgcgttcgtcaacgcgctgcggcccaaggtcggtgagcagtcggtgggcacctacgcggtgaactacccggcaggattcggacttcgacaaatcggcgcccatgggcgcggccgacgcatcggggcgggtgcagtggatggccgacaactgcccggacaccaagcttgtcctgggcggcatgtcgcanggcgccggcgtcatcgacctgatcaccgtcgatccgcgaccgctgggccggttcacccccaccccgatgccgccccgcgtcgccgaccacgtggccgccgttgtggtcttcggaaatccgttgcgcgacatccgtggtggcggtccgctgccgcagatgagcggcacctacgggccgaagtcgatcgatctgtgtgcgctcgacgatccgttctgctcgcccggcttcaacctgccggcccacttcgcctacgccgacaacggcatggtggaggaagccgcgaacttcgcccgcctggaaccgggccagagcgtcgagctgcccgaggcgccctacctgcacctgttcgtcccgcggggcgaggtaacgctggaggacgccggaccgctgcgcgaaggcgacgcagtgcgtttcaccgcatcgggcggccagcgggtgaccgccaccgcgcccgcggagatcctcgtctgggagatgcatgcgggactcggtgcggcataagcgaataggagtcctgctggccggcgcagcactgctcgccggatgcacatccgaacctggacccgggccgtcggcggcaccggccccgacgagcacaaccgagagcgcacccggtcccggactcgtcccggtgaccgtcgcggtcgacgaacctctggccgacgcgccgttcgaccagccccgggaggccctggtgccgcagggttggacgctgtcggtgtgggcgcggaccgcccggccgcggctggccgcgtgggccccggacg

SEQ ID NO: 154 - protein, 748 aa, Mycobacterium vaccae
  UNSURE (119)...(119)
MQVRRVLGSVGAAVAVSAALWQTGVSIPTASADPCPDIEVIFARGTGAEPGLGWVGDAFVNALRPKVGEQSVGTYAVNYPAGFDFDKSAPMGAADASGRVQWMADNCPDTKLVLGGMSXGAGVIDLITVDPRPLGRFTPTPMPPRVADHVAAVVVFGNPLRDIRGGGPRLEPRGLNMETSERGLYTHRTYRGLYPRLYSSERILEASPLECYSALALEASPASPPRPHECYSSERPRGLYPHEASNLEPRALAHISPHEALATYRALAASPASNGLYMETVALGLGLALAALAASNPHEALAARGLEGLPRGLYGLNSERVALGLLEPRGLALAPRTYRLEHISLEPHEVALPRARGGLYGLVALTHRLEGLASPALAGLYPRLEARGGLGLYASPALAVALARGPHETHRALASERGLYGLYGLNARGVALTHRALATHRALAPRALAGLILELEVALTRPGLMETHISALAGLYLEGLYALAALAALAASNARGSERPRALAGLYARGARGSERTHRALAARGARGMETHISILEARGTHRTRPTHRARGALAVALGLYGLYTHRGLYPRASPGLHISASNARGGLARGTHRARGSERARGTHRARGPRGLYASPARGARGGLYARGARGTHRSERGLYARGARGALAVALARGPRALAPRGLYGLYPRGLYALAALAGLYLEASPALAVALGLYVALGLYALAASPARGPRALAALAALAGLYARGVALGLYPRGLYARGPRGLY

SEQ ID NO: 155 - DNA, 666 bp, Mycobacterium vaccae
atgaaggcaaatcattcgggatgctacaaatccgccggcccgatatggtcgcatccatcgccgctttgttcgcccgcactggcaccatctcatgcaggtctggacaatgagctgagcctgggcatccacggccagggcccggaacgactgaccattcagcagtgggacaccttcctcaacggcgtcttcccgttggaccgcaaccggttgacccgggagtggttccactcgggcaaggcgacctacgtcgtggccggtgaaggtgccgacgagttcgagggcacgctggagctgggctaccaggtgggctttccgtggtcgctgggcgtgggcatcaacttcagctacaccaccccgaacatcacgtacgacggttacggcctcaacttcgccgacccgctgctgggcttcggtgattccatcgtgaccccgccgctgttcccgggtgtctcgatcacggcggacctgggcaacggccccggcatccaggaggtcgcgaccttctccgtggacgtggccggccccggtggttccgtggtggtgtccaacgcgcacggcacggtcaccggtgctgccggtggtgtgctgctgcgtccgttcgcccgcctgatctcgtcgaccggcgacagcgtcaccacctacggcgcaccctggaacatgaactga

SEQ ID NO: 156 - protein, 221 aa, Mycobacterium vaccae
MKANHSGCYKSAGPIWSHPSPLCSPALAPSHAGLDNELSLGVHGQGPEHLTIQQWDTFLNGVFPLDRNRLTREWFHSGKATYVVAGEGADEFEGTLELGYHVGFPWSLGVGINFSYTTPNITYDGYGLNFADPLLGFGDSIVTPPLFPGVSITADLGNGPGIQEVATFSVDVAGPGGSVVVSNAHGTVTGAAGGVLLRPFARLISSTGDSVTTYGAPWNMN

SEQ ID NO: 157 - DNA, 480 bp, Mycobacterium vaccae
aacggctgggacatcaacacccctgcgttcgagtggttctacgagtccggcttgtcgacgatcatgccggtcggcggacagtccagcttctacagcgactggtaccagccgtctcggggcaacgggcagaactacacctacaagtgggagacgttcctgacccaggagctgccgacgtggctggaggccaaccgcggagtgtcgcgcaccggcaacgcgttcgtcggcctgtcgatggcgggcagcgcggcgctgacctacgcgatccatcacccgcagcagttcatctacgcctcgtcgctgtcaggcttcctgaacccgtccgagggctggtggccgatgctgatcgggctggcgatgaacgacgcaggcggcttcaacgccgagagcatgtggggcccgtcctcggacccggcgtggaagcgcaacgacccgatggtcaacatcaaccagctggtggccaacaacacccggatctgg

SEQ ID NO: 158 - protein, 161 aa, Mycobacterium vaccae
NGWDINTPAFEWFYESGLSTIMPVGGQSSFYSDWYQPSRGNGQNYTYKWETFLTQELPTWLEANRGVSRTGNAFVGLSMAGSAALTYAIHHPQQFIYASSLSGFLNPSEGWWPMLIGLAMNDAGGFNAESMWGPSSDPAWKRNDPMVNINQLVANNTRIWI

SEQ ID NO: 159 - DNA, 1626 bp, Mycobacterium vaccae
atggccaagacaattgcgtatgacgaagaggcccgccgtggcctcgagcggggcctcaacgccctcgcagacgccgtaaaggtgacgttgggcccgaagggtcgcaacgtcgtgctggagaagaagtggggcgcccccacgatcaccaacgatggtgtgtccatcgccaaggagatcgagctggaggacccgtacgagaagatcggcgctgagctggtcaaagaggtcgccaagaagaccgacgacgtcgcgggcgacggcaccaccaccgccaccgtgctcgctcaggctctggttcgcgaaggcctgcgcaacgtcgcagccggcgccaacccgctcggcctcaagcgtggcatcgagaaggctgtcgaggctgtcacccagtcgctgctgaagtcggccaaggaggtcgagaccaaggagcagatttctgccaccgcggcgatttccgccggcgacacccagatcggcgagctcatcgccgaggccatggacaaggtcggcaacgagggtgtcatcaccgtcgaggagtcgaacaccttcggcctgcagctcgagctcaccgagggtatgcgcttcgacaagggctacatctcgggttacttcgtgaccgacgccgagcgccaggaagccgtcctggaggatccctacatcctgctggtcagctccaaggtgtcgaccgtcaaggatctgctcccgctgctggagaaggtcatccaggccggcaagccgctgctgatcatcgccgaggacgtcgagggcgaggccctgtccacgctggtggtcaacaagatccgcggcaccttcaagtccgtcgccgtcaaggctccgggcttcggtgaccgccgcaaggcgatgctgcaggacatggccatcctcaccggtggtcaggtcgtcagcgaaagagtcgggctgtccctggagaccgccgacgtctcgctgctgggccaggcccgcaaggtcgtcgtcaccaaggacgagaccaccatcgtcgagggctcgggcgattccgatgccatcgccggccgggtggctcagatccgcgccgagatcgagaacagcgactccgactacgaccgcgagaagctgcaggagcgcctggccaagctggccggcggtgttgcggtgatcaaggccggagctgccaccgaggtggagctcaaggagcgcaagcaccgcatcgaggacgccgtccgcaacgcgaaggctgccgtcgaagagggcatcgtcgccggtggcggcgtggctctgctgcagtcggctcctgcgctggacgacctcggcctgacgggcgacgaggccaccggtgccaacatcgtccgcgtggcgctgtcggctccgctcaagcagatcgccttcaacggcggcctggagcccggcgtcgttgccgagaaggtgtccaacctgcccgcgggtcacggcctcaacgccgcgaccggtgagtacgaggacctgctcaaggccggcgtcgccgacccggtgaaggtcacccgctcggcgctgcagaacgcggcgtccatcgcggctctgttcctcaccaccgaggccgtcgtcgccgacaagccggagaaggcgtccgcacccgcgggcgacccgaccggtggcatgggcggtatggacttctaa

SEQ ID NO: 160 - protein, 541 aa, Mycobacterium vaccae
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEAVTQSLLKSAKEVETKEQISATAAISAGDTQIGELIAEAMDKVGNEGVITVEESNTFGLQLELTEGMRFDKGYISGYFVTDAERQEAVLEDPYILLVSSKVSTVKDLLPLLEKVIQAGKPLLIIAEDVEGEALSTLVVNKIRGTFKSVAVKAPGFGDRRKAMLQDMAILTGGQVVSERVGLSLETADVSLLGQARKVVVTKDETTIVEGSGDSDAIAGRVAQIRAEIENSDSDYDREKLQERLAKLAGGVAVIKAGAATEVELKERKHRIEDAVRNAKAAVEEGIVAGGGVALLQSAPALDDLGLTGDEATGANIVRVALSAPLKQIAFNGGLEPGVVAEKVSNLPAGHGLNAATGEYEDLLKAGVADPVKVTRSALQNAASIAALFLTTEAVVADKPEKASAPAGDPTGGMGGMDF

SEQ ID NO: 161 - DNA, 985 bp, Mycobacterium vaccae
ggatccctacatcctgctggtcagctccaaggtgtcgaccgtcaaggatctgctcccgctgctggagaaggtcatccaggccggcaagccgctgctgatcatcgccgaggacgtcgagggcgaggccctgtccacgctggtggtcaacaagatccgcggcaccttcaagtccgtcgccgtcaaggctccgggcttcggtgaccgccgcaaggcgatgctgcaggacatggccatcctcaccggtggtcaggtcgtcagcgaaagagtcgggctgtccctggagaccgccgacgtctcgctgctgggccaggcccgcaaggtcgtcgtcaccaaggacgagaccaccatcgtcgagggctcgggcgattccgatgccatcgccggccgggtggctcagatccgcgccgagatcgagaacagcgactccgactacgaccgcgagaagctgcaggagcgcctggccaagctggccggcggtgttgcggtgatcaaggccggagctgccaccgaggtggagctcaaggagcgcaagcaccgcatcgaggacgccgtccgcaacgcgaaggctgccgtcgaagagggcatcgtcgccggtggcggcgtggctctgctgcagtcggctcctgcgctggacgacctcggcctgacgggcgacgaggccaccggtgccaacatcgtccgcgtggcgctgtcggctccgctcaagcagatcgccttcaacggcggcctggagcccggcgtcgttgccgagaaggtgtccaacctgcccgcgggtcacggcctcaacgccgcgaccggtgagtacgaggacctgctcaaggccggcgtcgccgacccggtgaaggtcacccgctcggcgctgcagaacgcggcgtccatcgcggctctgttcctcaccaccgaggccgtcgtcgccgacaagccggagaaggcgtccgcacccgcgggcgacccgaccggtggcatgggcggtatggacttctaa

SEQ ID NO: 162 - protein, 327 aa, Mycobacterium vaccae
DPYILLVSSKVSTVKDLLPLLEKVIQAGKPLLIIAEDVEGEALSTLVVNKIRGTFKSVAVKAPGFGDRRKAMLQDMAILTGGQVVSERVGLSLETADVSLLGQARKVVVTKDETTIVEGSGDSDAIAGRVAQIRAEIENSDSDYDREKLQERLAKLAGGVAVIKAGAATEVELKERKHRIEDAVRNAKAAVEEGIVAGGGVALLQSAPALDDLGLTGDEATGANIVRVALSAPLKQIAFNGGLEPGVVAEKVSNLPAGHGLNAATGEYEDLLKAGVADPVKVTRSALQNAASIAALFLTTEAVVADKPEKASAPAGDPTGGMGGMDF

SEQ ID NO: 163 - DNA, 403 bp, Mycobacterium vaccae
ggatccgcggcaccggctggtgacgaccaagtacaacccggcccgcacctggacggccgagaactccgtcggcatcggcggcgcgtacctgtgcatctacgggatggagggccccggcggctatcagttcgtcggccgcaccacccaggtgtggagtcgttaccgccacacggcgccgttcgaacccggaagtccctggctgctgcggtttttcgaccgaatttcgtggtatccggtgtcggccgaggagctgctggaattgcgagccgacatggccgcaggccggggctcggtcgacatcaccgacggcgtgttctccctcgccgagcacgaacggttcctggccgacaacgccgacgacatcgccgcgttccgttcccggcaggcggccgcgttctccgcc

SEQ ID NO: 164 - DNA, 336 bp, Mycobacterium vaccae
cggaccgcgtgggcggccgccggcgagttcgaccgcgccgagaaagccgcgtcgaaggccaccgacgccgataccggggacctggtgctctacgacggtgcgagcgggtcgacgctccgttcgcgtcgagcgtgtggaaggtcgacgtcgccgtcggtgaccgggtggtggccggacagccgttgctggcgctggaggcgatgaagatggagaccgtgctgcgcgccccggccgacggggtggtcacccagatcctggtctccgctgggcatctcgtcgatcccggcaccccactggtcgtggtcggcaccggagtgcgcgcatgagcgccgtcga

SEQ ID NO: 165 - protein, 134 aa, Mycobacterium vaccae
DPRHRLVTTKYNPARTWTAENSVGIGGAYLCIYGMEGPGGYQFVGRTTQVWSRYRHTAPFEPGSPWLLRFFDRISWYPVSAEELLELRADMAAGRGSVDITDGVFSLAEHERFLADNADDIAAFRSRQAAAFSA

SEQ ID NO: 166 - protein, 108 aa, Mycobacterium vaccae
RTAWAAAGEFDRAEKAASKATDADTGDLVLYDGDERVDAPFASSVWKVDVAVGDRVVAGQPLLALEAMKMETVLRAPADGVVTQILVSAGHLVDPGTPLVVVGTGVRA

SEQ ID NO: 167 - DNA, 31 bp, Artificial Sequence
  Made in a lab
atagaattcgtccgacagtgggacctcgagc

SEQ ID NO: 168 - DNA, 27 bp, Artificial Sequence
  Made in a lab
atagaattcccaccgcgtcagccgccg

SEQ ID NO: 169 - DNA, 1111 bp, Mycobacterium vaccae
gtccgacagtgggacctcgagcaccacgtcacaggacagcggccccgccagcggcgccctgcgcgtctccaactggccgctctatatggccgacggtttcatcgcagcgttccagaccgcctcgggcatcacggtcgactacaaagaagacttcaacgacaacgagcagtggttcgccaaggtcaaggagccgttgtcgcgcaagcaggacataggcgccgacctggtgatccccaccgagttcatggccgcgcgcgtcaagggcctgggatggctcaatgagatcagcgaagccggcgtgcccaatcgcaagaatctgcgtcaggacctgttggactcgagcatcgacgagggccgcaagttcaccgcgccgtacatgaccggcatggtcggtctcgcctacaacaaggcagccaccggacgcgatatccgcaccatcgacgacctctgggatcccgcgttcaagggccgcgtcagtctgttctccgacgtccaggacggcctcggcatgatcatgctctcgcagggcaactcgccggagaatccgaccaccgagtccattcagcaggcggtcgatctggtccgcgaacagaacgacagggggtcagatccgtcgcttcaccggcaacgactacgccgacgacctggccgcagaaacatcgccatcgcgcaggcgtactccggtgacgtcgtgcagctgcaggcggacaaccccgatctgcagttcatcgttcccgaatccggcggcgactggttcgtcgacacgatggtgatcccgtacaccacgcagaaccagaaggccgccgaggcgtggatcgactacatctacgaccgagccaactacgccaagctggtcgcgttcacccagttcgtgcccgcactctcggacatgaccgacgaactcgccaaggtcgatcctgcatcggcggagaacccgctgatcaacccgtcggccgaggtgcaggcgaacctgaagtcgtgggcggcactgaccgacgagcagacgcaggagttcaacactgcgtacgccgccgtcaccggcggctgacgcggtggtagtgccgatgcgaggggcataaatggccctgcggacgcgaggagcataaatggc

SEQ ID NO: 170 - protein, 348 aa, Mycobacterium vaccae
SDSGTSSTTSQDSGPASGALRVSNWPLYMADGFIAAFQTASGITVDYKEDFNDNEQWFAKVKEPLSRKQDIGADLVIPTEFMAARVKGLGWLNEISEAGVPNRKNLRQDLLDSSIDEGRKFTAPYMTGMVGLAYNKAATGRDIRTIDDLWDPAFKGRVSLFSDVQDGLGMIMLSQGNSPENPTTESIQQAVDLVREQNDRGQIRRFTGNDYADDLAAGNIAIAQAYSGDVVQLQADNPDLQFIVPESGGDWFVDTMVIPYTTQNQKAAEAWIDYIYDRANYAKLVAFTQFVPALSDMTDELAKVDPASAENPLINPSAEVQANLKSWAALTDEQTQEFNTAYAAVTGG

SEQ ID NO: 171 - DNA, 1420 bp, Mycobacterium vaccae
  unsure (955)...(955)
  unsure (973)...(973)
gatgagcagcgtgctgaactcgacctggttggcctgggccgtcgcggtcgcggtcgggttcccggtgctgctggtcgtgctgaccgaggtgcacaacgcgttgcgtcggcgcggcagcgcgctggcccgcccggtgcaactcctgcgtacctacatcctgccgctgggcgcgttgctgctcctgctggtacaggcgatggagatctccgacgacgccacgtcggtacggttggtcgccaccctgttcggcgtcgtgttgttgacgttggtgctgtccgggctcaacgccaccctcatccagggcgcaccagaagacagctggcgcaggcggattccgtcgatcttcctcgacgtcgcgcgcttcgcgctgatcgcggtcggtatcaccgtgatcatggcctatgtctggggcgcgaacgtggggggcctgttcaccgcactgggcgtcacttccatcgttcttggcctggctctgcagaattcggtcggtcagatcatctcgggtctgctgctgctgttcgagcaaccgttccggctcggcgactggatcaccgtccccaccgcggcgggccggccgtccgcccacggccgcgtggtggaagtcaactggcgtgcaacacatatcgacaccggcggcaacctgctggtaatgcccaacgccgaactcgccggcgcgtcgttcaccaattacagccggcccgtgggagagcaccggctgaccgtcgtcaccaccttcaacgccgcggacacccccgatgatgtctgcgagatgctgtcgtcggtcgcggcgtcgctgcccgaactgcgcaccgacggacagatcgccacgctctatctcggtgcggccgaatacgagaagtcgatcccgttgcacacacccgcggtggacgactcggtcaggagcacgtacctgcgatgggtctggtacgccgcgcgccggcaggaacttcgcctnaacggcgtcgccgacganttcgacacgccggaacggatcgcctcggccatgcgggctgtggcgtccacactgcgcttggcagacgacgaacagcaggagatcgccgacgtggtgcgtctggtccgttacggcaacggggaacgcctccagcagccgggtcaggtaccgaccgggatgaggttcatcgtagacggcagggtgagtctgtccgtgatcgatcaggacggcgacgtgatcccggcgcgggtgctcgagcgtggcgacttcctggggcagaccacgctgacgcgggaaccggtactggcgaccgcgcacgcgctggaggaagtcaccgtgctggagatggcccgtgacgagatcgagcgcctggtgcaccgaaagccgatcctgctgcacgtgatcggggccgtgatcgccgaccggcgcgcgcacgaacttcggttgatggcggactcgcaggactga

SEQ ID NO: 172 - protein, 471 aa, Mycobacterium vaccae
  UNSURE (318)...(318)
  UNSURE (324)...(324)
MSSVLNSTWLAWAVAVAVGFPVLLVVLTEVHNALRRRGSALARPVQLLRTYILPLGALLLLLVQAMEISDDATSVRLVATLFGVVLLTLVLSGLNATLIQGAPEDSWRRRIPSIFLDVARFALIAVGITVIMAYVWGANVGGLFTALGVTSIVLGLALQNSVGQIISGLLLLFEQPFRLGDWITVPTAAGRPSAHGRVVEVNWRATHIDTGGNLLVMPNAELAGASFTNYSRPVGEHRLTVVTTFNAADTPDDVCEMLSSVAASLPELRTDGQIATLYLGAAEYEKSIPLHTPAVDDSVRSTYLRWVWYAARRQELRXNGVADXFDTPERIASAMRAVASTLRLADDEQQEIADVVRLVRYGNGERLQQPGQVPTGMRFIVDGRVSLSVIDQDGDVIPARVLERGDFLGQTTLTREPVLATAHALEEVTVLEMARDEIERLVHRKPILLHVIGAVIADRRAHELRLMDSQD

SEQ ID NO: 173 - DNA, 2172 bp, Mycobacterium vaccae
tagatgacaattctgccctggaatgcgcgaacgtctgaacacccgacgcgaaaaagacgcgggcgctaccacctcctgtcgcggatgagcatccagtccaagttgctgctgatgctgcttctgaccagcattctctcggctgcggtggtcggtttcatcggctatcagtccggacggtcctcgctgcgcgcatcggtgttcgaccgcctcaccgacatccgcgagtcgcagtcgcgcgggttggagaatcagttcgcggacctgaagaactcgatggtgatttactcgcgcggcagcactgccacggaggcgatcggcgcgttcagcgacggtttccgtcagctcggcgatgcgacgatcaataccgggcaggcggcgtcattgcgccgttactacgaccggacgttcgccaacaccaccctcgacgacagcggaaaccgcgtcgacgtccgcgcgctcatcccgaaatccaacccccagcgctatctgcaggcgctctataccccgccgtttcagaactgggagaaggcgatcgcgttcgacgacgcgcgcgacggcagcgcctggtcggccgccaatgccagattcaacgagttcttccgcgagatcgtgcaccgcttcaacttcgaggatctgatgctgctcgacctcgagggcaacgtggtgtactccgcctacaaggggccggatctcgggacaaacatcgtcaacggcccctatcgcaaccgggaactgtcggaagcctacgagaaggcggtcgcgtcgaactcgatcgactatgtcggtgtcaccgacttcgggtggtacctgcctgccgaggaaccgaccgcctggttcctgtccccggtcgggttgaaggaccgagtcgacggtgtgatggcggtccagttcccgatcgcgcggatcaacgaattgatgacggcgcggggacagtggcgtgacaccgggatgggagacaccggtgagaccatcctggtcggaccggacaatctgatgcgctcggactcccggctgttccgcgagaaccgggagaagttcctggccgacgtcgtcgaggggggaaccccgccggaggtcgccgacgaatcggttgaccgccgcggcaccacgctggtgcagccggtgaccacccgctccgtcgaggaggcccaacgcggcaacaccgggacgacgatcgaggacgactatctcggccacgaggcgttacaggcgtactcaccggtggacctgccgggactgcactgggtgatcgtggccaagatcgacaccgacgaggcgttcgccccggtggcgcagttcaccaggaccctggtgctgtcgacggtgatcatcatcttcggcgtgtcgctggcggccatgctgctggcgcggttgttcgtccgtccgatccggcggttgcaggccggcgcccagcagatcagcggcggtgactaccgcctcgctctgccggtgttgtctcgtgacgaattcggcgatctgacaacagctttcaacgacatgagtcgcaatctgtcgatcaaggacgagctgctcggcgaggagcgcgccgagaaccaacggctgatgctgtccctgatgcccgaaccggtgatgcagcgctacctcgacggggaggagacgatcgcccaggaccacaagaacgtcacggtgatcttcgccgacatgatgggcctcgacgagttgtcgcgcatgttgacctccgaggaactgatggtggtggtcaacgacctgacccgccagttcgacgccgccgccgagagtctcggggtcgaccacgtgcggacgctgcacgacgggtacctggccagctgcgggttaggcgtgccgcggctggacaacgtccggcgcacggtcaatttcgcgatcgaaatggaccgcatcatcgaccggcacgccgccgagtccgggcacgacctgcggctccgcgcgggcatcgacaccgggtcggcggccagcgggctggtggggcggtccacgttggcgtacgacatgtggggttcggcggtcgatgtcgctaaccaggtgcagcgcggctccccccagcccggcatctacgtcacctcgcgggtgcacgaggtcatgcaggaaactctcgacttcgtcgccgccggggaggtcgtcggcgagcgcggcgtcgagacggtctggcggttgcagggccaccggcgatga

SEQ ID NO: 174 - protein, 722 aa, Mycobacterium vaccae
MTILPWNARTSEHPTRKRRGRYHLLSRMSIQSKLLLMLLLTSILSAAVVGFIGYQSGRSSLRASVFDRLTDIRESQSRGLENQFADLKNSMVIYSRGSTATEAIGAFSDGFRQLGDATINTGQAASLRRYYDRTFANTTLDDSGNRVDVRALIPKSNPQRYLQALYTPPFQNWEKAIAFDDARDGSAWSAANARFNEFFREIVHRFNFEDLMLLDLEGNVVYSAYKGPDLGTNIVNGPYRNRELSEAYEKAVASNSIDYVGVTDFGWYLPAEEPTAWFLSPVGLKDRVDGVMAVQFPIARINELMTARGQWRDTGMGDTGETILVGPDNLMRSDSRLFRENREKFLADVVEGGTPPEVADESVDRRGTTLVQPVTTRSVEEAQRGNTGTTIEDDYLGHEALQAYSPVDLPGLHWVIVAKIDTDEAFAPVAQFTRTLVLSTVIIIFGVSLAAMLLARLFVRPIRRLQAGAQQISGGDYRLALPVLSRDEFGDLTTAFNDMSRNLSIKDELLGEERAENQRLMLSLMPEPVMQRYLDGEETIAQDHKNVTVIFADMMGLDELSRMLTSEELMVVVNDLTRQFDAAAESLGVDHVRTLHDGYLASCGLGVPRLDNVRRTVNFAIEMDRIIDRHAAESGHDLRLRAGIDTGSAASGLVGRSTLAYDMWGSAVDVANQVQRGSPQPGIYVTSRVHEVMQETLDFVAAGEVVGERGVETVWRLQGHRR

SEQ ID NO: 175 - DNA, 898 bp, Mycobacterium vaccae
gagcaaccgttccggctcggcgactggatcaccgtccccaccgcggcgggccggccgtccgcccacggccgcgtggtggaagtcaactggcgtgcaacacatatcgacaccggcggcaacctgctggtaatgcccaacgccgaactcgccggcgcgtcgttcaccaattacagccggcccgtgggagagcaccggctgaccgtcgtcaccaccttcaacgccgcggacacccccgatgatgtctgcgagatgctgtcgtcggtcgcggcgtcgctgcccgaactgcgcaccgacggacagatcgccacgctctatctcggtgcggccgaatacgagaagtcgatcccgttgcacacacccgcggtggacgactcggtcaggagcacgtacctgcgatgggtctggtacgccgcgcgccggcaggaacttcgcctaacggcgtcgccgacgattcgacacgccggaacggatcgcctcggccatgcgggctgtggcgtccacactgcgcttggcagacgacgaacagcaggagatcgccgacgtggtgcgtctggtccgttacggcaacggggaacgcctccagcagccgggtcaggtaccgaccgggatgaggttcatcgtagacggcagggtgagtctgtccgtgatcgatcaggacggcgacgtgatcccggcgcgggtgctcgagcgtggcgacttcctggggcagaccacgctgacgcgggaaccggtactggcgaccgcgcacgcgctggaggaagtcaccgtgctggagatggcccgtgacgagatcgagcgcctggtgcaccgaaagccgatcctgctgcacgtgatcggggccgtgatcgccgaccggcgcgcgcacgaacttcggttgatggcggactcgcaggactga

SEQ ID NO: 176 - DNA, 2013 bp, Mycobacterium vaccae
ggctatcagtccggacggtcctcgctgcgcgcatcggtgttcgaccgcctcaccgacatccgcgagtcgcagtcgcgcgggttggagaatcagttcgcggacctgaagaactcgatggtgatttactcgcgcggcagcactgccacggaggcgatcggcgcgttcagcgacggtttccgtcagctcggcgatgcgacgatcaataccgggcaggcggcgtcattgcgccgttactacgaccggacgttcgccaacaccaccctcgacgacagcggaaaccgcgtcgacgtccgcgcgctcatcccgaaatccaacccccagcgctatctgcaggcgctctataccccgccgtttcagaactgggagaaggcgatcgcgttcgacgacgcgcgcgacggcagcgcctggtcggccgccaatgccagattcaacgagttcttccgcgagatcgtgcaccgcttcaacttcgaggatctgatgctgctcgacctcgagggcaacgtggtgtactccgcctacaaggggccggatctcgggacaaacatcgtcaacggcccctatcgcaaccgggaactgtcggaagcctacgagaaggcggtcgcgtcgaactcgatcgactatgtcggtgtcaccgacttcgggtggtacctgcctgccgaggaaccgaccgcctggttcctgtccccggtcgggttgaaggaccgagtcgacggtgtgatggcggtccagttcccgatcgcgcggatcaacgaattgatgacggcgcggggacagtggcgtgacaccgggatgggagacaccggtgagaccatcctggtcggaccggacaatctgatgcgctcggactcccggctgttccgcgagaaccgggagaagttcctggccgacgtcgtcgaggggggaaccccgccggaggtcgccgacgaatcggttgaccgccgcggcaccacgctggtgcagccggtgaccacccgctccgtcgaggaggcccaacgcggcaacaccgggacgacgatcgaggacgactatctcggccacgaggcgttacaggcgtactcaccggtggacctgccgggactgcactgggtgatcgtggccaagatcgacaccgacgaggcgttcgccccggtggcgcagttcaccaggaccctggtgctgtcgacggtgatcatcatcttcggcgtgtcgctggcggccatgctgctggcgcggttgttcgtccgtccgatccggcggttgcaggccggcgcccagcagatcagcggcggtgactaccgcctcgctctgccggtgttgtctcgtgacgaattcggcgatctgacaacagctttcaacgacatgagtcgcaatctgtcgatcaaggacgagctgctcggcgaggagcgcgccgagaaccaacggctgatgctgtccctgatgcccgaaccggtgatgcagcgctacctcgacggggaggagacgatcgcccaggaccacaagaacgtcacggtgatcttcgccgacatgatgggcctcgacgagttgtcgcgcatgttgacctccgaggaactgatggtggtggtcaacgacctgacccgccagttcgacgccgccgccgagagtctcggggtcgaccacgtgcggacgctgcacgacgggtacctggccagctgcgggttaggcgtgccgcggctggacaacgtccggcgcacggtcaatttcgcgatcgaaatggaccgcatcatcgaccggcacgccgccgagtccgggcacgacctgcggctccgcgcgggcatcgacaccgggtcggcggccagcgggctggtggggcggtccacgttggcgtacgacatgtggggttcggcggtcgatgtcgctaaccaggtgcagcgcggctccccccagcccggcatctacgtcacctcgcgggtgcacgaggtcatgcaggaaactctcgacttcgtcgccgccggggaggtcgtcggcgagcgcggcgtcgagacggtctggcggttgcagggccaccggcgatga

SEQ ID NO: 177 - protein, 297 aa, Mycobacterium vaccae
  UNSURE (145)...(145)
  UNSURE (151)...(151)
EQPFRLGDWITVPTAAGRPSAHGRVVEVNWRATHIDTGGNLLVMPNAELAGASFTNYSRPVGEHRLTVVTTFNAADTPDDVCEMLSSVAASLPELRTDGQIATLYLGAAEYEKSIPLHTPAVDDSVRSTYLRWVWYAARRQELRXNGVADXFDTPERIASAMRAVASTLRLADDEQQEIADVVRLVRYGNGERLQQPGQVPTGMRFIVDGRVSLSVIDQDGDVIPARVLERGDFLGQTTLTREPVLATAHALEEVTVLEMARDEIERLVHRKPILLHVIGAVADRRAHELRLMDSQD

SEQ ID NO: 178 - protein, 670 aa, Mycobacterium vaccae
GYQSGRSSLRASVFDRLTDIRESQSRGLENQFADLKNSMVIYSRGSTATEAIGAFSDGFRQLGDATINTGQAASLRRYYDRTFANTTLDDSGNRVDVRALIPKSNPQRYLQALYTPPFQNWEKAIAFDDARDGSAWSAANARFNEFFREIVHRFNFEDLMLLDLEGNVVYSAYKGPDLGTNIVNGPYRNRELSEAYEKAVASNSIDYVGVTDFGWYLPAEEPTAWFLSPVGLKDRVDGVMAVQFPIARINELMTARGQWRDTGMGDTGETILVGPDNLMRSDSRLFRENREKFLADVVEGGTPPEVADESVDRRGTTLVQPVTTRSVEEAQRGNTGTTIEDDYLGHEALQAYSPVDLPGLHWVIVAKIDTDEAFAPVAQFTRTLVLSTVIIIFGVSLAAMLLARLFVRPIRRLQAGAQQISGGDYRLALPVLSRDEFGDLTTAFNDMSRNLSIKDELLGEERAENQRLMLSLMPEPVMQRYLDGEETIAQDHKNVTVIFADMMGLDELSRMLTSEELMVVVNDLTRQFDAAAESLGVDHVRTLHDGYLASCGLGVPRLDNVRRTVNFAIEMDRIIDRHAAESGHDLRLRAGIDTGSAASGLVGRSTLAYDMWGSAVDVANQVQRGSPQPGIYVTSRVHEVMQETLDFVAAGEVVGERGVETVWRLQGHRR

SEQ ID NO: 179 - DNA, 520 bp, Mycobacterium vaccae
gtgatcgacgaaaccctcttccatgccgaggagaagatggagaaggccgtctcggtggcacccgacgacctggcgtcgattcgtaccggccgcgcgaaccccggcatgttcaaccggatcaacatcgactactacggcgcctccaccccgatcacgcagctgtccagcatcaacgtgcccgaggcgcgcatggtggtgatcaagccctacgaggcgagccagctgcgcctcatcgaggatgcgatccgcaactccgacctcggcgtcaatccgaccaacgacggcaacatcatccgggtgtcgatcccgcagctcaccgaggagcgccgccgcgacctggtcaagcaggccaaggccaagggcgaggacgccaaggtgtcggtgcgcaacatccgtcgcaacgatatgaacacctttcgcatcgcaccggtacggctgccgacgccaccgccgtcgtagaagcgacagaggatcgcaggtaacggtattggccacgccttctgtggcgggccgacaccac

SEQ ID NO: 180 - DNA, 1071 bp, Mycobacterium vaccae
cgtggggaaggattgcactctatgagcgaaatcgcccgtccctggcgggttctggcaggtggcatcggtgcctgcgccgcgggtatcgccggggtgctgagcatcgcggtcaccacggcgtcggcccagccgggcctcccgcagcccccgctgcccgcccctgccacagtgacgcaaaccgtcacggttgcgcccaacgccgcgccacaactcatcccgcgccccggtgtgacgcctgccaccggcggcgccgccgcggtgcccgccggggtgagcgccccggcggtcgcgccggcccccgcgctgcccgcccgcccggtgtccacgatcgccccggccacctcgggcacgctcagcgagttcttcgccgccaagggcgtcacgatggagccgcagtccagccgcgacttccgcgccctcaacatcgtgctgccgaagccgcggggctgggagcacatcccggacccgaacgtgccggacgcgttcgcggtgctggccgaccgggtcggcggcaacggcctgtactcgtcgaacgcccaggtggtggtctacaaactcgtcggcgagttcgaccccaaggaagcgatcagccacggcttcgtcgacagccagaagctgccggcgtggcgttccaccgacgcgtcgctggccgacttcggcggaatgccgtcctcgctgatcgagggcacctaccgcgagaacaacatgaagctgaacacgtcccggcgccacgtcattgccaccgcggggcccgaccactacctggtgtcgctgtcggtgaccaccagcgtcgaacaggccgtggccgaagccgcggaggccaccgacgcgattgtcaacggcttcaaggtcagcgttccgggtccgggtccggccgcaccgccacctgcacccggtgcccccggtgtcccgcccgcccccggcgccccggcgctgccgctggccgtcgcaccacccccggctcccgctgttcccgccgtggcgcccgcgccacagctgctgggactgcagggatagacgtcgtcgtcccccgggcgaagcctggcgcccgggggacgacggcccctttct

SEQ ID NO: 181 - protein, 152 aa, Mycobacterium vaccae
VIDETLFHAEEKMEKAVSVAPDDLASIRTGRANPGMFNRINIDYYGASTPITQLSSINVPEARMVVIKPYEASQLRLIEDAIRNSDLGVNPTNDGNIIRVSIPQLTEERRRDLVKQAKAKGEDAKVSVRNIRRNDMNTFRIAPVRLPTPPPS

SEQ ID NO: 182 - protein, 331 aa, Mycobacterium vaccae
MSEIARPWRVLAGGIGACAAGIAGVLSIAVTTASAQPGLPQPPLPAPATVTQTVTVAPNAAPQLIPRPGVTPATGGAAAVPAGVSAPAVAPAPALPARPVSTIAPATSGTLSEFFAAKGVTMEPQSSRDFRALNIVLPKPRGWEHIPDPNVPDAFAVLADRVGGNGLYSSNAQVVVYKLVGEFDPKEAISHGFVDSQKLPAWRSTDASLADFGGMPSSLIEGTYRENNMKLNTSRRHVIATAGPDHYLVSLSVTTSVEQAVAEAAEATDAIVNGFKVSVPGPGPAAPPPAPGAPGVPPAPGAPALPLAVAPPPAPAVPAVAPAPQLLGLQG

SEQ ID NO: 183 - DNA, 207 bp, Mycobacterium vaccae
acctacgagttcgagaacaaggtcacgggcggccgcatcccgcgcgagtacatcccgtcggtggatgccggcgcgcaggacgccatgcagtacggcgtgctggccggctacccgctggttaacgtcaagctgacgctgctcgacggtgcctaccacgaagtcgactcgtcggaaatggcattcaaggttgccggctcccaggtcata

SEQ ID NO: 184 - protein, 69 aa, Mycobacterium vaccae
TYEFENKVTGGRIPREYIPSVDAGAQDAMQYGVLAGYPLVNVKLTLLDGAYHEVDSSEMAFKVAGSQVI

SEQ ID NO: 185 - DNA, 898 bp, Mycobacterium vaccae
  unsure (637)...(637)
  unsure (662)...(662)
cgacctccacccgggcgtgaggccaaccactaggctggtcaccagtagtcgacggcacacttcaccgaaaaaatgaggacagaggagacacccgtgacgatccgtgttggtgtgaacggcttcggccgtatcggacgcaacttcttccgcgcgctggacgcgcagaaggccgaaggcaagaacaaggacatcgagatcgtcgcggtcaacgacctcaccgacaacgccacgctggcgcacctgctgaagttcgactcgatcctgggccggctgccctacgacgtgagcctcgaaggcgaggacaccatcgtcgtcggcagcaccaagatcaaggcgctcgaggtcaaggaaggcccggcggcgctgccctggggcgacctgggcgtcgacgtcgtcgtcgagtccaccggcatcttcaccaagcgcgacaaggcccagggccacctcgacgcgggcgccaagaaggtcatcatctccgcgccggccaccgatgaggacatcaccatcgtgctcggcgtcaacgacgacaagtacgacggcagccagaacatcatctccaacgcgtcgtgcaccacgaactgcctcggcccgctggcgaaggtcatcaacgacgagttcggcatcgtcaagggcctgntgaccaccatccacgcctacacccnggtccagaacctgcaggacggcccgcacaaggatctgcgccgggcccgcgccgccgcgctgaacatcgtgccgacctccaccggtgccgccaaggccatcggactggtgctgcccgagctgaagggcaagctcgacggctacgcgctgcgggtgccgatccccaccggctcggtcaccgacctgaccgccgagctgggcaagtcggccaccgtggacgagatcaacgccgcgatga

SEQ ID NO: 186 - protein, 268 aa, Mycobacterium vaccae
  UNSURE (182)...(182)
  UNSURE (190)...(190)
VTIRVGVNGFGRIGRNFFRALDAQKAEGKNKDIEIVAVNDLTDNATLAHLLKFDSILGRLPYDVSLEGEDTIVVGSTKIKALEVKEGPAALPWGDLGVDVVVESTGIFTKRDKAQGHLDAGAKKVIISAPATDEDITIVLGVNDDKYDGSQNIISNASCTTNCLGPLAKVINDEFGIVKGLXTTIHAYTXVQNLQDGPHKDLRRARAAALNIVPTSTGAAKAIGLVLPELKGKLDGYALRVPIPTGSVTDLTAELGKSATVDEINAAM

SEQ ID NO: 187 - protein, 41 aa, Mycobacterium vaccae
  UNSURE (39)...(39)
MNKAELIDVLTEKLGSDRRQATAAVENVVDTIVAAVPKXVV

SEQ ID NO: 188 - DNA, 26 bp, Artificial Sequence
  Made in a lab
  unsure (12)...(12)
atgaayaargcngarctsatygaygt

SEQ ID NO: 189 - DNA, 20 bp, Artificial Sequence
  Made in a lab
atsgtrtgvacvacgttytc

SEQ ID NO: 190 - DNA, 84 bp, Artificial Sequence
  Made in a lab
  unsure (2)...(2)
gnactcattgacgtactcactgagaagctgggctcggattgtcggcaagcgactgcggcaatggagaacgtggtccacaccata

SEQ ID NO: 191 - DNA, 337 bp, Mycobacterium vaccae
  unsure (2)...(2)
gnactcattgacgtactcactgagaagctgggctcggattgtcggcaagcgactgcggcggtggagaatgttgtcgacaccatcgtgcgcgccgtgcacaagggtgagagcgtcaccatcacgggcttcggtgttttcgagcagcgtcgtcgcgcagcacgcgtggcacgcaatccgcgcaccggcgagaccgtgaaggtcaagcccacctcagtcccggcattccgtcccggcgctcagttcaaggctgttgtctctggcgcacagaagcttccggccgagggtccggcggtcaagcgcggtgtgaccgcgacgagcaccgcccgcaaggcagcca

SEQ ID NO: 192 - protein, 111 aa, Mycobacterium vaccae
  UNSURE (1)...(1)
XLIDVLTEKLGSDRQATAAVENVVDTIVRAVHKGESVTITGFGVFEQRRRAARVARNPRTGETVKVKPTSVPAFRPGAQFKAVVSGAQKLPAEGPAVKRGVTATSTARKAA

SEQ ID NO: 193 - DNA, 1164 bp, Mycobacterium vaccae
ggtggcgcgcatcgagaagcgcccgccccggttcacgggcgcctgatcatggtgcgggcggcgctgcgctacggcttcgggacggcctcactgctggccggcgggttcgtgctgcgcgccctgcagggcacgcctgccgccctcggcgcgactccgggcgaggtcgcgccggtggcgcgccgctcgccgaactaccgcgacggcaagttcgtcaacctggagcccccgtcgggcatcacgatggatcgcgacctgcagcggatgctgttgcgcgatctggccaacgccgcatcccagggcaagccgcccggaccgatcccgctggccgagccgccgaagggggatcccactcccgcgccggcggcggccagctggtacggccattccagcgtgctgatcgaggtcgacggctaccgcgtgctggccgacccggtgtggagcaacagatgttcgccctcacgggcggtcggaccgcagcgcatgcacgacgtcccggtgccgctggaggcgcttcccgccgtggacgcggtggtgatcagccacgaccactacgaccacctcgacatcgacaccatcgtcgcgttggcgcacacccagcgggccccgttcgtggtgccgttgggcatcggcgcacacctgcgcaagtggggcgtccccgaggcgcggatcgtcgagttggactggcacgaagcccaccgcatagacgacctgacgctggtctgcacccccgcccggcacttctccggacggttgttctcccgcgactcgacgctgtgggcgtcgtgggtggtcaccggctcgtcgcacaaggcgttcttcggtggcgacaccggatacacgaagagcttcgccgagatcggcgacgagtacggtccgttcgatctgaccctgctgccgatcggggcctaccatcccgcgttcgccgacatccacatgaaccccgaggaggcggtgcgcgcccatctggacctgaccgaggtggacaacagcctgatggtgcccatccactgggcgacattccgcctcgccccgcatccgtggtccgagcccgccgaacgcctgctgaccgctgccgacgccgagcgggtacgcctgaccgtgccgattcccggtcagcgggtggacccggagtcgacgttcgacccgtggtggcggttctgaacc

SEQ ID NO: 194 - protein, 370 aa, Mycobacterium vaccae
MVRAALRYGFGTASLLAGGFVLRALQGTPAALGATPGEVAPVARRSPNYRDGKFVNLEPPSGITMDRDLQRMLLRDLANAASQGKPPGPIPLAEPPKGDPTPAPAAASWYGHSSVLIEVDGYRVLADPVWSNRCSPSRAVGPQRMHDVPVPLEALPAVDAVVISHDHYDHLDIDTIVALAHTQRAPFVVPLGIGAHLRKWGVPEARIVELDWHEAHRIDDLTLVCTPARHFSGRLFSRDSTLWASWVVTGSSHKAFFGGDTGYTKSFAEIGDEYGPFDLTLLPIGAYHPAFADIHMNPEEAVRAHLDLTEVDNSLMVPIHWATFRLAPHPWSEPAERLLTAADAERVRLTVPIPGQRVDPESTFDPWWRF

SEQ ID NO: 195 - DNA, 650 bp, Mycobacterium vaccae
gacacaccagcaccactgttaacctcgctagatcagtcggccgaacggaaggacagccgtgaccctgaaaaccctagtcaccagcatgaccgctggggcagcagcagccgcaacactcggcgctgccgccgtgggtgtgacctcgattgccgtcggtgcgggtgtcgccggcgcgtcgcccgcggtgctgaacgcaccgctgctttccgcccctgcccccgatctgcagggaccgctggtctccaccttgagcgcgctgtcgggcccgggctccttcgccggcgccaaggccacctacgtccagggcggtctcggccgcatcgaggcccgggtggccgacagcggatacagcaacgccgcggccaagggctacttcccgctgagcttcaccgtcgccggcatcgaccagaacggtccgatcgtgaccgccaacgtcaccgcggcggccccgacgggcgccgtggccacccagccgctgacgttcatcgccgggccgagcccgaccggatggcagctgtccaagcagtccgcactggccctgatgtccgcggtgggtgatctcccgcacgattctggtccgcagcgccgtcacatgtgtggcggcgctcgggctgggtgggtgcctgggcggctgcgcgcaagatgaacat

SEQ ID NO: 196 - protein, 159 aa, Mycobacterium vaccae
MTAGAAAAATLGAAAVGVTSIAVGAGVAGASPAVLNAPLLSAPAPDLQGPLVSTLSALSGPGSFAGAKATYVQGGLGRIEARVADSGYSNAAAKGYFPLSFTVAGIDQNGPIVTANVTAAAPTGAVATQPLTFIAGPSPTGWQLSKQSALALMSAVIAA

SEQ ID NO: 197 - protein, 285 aa, Mycobacterium vaccae
MQVRRVLGSVGAAVAVSAALWQTGVSIPTASADPCPDIEVIFARGTGAEPGLGWVGDAFVNALRPKVGEQSVGTYAVNYPAGFDFDKSAPMGAADASGRVQWMADNCPDTKLVLGGMSQGAGVIDLITVDPRPLGRFTPTPMPPRVADHVAAVVVFGNPLRDIRGGGPLPQMSGTYGPKSIDLCALDDPFCSPGFNLPAHFAYADNGMVEEAANFARLEPGQSVELPEAPYLHLFVPRGEVTLEDAGPLREGDAVRFTASGGQRVTATAPAEILVWEMHAGLGAA

SEQ ID NO: 198 - DNA, 743 bp, Mycobacterium vaccae
ggatccgcggcaccggctggtgacgaccaagtacaacccggcccgcacctggacggccgagaactccgtcggcatcggcggcgcgtacctgtgcatctacgggatggagggccccggcggctatcagttcgtcggccgcaccacccaggtgtggagtcgttaccgccacacggcgccgttcgaacccggaagtccctggctgctgcggtttttcgaccgaatttcgtggtatccggtgtcggccgaggagctgctggaattgcgagccgacatggccgcaggccggggctcggtcgacatcaccgacggcgtgttctccctcgccgagcacgaacggttcctggccgacaacgccgacgacatcgccgcgttccgttcccggcaggcggccgcgttctccgccgagcggaccgcgtgggcggccgccggcgagttcgaccgcgccgagaaagccgcgtcgaaggccaccgacgccgataccggggacctggtgctctacgacggtgacgagcgggtcgacgctccgttcgcgtcgagcgtgtggaaggtcgacgtcgccgtcggtgaccgggtggtggccggacagccgttgctggcgctggaggcgatgaagatggagaccgtgctgcgcgccccggccgacggggtggtcacccagatcctggtctccgctgggcatctcgtcgatcccggcaccccactggtcgtggtcggcaccggagtgcgcgcatgagcgccgtcga

SEQ ID NO: 199 - protein, 243 aa, Mycobacterium vaccae
DPRHRLVTTKYNPARTWTAENSVGIGGAYLCIYGMEGPGGYQFVGRTTQVWSRYRHTAPFEPGSPWLLRFFDRISWYPVSAEELLELRADMAAGRGSVDITDGVFSLAEHERFLADNADDIAAFRSRQAAAFSAERTAWAAAGEFDRAEKAASKATDADTGDLVLYDGDERVDAPFASSVWKVDVAVGDRVVAGQPLLALEAMKMETVLRAPADGVVTQILVSAGHLVDPGTPLVVVGTGVRA

SEQ ID NO: 200 - DNA, 858 bp, Mycobacterium vaccae
gaaatcccgcgtctgaaaccctcttttcgcggcgcccctcaggacggtaagggggccaagcggattgaaaaatgttcgctgaatgagcctgaaattgcgcgtggctcttggaaatcagcagcgatgggtttaccgtgtccactagtcggtccaaagaggaccactggttttcggaggttttgcatgaacaaagcagagctcatcgacgtactcactgagaagctgggctcggatcgtcggcaagcgactgcggcggtggagaacgttgtcgacaccatcgtgcgcgccgtgcacaagggtgagagcgtcaccatcacgggcttcggtgttttcgagcagcgtcgtcgcgcagcacgcgtggcacgcaatccgcgcaccggcgagaccgtgaaggtcaagcccacctcagtcccggcattccgtcccggcgctcagttcaaggctgttgtctctggcgcacagaagcttccggccgagggtccggcggtcaagcgcggtgtgaccgcgacgagcaccgcccgcaaggcagccaagaaggctccggccaagaaggctgccgcgaagaaggccgcgccggccaagaaggctccggcgaagaaggctgcgaccaaggctgcaccggccaagaaggccactgccgccaagaaggccgcgccggccaagaaggccactgccgccaagaaggctgcaccggccaagaaggctccggccaagaaggctgcgaccaaggctgcaccggccaagaaggctccggccaagaaggccgcgaccaaggctgcaccggccaagaaggctccggccgccaagaaggcgcccgccaagaaggctccggccaagcgcggcggacgcaagtaagtc

SEQ ID NO: 201 - protein, 223 aa, Mycobacterium vaccae
MNKAELIDVLTEKLGSDRRQATAAVENVVDTIVRAVHKGESVTITGFGVFEQRRRAARVARNPRTGETVKVKPTSVPAFRPGAQFKAVVSGAQKLPAEGPAVKRGVTATSTARKAAKKAPAKKAAAKKAAPAKKAPAKKAATKAAPAKKATAAKKAAPAKKATAAKKAAPAKKAPAKKAATKAAPAKKAPAKKAATKAAPAKKAPAAKKAPAKKAPAKRGGRK

SEQ ID NO: 202 - DNA, 570 bp, Mycobacterium vaccae
agacagacagtgatcgacgaaaccctcttccatgccgaggagaagatggagaaggccgtctcggtggcacccgacgacctggcgtcgattcgtaccggccgcgcgaaccccggcatgttcaaccggatcaacatcgactactacggcgcctccaccccgatcacgcagctgtccagcatcaacgtgcccgaggcgcgcatggtggtgatcaagccctacgaggcgagccagctgcgcctcatcgaggatgcgatccgcaactccgacctcggcgtcaatccgaccaacgacggcaacatcatccgggtgtcgatcccgcagctcaccgaggagcgccgccgcgacctggtcaagcaggccaaggccaagggcgaggacgccaaggtgtcggtgcgcaacatccgtcgcaaggcgatggaggaactctcccggatcaagaaggacggcgacgccggcgaagaccaagtgacccgcgccgagaaggatctcgacaagagcacccaccagtacacgaatcagatcgacgaactggtcaagcacaaggaaggcgagttgctggaggtctgacca

SEQ ID NO: 203 - protein, 187 aa, Mycobacterium vaccae
  UNSURE (186)...(186)
VIDETLFHAEEKMEKAVSVAPDDLASIRTGRANPGMFNRINIDYYGASTPITQLSSINVPEARMVVIKPYEASQLRLIEDAIRNSDLGVNPTNDGNIIRVSIPQLTEERRRDLVKQAKAKGEDAKVSVRNIRRKAMEELSRIKKDGDAGEDEVTRAEKDLDKSTHQYTNQIDELVKHKEGELLEVXP

SEQ ID NO: 204 - DNA, 1364 bp, Mycobacterium vaccae
cgacctccacccgggcgtgaggccaaccactaggctggtcaccagtagtcgacggcacacttcaccgaaaaaatgaggacagaggagacacccgtgacgatccgtgttggtgtgaacggcttcggccgtatcggacgcaacttcttccgcgcgctggacgcgcagaaggccgaaggcaagaacaaggacatcgagatcgtcgcggtcaacgacctcaccgacaacgccacgctggcgcacctgctgaagttcgactcgatcctgggccggctgccctacgacgtgagcctcgaaggcgaggacaccatcgtcgtcggcagcaccaagatcaaggcgctcgaggtcaaggaaggcccggcggcgctgccctggggcgacctgggcgtcgacgtcgtcgtcgagtccaccggcatcttcaccaagcgcgacaaggcccagggccacctcgacgcgggcgccaagaaggtcatcatctccgcgccggccaccgatgaggacatcaccatcgtgctcggcgtcaacgacgacaagtacgacggcagccagaacatcatctccaacgcgtcgtgcaccacgaactgcctcggcccgctggcgaaggtcatcaacgacgagttcggcatcgtcaagggcctgatgaccaccatccacgcctacacccaggtccagaacctgcaggacggcccgcacaaggatctgcgccgggcccgcgccgccgcgctgaacatcgtgccgacctccaccggtgccgccaaggccatcggactggtgctgcccgagctgaagggcaagctcgacggctacgcgctgcgggtgccgatccccaccggctcggtcaccgacctgaccgccgagctgggcaagtcggccaccgtggacgagatcaacgccgcgatgaaggctgcggccgagggcccgctcaagggcatcctcaagtactacgacgccccgatcgtgtccagcgacatcgtcaccgatccgcacagctcgatcttcgactcgggtctgaccaaggtcatcgacaaccaggccaaggtcgtgtcctggtacgacaacgagtggggctactccaaccgcctcgtcgacctggtcgccctggtcggcaagtcgctgtaggggcgagcgaagcgacgggagaacagaggcgccatggcgatcaagtcactcgacgaccttctgtccgaaggggtgacggggcggggcgtactcgtgcgctccgacctgaacgtccccctcgacggcgacacgatcaccgacccggggcgcatcatcgcctcggtgccgacgttgaaggcgttgagtgacgccggcgccaaggtggtcgtcaccgcgcatctgggcaggcccaagggtgagccggatcc

SEQ ID NO: 205 - protein, 340 aa, Mycobacterium vaccae
VTIRVGVNGFGRIGRNFFRALDAQKAEGKNKDIEIVAVNDLTDNATLAHLLKFDSILGRLPYDVSLEGEDTIVVGSTKIKALEVKEGPAALPWGDLGVDVVVESTGIFTKRDKAQGHLDAGAKKVIISAPATDEDITIVLGVNDDKYDGSQNIISNASCTTNCLGPLAKVINDEFGIVKGLMTTIHAYTQVQNLQDGPHKDLRRARAAALNIVPTSTGAAKAIGLVLPELKGKLDGYALRVPIPTGSVTDLTAELGKSATVDEINAAMKAAAEGPLKGILKYYDAPIVSSDIVTDPHSSIFDSGLTKVIDNQAKVVSWYDNEWGYSNRLVDLVALVGKSL

SEQ ID NO: 206 - DNA, 522 bp, Mycobacterium vaccae
acctacgagttcgagaacaaggtcacgggcggccgcatcccgcgcgagtacatcccgtcggtggatgccggcgcgcaggacgccatgcagtacggcgtgctggccggctacccgctggttaacgtcaagctgacgctgctcgacggtgcctaccacgaagtcgactcgtcggaaatggcattcaaggttgccggctcccaggtcatgaagaaggctgccgcccaggcgcagccggtgatcctggagccagtgatggcggtcgaggtcacgacgcccgaggattacatgggtgaagtgagcggcgacctgaactcccgccgtggtcagatccaggccatggaggagcggagcggtgctcgtgtcgtgaaggcgcaggttccgctgtcggagatgttcggctacgtcggagaccttcggtcgaagacccagggccgggccaactactccatggtgttcgactcgtacgccgaagttccggcgaacgtgtcgaaggagatcatcgcgaaggcgacgggccagtaa

SEQ ID NO: 207 - protein, 173 aa, Mycobacterium vaccae
TYEFENKVTGGRIPREYIPSVDAGAQDAMQYGVLAGYPLVNVKLTLLDGAYHEVDSSEMAFKVAGSQVMKKAAAQAQPVILEPVMAVEVTTPEDYMGEVIGDLNSRRGQIQAMEERSGARVVKAQVPLSEMFGYVGDLRSKTQGRANYSMVFDSYAEVPANVSKEIIAKATGQ

SEQ ID NO: 208 - protein, 12 aa, Mycobacterium vaccae
ALPQLTDEQRAA